(19) United States
(12) Patent Application Publication
(10) Pub. No.: US 2023/0040275 A1
(43) Pub. Date: Feb. 9, 2023

(54) SECRETABLE PROTEIN INDUCED IMMUNE TOLERIZATION AND TREATMENT OF AUTOIMMUNE, ALLERGIC AND OTHER DISEASES AND DISORDERS

(71) Applicant: SPARK THERAPEUTICS, INC., Philadelphia, PA (US)

(72) Inventors: David William ANDERSON, Newtown Square, PA (US); Brittney L. GURDA, Collegeville, PA (US); Federico MINGOZZI, Philadelphia, PA (US); Mustafa N. YAZICIOGLU, Broomall, PA (US); William QUINN, Philadelphia, PA (US)

(73) Assignee: SPARK THERAPEUTICS, INC., Philadelphia, PA (US)

(21) Appl. No.: 17/777,954

(22) PCT Filed: Nov. 19, 2020
(86) PCT No.: PCT/US2020/061356

§ 371 Date: May 18, 2022

Related U.S. Application Data
(60) Provisional application No. 62/937,581, filed on Nov. 19, 2019.

(51) Int. Cl. C12N 15/62; A61K 39/35; A61K 39/00; C07K 14/705; C12N 15/86; A61K 48/00; A61P 37/06; A61K 31/436; C07K 14/47; C07K 14/005

(52) U.S. Cl. CPC C12N 15/62; A61K 39/35; A61K 39/0008; C07K 14/70503; C12N 15/86; A61K 48/0058; A61P 37/06; A61K 31/436; C07K 14/47; C07K 14/005; C07K 2319/03; C12N 2750/14143; C12N 2750/14171; A61K 2039/53; C12N 2750/14134; A61K 2039/55555; A61K 2039/577; C12N 2750/14151; C07K 2319/02; C12N 2770/20034

(57) ABSTRACT

Nucleic acids encoding fusion proteins that contain an unwanted antigen and a leader sequence for cell secretion are described. Also described are expression cassettes, vectors, cells, and cell lines containing the nucleic acids, as well as methods of using the nucleic acids to treat autoimmune, allergic and other diseases and disorders, such as multiple sclerosis.

### CROSS REFERENCE TO RELATED APPLICATION

This application claims priority to U.S. Provisional Application No. 62/937,581 filed on Nov. 19, 2019, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 17, 2020, is named “Sequence Listing SPK168PCT.txt” and is 215 kilobytes in size.

### INTRODUCTION

Mature myelin oligodendrocyte glycoprotein (MOG) is associated with the bi-lipid layer. MOG is characterized by an IgV-like extracellular domain, a single-bypass transmembrane protein, a membrane-associated domain, and a cytoplasmic tail. The extracellular IgV-like domain is denoted herein as mini-MOG (mMOG). MOG is predominantly found in membranes of oligodendrocytes and contributes a small amount to the final composition of myelin. Transcript analysis identifies this region as exon 2. The length of this protein sequence varies among species.

AAV gene therapy has been used with hepatic-restricted transgene expression to induce immune tolerance to therapeutic proteins, inter alia, FIX (see Mingozzi et al., 2003, J. Clin. Invest., 111:1347-1356), lysosomal storage enzymes ASM and GAA (see LoDuca et al., 2009, Curr. Gene Ther., 9:104-114; see Table 1 and text), and full-length, non-secreted MOG (see Keeler et al., 2017, Mol. Ther., 26:173-183).

There is a need for improved constructs for expressing unwanted antigens for treatment of autoimmune, allergic and other diseases and disorders, such as multiple sclerosis.

### SUMMARY

The invention provides nucleic acids encoding fusion proteins. In one embodiment, a fusion protein comprises an unwanted antigen and a leader sequence for cell secretion.

The invention also provides expression cassettes comprising nucleic acids encoding fusion proteins. In one embodiment, an expression cassette comprises an expression control element operably linked to a nucleic acid encoding a fusion protein comprising an unwanted antigen and a leader sequence for cell secretion.

In various embodiments, the unwanted antigen comprises a self-antigen, autoantigen or protein or peptide that has structural similarity or sequence identity to the self-antigen or the autoantigen.

In various embodiments, the protein or peptide that has structural similarity or sequence identity to the self-antigen or the autoantigen is a microbial protein or peptide.

In various embodiments, the unwanted antigen comprises an allergen. In particular aspects, the allergen comprises a plant, insect, or animal allergen.

In various embodiments, the unwanted antigen is not a protein or peptide for correcting or replacing a defective or unexpressed gene or protein in a subject.

In various embodiments, the nucleic acid does not comprise a gene for replacing a defective or unexpressed gene or protein in a subject.

In various embodiments, the unwanted antigen binds to or activates T regulatory cells (Tregs) when expressed in a subject. In particular aspects, the Tregs are Fox P3+/CD4+/CD25+Tregs.

In various embodiments, the unwanted antigen causes deletion or exhaustion of effector T cells when expressed in a subject.

In various embodiments, the unwanted antigen is truncated or is a subsequence of a full length native/wildtype unwanted antigen.

In various embodiments, the unwanted antigen is an immune tolerizing unwanted antigen, wherein the immune tolerizing unwanted antigen suppresses, reduces or inhibits a cell-mediated or antibody mediated immune response when expressed in a subject.

In various embodiments, the leader sequence comprises or consists of an amino acid sequence of any of SEQ ID NOs:13-25.

In various embodiments, the leader sequence is encoded by a polynucleotide sequence of any of SEQ ID NOs:26-38.

In various embodiments, the unwanted antigen comprises a mammalian protein or peptide.

In various embodiments, the unwanted antigen comprises a human protein or peptide.

In various embodiments, the unwanted antigen comprises an antigen having or consisting of the amino acid sequence of any of SEQ ID NOs: 5-8, 51-460, 463-469, 477-484, or a subsequence of any of SEQ ID NOs: 5-8, 51-460, 463-469, 477-484 capable of inducing an immune response in a subject when expressed in the subject.

In various embodiments, the unwanted antigen comprises a myelin oligodendrocyte glycoprotein (MOG), myelin basic protein (MBP), proteolipid protein (PLP), or subsequence thereof.

In various embodiments, the MOG lacks all or a part of a transmembrane domain.

In various embodiments, the MOG comprises or consists of amino acids 1-117 of a mature MOG.

In various embodiments, the MOG subsequence is a subsequence of an extracellular domain of mature MOG or a subsequence of a transmembrane domain of a mature MOG.

In various embodiments, the MOG comprises or consists of amino acids 35-55, 118-132, 181-195, or 186-200 of a mature MOG.

In various embodiments, the MOG comprises or consists of amino acids 1-20, 11-30, 21-40, 31-50, etc. of a mature MOG.

In various embodiments, the MOG comprises or consists of the amino acid sequence of any of SEQ ID NOs:5-8, 246-251, 442-460, and 463-469, or a subsequence thereof capable of inducing an immune response in a subject when expressed in the subject.

In various embodiments, the MBP includes a transmembrane domain of a mature MBP.

In various embodiments, the MBP lacks all or a part of a transmembrane domain of a mature MBP. In various embodiments, the MBP subsequence is a subsequence of an extracellular domain or a subsequence of a transmembrane domain of a mature MBP.

In various embodiments, the PLP lacks all or a part of a transmembrane domain of a mature PLP.

In various embodiments, the PLP subsequence is a subsequence of an extracellular domain or a subsequence of a transmembrane domain of a mature PLP.

In various embodiments, the PLP comprises or consists of amino acids 37-63, 89-151, 178-233, or 261-277 of a mature PLP.

In various embodiments, the expression cassette further comprises one or more additional polynucleotide elements positioned 5′ and/or 3′ of the nucleic acid or expression control element.

In various embodiments, the expression control element is positioned 5′ of the nucleic acid.

In various embodiments, the expression control element comprises an ApoE/hAAT enhancer/promoter sequence, a CAG promoter, cytomegalovirus (CMV) immediate early promoter/enhancer, Rous sarcoma virus (RSV) promoter/enhancer, SV40 promoter, dihydrofolate reductase (DHFR) promoter, or chicken β-actin (CBA) promoter.

In various embodiments, the expression cassette further comprises a poly-adenylation sequence positioned 3′ of the nucleic acid.

In various embodiments, the expression cassette further comprises an intron, the intron optionally positioned between the expression control element and the nucleic acid or optionally positioned within the nucleic acid.

In various embodiments, the expression cassette is positioned between one or more 5′ and/or 3′adeno-associated virus (AAV) inverted terminal repeat(s) (ITR(s)).

In various embodiments, the one or more 5′ and/or 3′ ITR(s) comprise AAV serotype AAV1, AAV2, AAV3, AAV4, AAV5, AAV6, AAV7, AAV8, AAV9, AAV10, AAV11, AAV12, AAV-2i8, AAV3B, Rh74 or Rh10 ITR.

In various embodiments, the AAV ITR(s) comprises a mutated, modified or variant AAV ITR that is not processed by AAV Rep protein.

In various embodiments, the AAV ITR(s) comprises a mutated, modified or variant AAV ITR that allows or facilitates formation of a self-complementary expression cassette.

In various embodiments, the mutated, modified or variant AAV ITR has a deleted D sequence, and/or a mutated, modified or variant terminal resolution site (TRS) sequence.

In various embodiments, the nucleic acid, expression control element, poly-adenylation sequence or ITR has reduced CpG dinucleotides.

In various embodiments, the nucleic acid, expression control element, poly-adenylation sequence or ITR has increased CpG dinucleotides.

In various embodiments, the expression cassette is comprised in a viral particle.

In various embodiments, the expression cassette is comprised in a lenti-viral particle.

In various embodiments, the expression cassette is comprised in a lipid nanoparticle (LNP) composition.

In various embodiments, the nucleic acid encoding the fusion protein is an mRNA and is comprised in an LNP composition.

In various embodiments, the expression cassette is comprised in a recombinant adeno associated virus (rAAV) particle.

In various embodiments, the expression cassette comprises in 5′→3′ orientation a first AAV ITR; a promoter operable in mammalian cells; the nucleic acid; a polyadenylation signal; and optionally a second AAV ITR.

In various embodiments, the rAAV particle comprises a VP1, VP2 or VP3 sequence 60% or more identical to a VP1, VP2 or VP3 sequence of AAV serotype AAV1, AAV2, AAV3, AAV4, AAV5, AAV6, AAV7, AAV8, AAV9, AAV10, AAV11, AAV12, AAV-2i8, AAV3B, Rh74, Rh10, SPK1 (SEQ ID NO:1), SPK2 (SEQ ID NO:2) VP1, VP2 and/or VP3, or a hybrid or chimera of any of the foregoing AAV serotypes.

In various embodiments, the rAAV particle comprises VP1, VP2 and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV10, AAV11, AAV12, AAV-2i8, AAV3B, Rh10, Rh74, SPK1 (SEQ ID NO:1) and SPK2 (SEQ ID NO:2) VP1, VP2 and/or VP3 capsid proteins.

In various embodiments, a pharmaceutical composition comprises one or more nucleic acids, expression vectors, viral particles, lenti-viral particles, and/or rAAV particles in a biologically compatible carrier or excipient.

In various embodiments, a pharmaceutical composition comprises one or more viral particles in a biologically compatible carrier or excipient.

In various embodiments, a pharmaceutical composition comprises one or more lenti-viral particles in a biologically compatible carrier or excipient.

In various embodiments, a pharmaceutical composition comprises one or more rAAV particles in a biologically compatible carrier or excipient.

In various embodiments, a pharmaceutical composition comprises empty AAV capsids.

In various embodiments, the ratio of the empty AAV capsids to the rAAV particle is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100.

In various embodiments, the ratio of the empty AAV capsids to the rAAV particle is about 2:1, 3:1, 4:1, 5:1, 6:1, 7:1, 8:1, 9:1, or 10:1.

In various embodiments, the pharmaceutical composition further comprises a surfactant.

The invention further provides methods of suppressing, reducing or inhibiting a cell-mediated or antibody mediated immune response to an unwanted antigen in a mammal. In one embodiment, a method includes providing an expression cassette, particle or pharmaceutical composition or LNP composition as set forth herein; and administering an amount of the expression cassette, particle, pharmaceutical composition or LNP composition to the mammal, wherein the fusion protein is expressed in the mammal sufficient to suppress, reduce or inhibit a cell-mediated or antibody mediated immune response to the unwanted antigen.

The invention additionally provides methods of inducing tolerance in a mammal to an unwanted antigen. In one embodiment, a method includes providing an expression cassette, particle, or pharmaceutical composition or LNP composition as set forth herein; and administering an amount of the expression cassette, particle, pharmaceutical or LNP composition to the mammal, wherein the fusion protein is expressed in the mammal sufficient to induce tolerance to the unwanted antigen.

The invention moreover provides methods of treating a human in need of a fusion protein. In one embodiment, a method includes providing an expression cassette, particle, or pharmaceutical composition or LNP composition as set forth herein; and administering an amount of the expression cassette, particle, pharmaceutical or LNP composition to the human, wherein the fusion protein is expressed in the human.

In various embodiments, the human has an autoimmune disease or disorder.

In various embodiments, the human has an allergy or allergic disease or disorder.

In various embodiments, the human has a disease or disorder set forth in any of Tables 1 and 2.

In various embodiments, the human has multiple sclerosis, anti-MAG peripheral neuropathy, type 1 diabetes, Graves disease, rheumatoid arthritis, proteoglycan induced arthritis (PGIA) or myasthenia gravis.

In various embodiments, administering is intravenous, intra-arterial, intra-cavity, intra-mucosal, or via catheter.

In various embodiments, the rAAV particle is administered in a range from about 1×10⁸ to about 1×10¹⁴ AAV vector genomes per kilogram (vg/kg) of the weight of the human.

In various embodiments, a method further includes administering an immunosuppressive agent.

In various embodiments, a method further includes administering an anti-inflammatory agent.

In various embodiments, a method further includes administering a steroid.

In various embodiments, the immunosuppressive agent comprises rapamycin, a cyclosporine (e.g., cyclosporine A), mycophenolate, rituximab or a derivative thereof.

In various embodiments, the method reduces, decreases or inhibits one or more symptoms of the auto immune disease or disorder or allergy or allergic disorder.

The invention additionally provides cells comprising the nucleic acids and expression cassettes set forth herein.

In various embodiments, a cell produces the viral particle, lentiviral particle or rAAV particle.

The invention still further provides methods of producing a plurality of rAAV particles.

In one embodiment, a method includes introducing an AAV vector genome comprising an expression cassette as set forth herein into a packaging helper cell; and culturing the helper cell under conditions to produce the rAAV particles.

In another embodiment, a method includes introducing and expression cassette as set forth herein into a packaging helper cell; and culturing the helper cells under conditions to produce the rAAV particles.

In various embodiments of the methods of producing rAAV particles, a method further includes isolating or purifying the rAAV particles.

In various embodiments of the cells and methods of producing rAAV particles, the cell comprises mammalian cells.

In various embodiments of the cells and methods of producing rAAV particles, the cell provides helper functions that package the AAV vector genome into the rAAV particle.

In various embodiments of the cells and methods of producing rAAV particles, the cell provides AAV helper functions.

In various embodiments of the cells and methods of producing rAAV particles, the cell provides AAV Rep and/or Cap proteins.

In various embodiments of the cells and methods of producing rAAV particles, the cell is stably or transiently transfected with polynucleotide(s) encoding AAV Rep and/or Cap protein sequence(s).

In various embodiments of the cells and methods of producing rAAV particles, the cell comprises HEK-293 cells.

### DESCRIPTION OF DRAWINGS

FIG. 1 shows data indicating that mature MOG is retained in the plasma membrane of cells. Lanes A, C-E & M: Mature MOG is enriched in the plasma membrane. No signal is seen in GFP transfected control cells (lane B & L). Lanes F-J & K, M-P: Faint bands are evident in cell lysate equivalent and media.

FIG. 2 shows a qualitative analysis of mini-MOG secretion into cell culture media when fused to various leader sequences. GAPDH is used as a loading control. GAPDH is not visualized in the cell media indicating that cell debris/cell lysate is not present in the cell culture media and thus, this material does not contribute to the presence of secreted mMOG in the media. WT leader: wildtype, or native, mini-MOG, human chymotrypsinogen B2 signal peptide (“Sp7”), HC7 leader, and Gaussia leader. HC7s is a control that included the native MOG leader signal in addition to the HC7 signal peptide; the duplication of signal sequences led to a lack of MOG protein expression.

FIG. 3 shows that removal of leader sequence sequesters mini-MOG within the cell and no mMOG is secreted. Note the absence of mMOG in the media in the 48 and 72 hours (hr) post-transfection samples, while mMOG is identified in the cell lysate taken at 72 hr post-transfection.

FIG. 4 shows glycosylation profile of mMOG. Equivalent volumes of mMOG lysates and cell media (72 hr timepoint) were treated with Endo-Hf or PNGaseF. Controls were treated the same, but no enzyme was added. Cell lysates indicate two populations—one with high mannose (or susceptible to Endo-Hf) and one susceptible to PNGaseF, indicating complex glycan cleavage. A single population of mMOG is evident in the media as PNGaseF susceptible. Secreted mMOG is thus glycosylated with a complex glycan. These findings are similar across all leader sequences evaluated indicating that the leader does not alter base protein attributes.

FIG. 5 shows that mMOG packaged into AAV (Spk2.mMOG) transduced Huh7 cells and was expressed in the Huh7 cells. Crude lysate vectors were generated in HEK293 cells using the Spk2 capsid. Huh7 cells were then incubated with the crude lysates for 24 hr and the media was replaced with fresh culture media. Huh7 cells and media were harvested 72 hr post-infection and assayed for mMOG expression.

FIG. 6 shows a schematic of a prevention strategy in C57B/6 mice described in Example 6. 8-9 week old male or female C57B/6 mice were IV injected via tail vein with AAV.ApoE/hAAT.MOG vectors 2 weeks prior to MOG35-55-EAE induction. After EAE induction clinical disease was followed. All groups were sacrificed when untreated control groups reached a mean clinical score (MCS) of 3.0-3.5.

FIG. 7 shows graphs of individual MCS scores in prevention studies of AAV-treated EAE mice. Four groups of mice (N=6) were injected with 1E11vgs of AAV vectors 2-weeks (day −14) prior to EAE induction (day 0) and clinical disease was followed until the control group reached endpoint (day14-18) at MCS=3.0-3.5; specifically, FIG. 7A shows that full-length MOG (AAV.fMOG) treated mice never developed clinical disease (MCS=0), FIG. 7B shows that a soluble portion of fMOG, termed mMOG, prevented disease in 4/6 mice, FIG. 7C shows that a secreted version of mMOG, Sp7.mMOG, protected 5/6 mice from clinical EAE, and FIG. 7D shows that control vectors were irrelevant to the disease and mice in this group succumbed to clinical EAE; *Mice that reached clinical disease endpoint; error bars represent the SEM of average clinical scores from 5 blinded scientists scoring the animals; each data point with a line represents an individual animal.

FIG. 8 shows images of histological analysis of lumbar spinal cord from AAV-treated EAE mice. Each image is a representative animal from each group. Clinical scores, or MCS, for each image are identified as EAE #. Images were generated from H&E sections, left image vs corresponding luxol fast blue section, right image; specifically, FIG. 8A shows that no lesions were identified in AAV.fMOG treated mice, N=6, FIG. 8B shows that lesions were found in only 1 out of 4 AAV.mMOG analyzed mice which corresponded with clinical EAE, FIG. 8C shows that small lesions were identified in 3 out of 6 analyzed AAV.Sp7.mMOG spinal cord sections, and FIG. 8D shows that all analyzed control spinal cord sections had multiple infiltrating lesions.

FIG. 9 shows MOG protein analysis in liver lysates of AAV-treated livers on WES 12-230 kDa, with the following lane assignments: (A) 50 ngs total protein (tp) AAV.fMOG runs at ˜28 kDa, (B) 1 ug tp AAV.mMOG, (C) 1 ug tp AAV.Sp7.mMOG, and the mMOG fragments of lanes B and C runs at ˜18 kDa, and (D) 1 ug tp AAV control serves as negative control for MOG protein. Lysates were probed with goat anti-mouse MOG and HRP conjugated anti-goat.

FIG. 10 shows a schematic of a study described in Example 7; EAE was induced in 8-10 week-old juvenile C57/B6 mice and clinical scores were monitored. When animals reached a MCS ˜2.5-3.0 they were randomly assigned to a treatment group and dosed with the assigned rescue treatment. AAV vectors were dosed IV tail vein at 1E11vgs total and rapamycin was given 5 mg/kg IP at 0, 48, and 96 hours post-rescue. Groups were followed for an additional two weeks and sacrificed.

FIG. 11 shows a plot of the MCS of AAV rescue groups. EAE mice were randomly assigned to rescue groups when they reached an MCS ˜2.5-3.0 (approx. AAV rescue). Groups were scored for clinical disease for an additional two weeks following rescue. Rescue groups were as follows were AAV was dosed at 1E11vgs via tail vein and rapamycin was given IP at 5 mg/kg on 0, 48, and 96 hours post-rescue: AAV.fMOG (closed square), AAV.fMOG+rapa (open square), AAV.Sp7.mMOG (closed circle), AAV.Sp7.mMOG+rapa (open circle), AAV control+rapa (closed down-triangle), rapamycin only (left-closed triangle), and EAE only (closed diamond). *Two EAE mice were euthanized early due to extensive clinical signs. The rise in MCS reflects the loss of these animals on study.

### DETAILED DESCRIPTION

The disclosed methods can be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The invention provides, inter alia, compositions and methods for inducing, providing, enhancing and/or stimulating immune tolerance in a subject. The invention also provides compositions and methods for suppressing, inhibiting, reducing and/or decreasing an immune response in a subject.

As used herein, an “immune tolerizing unwanted antigen” refers to an unwanted antigen that suppresses, reduces or inhibits a cell-mediated or antibody mediated immune response.

Immune responses to which the invention is directed include humoral and cell mediated immune responses. In the context of an autoimmune disease or disorder, preventing, suppressing, inhibiting, reducing, decreasing or otherwise downregulating such immune responses can lead to treatment of autoimmunity and/or inflammation. In the context of an allergic disease or disorder or an allergic reaction, preventing, suppressing, inhibiting, reducing, decreasing or otherwise down regulating such immune responses can lead to treatment of the allergic disease or disorder or allergic reaction. Accordingly, the invention further provides methods of treating autoimmune diseases and disorders, and methods of treating allergic diseases and disorders including allergic reactions.

Compositions of the invention include nucleic acids that encode a fusion protein, wherein the fusion protein comprises an unwanted antigen and a leader sequence for cell secretion. Such nucleic acids can be incorporated into an expression cassette in which an expression control element is operably linked to the nucleic acid encoding the fusion protein. Such compositions are useful in all methods of the invention disclosed herein.

As used herein, an “unwanted antigen” is a self-antigen or autoantigen that is able to induce, provide, enhance and/or stimulate immune tolerance against the antigen itself or a protein that includes all or a portion of the antigen and/or that suppresses, inhibits, reduces and/or decreases an immune response directed towards the antigen itself or a protein that includes all or a portion of the antigen. An unwanted antigen as used herein also includes allergens or allergenic antigens that can induce, provide, enhance and/or stimulate immune tolerance against the allergen as well as allergens and allergenic antigens that suppress, inhibit, reduce and/or decrease an immune response directed towards the allergen or an entity that includes the allergen.

Unwanted antigens as set forth herein also include allogenic antigens or transplantation antigens or minor histocompatibility antigens that can lead to rejection of a cell, tissue or organ after their transplantation into a subject. The subject typically recognizes the transplanted cell, tissue or organ as foreign and develops an immune response against the cell, tissue or organ. Accordingly, the invention methods are directed to preventing or reducing rejection of a cell, tissue or organ after transplant into a subject.

In certain embodiments, unwanted antigens include proteins or peptides that that contain one or more changes in an amino acid variant sequence compared to that of the wild-type. The term “variant sequence” includes, e.g., amino acid insertions, additions, substitutions and deletions.

In various embodiments, the unwanted antigen is not a protein or peptide for correcting or replacing a defective or unexpressed gene or protein in a subject. As used herein, a “defective or unexpressed gene or protein” refers to a gene or protein for which a subject has a deficiency in a functional gene product, or produces an aberrant, partially functional or non-functional gene product, which can lead ro disease.

Although not wishing to be bound by any theory or particular mechanism, it is believed that the unwanted antigen functions by binding to or activating T regulatory cells (Tregs) thereby preventing, suppressing, inhibiting, reducing, decreasing or otherwise down regulating an immune response. This binding to or activation of Tregs in turn can lead to immune tolarization against the self-antigen or autoantigen. It is also possible that expression of the unwanted antigen causes exhaustion or deletion of effector T cells.

As used herein, a “leader” sequence is an amino acid sequence that when linked to a protein provides or facilitates enhanced secretion of the linked protein from the cell in which it is expressed. A leader sequence as used herein can also be referred to as a secretion sequence or a signal peptide. Such leader and secretion sequences or signal peptides are intended to provide or facilitate cell secretion but may not always facilitate secretion if they are linked to a protein that has a signal sequence that may prevent secretion of the protein.

Signal peptides are short peptides (typically 25 to 30 amino acids in length) located in the N-terminus of proteins, carrying information for protein secretion. Signal peptides direct proteins to or through the endoplasmic reticulum secretory pathway. By “enhanced” secretion, it is meant that the relative proportion of the polypeptide synthesized by the cell that is secreted from the cell is increased when it is fused to the leader sequence; it is not necessary that the absolute amount of secreted protein is also increased. In certain embodiments, essentially all (i.e., at least 95%, 97%, 98%, 99% or more) of the polypeptide is secreted. It is not necessary, however, that essentially all or even most of the polypeptide is secreted, as long as the level of secretion is enhanced as compared with the native polypeptide having its native or naturally occurring signal peptide. Generally, secretory signal sequences are cleaved within the endoplasmic reticulum and, in certain embodiments, the secretory signal sequence is cleaved prior to secretion. It is not necessary, however, that the secretory signal sequence is cleaved as long as secretion of the polypeptide from the cell is enhanced and the polypeptide is functional. Thus, in certain embodiments, the secretory signal sequence is partially or entirely retained. The secretory signal sequence can be derived in whole or in part from the secretory signal of a secreted polypeptide (i.e., from the precursor) and/or can be in whole or in part synthetic. The length of the secretory signal sequence is not critical; generally, known secretory signal sequences are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance secretion of an operably linked polypeptide. The secretory signal sequences of the instant invention can comprise, consist essentially of, or consist of a naturally occurring secretory signal sequence or a modification thereof. Numerous secreted proteins and sequences that direct secretion from the cell are known in the art, including those described in Owji et al., Eur. J. Cell Biol. 97:422-441 (2018). The secretory signal sequence of the instant invention can further be in whole or in part synthetic or artificial. Synthetic or artificial secretory signal peptides are known in the art, see, e.g., Barash et al., Biochem. Biophys. Res. Comm. 294:835-42 (2002).

Any suitable signal peptide known to those skilled in the art in view of the present disclosure can be used in the invention. Examples of signal peptides include, but are not limited to, those found from the Signal Peptide Database (website: www.signalpeptide.de/). Examples of signal peptides suitable for the present invention include, but are not limited to, wild-type C1 inhibitor signal peptide, a human chymotrypsinogen B2 signal peptide (18 amino acid signal peptide of NCBI reference sequence NP_001020371)), ALB signal peptide, ORM1 signal peptide, TF signal peptide, AMBP signal peptide, LAMP1 signal peptide, BTN2A2 signal peptide, CD300 signal peptide, NOTCH2 signal peptide, STRC signal peptide, AHSG signal peptide, SYN1 signal peptide, SYN2 signal peptide, SYN3 signal peptide, SYN4 signal peptide, secrecon (human signal sequence described in Barash et al., Biochem Biophys Res Commun. 2002; 294: 835-842), mouse IgKVIII, human IgKVIII, CD33, tPA, a-1 antitrypsin signal peptide, native secreted alkaline phosphatase (SEAP). Any conventional signal sequence that directs proteins through the endoplasmic reticulum secretory pathway, including variants of the above-mentioned signal peptides, can be used in the present invention.

In some embodiments, an unwanted antigen comprises an autoimmune disease protein or a subsequence thereof. An autoimmune disease protein includes any antigen (such as a protein, subsequence thereof, or a peptide) that contributes to initiation and/or progression of an autoimmune disease. Such autoimmune disease proteins can be derived from other organisms, such as microorganisms because the sequence or structure of the proteins from the other organisms mimic the self-antigen or autoantigen.

Exemplary autoimmune disease proteins include myelin oligodendrocyte glycoprotein (MOG, e.g., for multiple sclerosis), myelin basic protein (MBP, e.g., for multiple sclerosis), proteolipid protein (PLP, e.g., for multiple sclerosis), myelin-associated glycoprotein (MAG, e.g., for anti-MAG peripheral neuropathy), insulin (e.g., for type 1 diabetes), islet-specific glucose-6-phosphatase catalytic subunit-related protein (IGRP, e.g., for type 1 diabetes), preproinsulin (e.g., for type 1 diabetes), glutamic decarboxylase (GAD, e.g., for type 1 diabetes), tyrosine phosphatase like autoantigen (e.g., for type 1 diabetes), insulinoma antigen-2 (e.g., for type 1 diabetes), islet cell antigen (e.g., for type 1 diabetes); thyroid stimulating hormone (TSH) receptor (e.g., for Graves disease), thyrotropin receptor (e.g., for Graves disease), chondroitin sulfate proteoglycan 1 (e.g., for rheumatoid arthritis), CD4+ T cell epitope (e.g., GRVRVNSAY), e.g., for proteoglycan induced arthritis (PGIA) or rheumatoid arthritis), and acetylcholine receptor (AChR) or muscle specific kinase (MuSK) (e.g., for myasthenia gravis).

In some embodiments, an unwanted antigen is a mammalian myelin oligodendrocyte glycoprotein (MOG), myelin basis protein (MBP), proteolipid protein (PLP), or a subsequence thereof. In some embodiments, an unwanted antigen is a human protein, such as human myelin basis protein (MBP), a human proteolipid protein (PLP), a human myelin oligodendrocyte glycoprotein (MOG), or a subsequence thereof.

In certain embodiments, the numbering of the amino acids in a MOG is in reference to the numbering of the amino acids in SEQ ID NO:5. In certain embodiments, the numbering of the amino acids in a MOG is in reference to the numbering of the amino acids in SEQ ID NO:6. In certain embodiments, the numbering of the amino acids in a MOG is in reference to the numbering of the amino acids in SEQ ID NO:7. In certain embodiments, the numbering of the amino acids in a MOG is in reference to the numbering of the amino acids in SEQ ID NO:8.

In certain embodiments, the numbering of the amino acids in a mature MOG is in reference to the numbering of the amino acids in SEQ ID NO:466. In certain embodiments, the numbering of the amino acids in a MOG is in reference to the numbering of the amino acids in SEQ ID NO:467. In certain embodiments, the numbering of the amino acids in a MOG is in reference to the numbering of the amino acids in SEQ ID NO:468. In certain embodiments, the numbering of the amino acids in a MOG is in reference to the numbering of the amino acids in SEQ ID NO:469.

In certain embodiments, the numbering of the amino acids in a MBP is in reference to the numbering of the amino acids in SEQ ID NO:40. In certain embodiments, the numbering of the amino acids in a MBP is in reference to the numbering of the amino acids in SEQ ID NO:41. In certain embodiments, the numbering of the amino acids in a MBP is in reference to the numbering of the amino acids in SEQ ID NO:42. In certain embodiments, the numbering of the amino acids in a MBP is in reference to the numbering of the amino acids in SEQ ID NO:43. In certain embodiments, the numbering of the amino acids in a MBP is in reference to the numbering of the amino acids in SEQ ID NO:44. In certain embodiments, the numbering of the amino acids in a MBP is in reference to the numbering of the amino acids in SEQ ID NO:45. In certain embodiments, the numbering of the amino acids in a MBP is in reference to the numbering of the amino acids in SEQ ID NO:46.

In certain embodiments, the numbering of the amino acids in a PLP is in reference to the numbering of the amino acids in SEQ ID NO:39. In certain embodiments, the numbering of the amino acids in a PLP is in reference to the numbering of the amino acids in SEQ ID NO:47. In certain embodiments, the numbering of the amino acids in a PLP is in reference to the numbering of the amino acids in SEQ ID NO:48. In certain embodiments, the numbering of the amino acids in a PLP is in reference to the numbering of the amino acids in SEQ ID NO:49. In certain embodiments, the numbering of the amino acids in a PLP is in reference to the numbering of the amino acids in SEQ ID NO:50.

Nonlimiting examples of autoimmune diseases and disorders along with their corresponding unwanted antigens or a source of the unwanted antigens are illustrated in Table 1.

TABLE 1

| Autoimmune Disease or Disorder | Unwanted Antigen |
|---|---|
| autoimmune hemolytic anemia | Rh blood group antigens, I antigen |
| autoimmune thrombocytopenic | platelet integrin glycoprotein IIb:IIIa (GpIIb:IIIa) |
| purpura | (also known as integrin allbp3) |
| Goodpasture's syndrome | noncollagenous domain of basement membrane |
| | collagen type IV |
| pemphigus vulgaris | epidermal cadherin |
| rheumatoid arthritis | chondroitin sulfate proteoglycan l, synovial joint |
| | antigen, type II collagen, human cartilage-derived |
| | glycoprotein (HCgp39), stress-associated proteins, |
| | including grp78/BiP, citrullinated peptide antigens |
| | (ACPA), vimentin, fibrinogen, α-enolase, clusterin, |
| | histones, peptidyl arginine deiminase-4, RNA pol II |
| type 1 diabetes | islet cell antigens, islet-specific glucose-6-phosphatase |
| | catalytic subunit-related protein (IGRP), insulin, |
| | preproinsulin, tyrosine phosphatase like autoantigen, |
| | glutamic acid decarboxylase (GAD), |
| | carboxypeptidase H, tyrosine phosphatase-like |
| | proteins insulinoma antigen-2 (IA-2) and IA-2β (also |
| | termed phogrin or ICA512) |
| multiple sclerosis | myelin basic protein (MBP), proteolipid protein |
| | (PLP), myelin oligodendrocyte glycoprotein (MOG) |
| psoriasis, psoriasis vulgaris | skin antigens, keratin, keratinocyte-derived |
| | antimicrobial peptide cathelicidin (LL37), ADAMTS- |
| | like protein 5 (ADAMTSL5) that is produced by |
| | melanocytes |
| Grave's disease | thyroid-stimulating hormone receptor, thyrotropin |
| | receptor |
| Hashimoto's thyroiditis | thyroid antigens |
| Sjogren's disease | ribonucleoprotein antigens |
| systemic lupus erythematosus (SLE, | chromatin proteins, histones, ubiquitous |
| lupus) | ribonucleoprotein antigens |
| celiac disease | gluten proteins, including gliadin and glutenin |
| IgA nephropathy | IgA hinge region peptides |
| membranous nephropathy | alpha-glomerular basement membrane (GBM), |
| | PLA2R |
| scleroderma | Scl-70, centromere antigen, nucleolar antigens |
| primary biliary cirrhosis | PDL-E2 |
| atherosclerosis | HSP-60, HSP65, beta2- glycoproteinl (b2-GPl) |
| anca vasculitis | c-anca, PR3, p-anca, myeloperoxidase |
| inhibitor antibody formation in | factor VIII |
| hemophilia A and/or acquired | |
| hemophilia | |
| uveitis | MBP, esterase D (EsteD), esterase D and brain-type |
| | creatine kinase (BB-CK) |
| optic neuritis | MBP |
| myocarditis | α-myosin |
| uveitis and/or soluble antigen (S-Ag) | s-arrestin (retinal arrestin, S-Ag) |
| autoimmunity | |
| anti-MAG peripheral neuropathy | myelin-associated glycoprotein (MAG) |
| proteoglycan induced arthritis | CD4+ T cell epitope |
| (PGIA) or rheumatoid arthritis | |
| myasthenia gravis | acetylcholine receptor (AChR) or muscle specific |
| | kinase (MuSK) |

Nonlimiting examples of allergic diseases and disorders, allergic reactions along with corresponding allergens or a source of the corresponding allergans that can serve as an unwanted antigens are illustrated in Table 2*.

TABLE 2

| Allergic Disease, | |
|---|---|
| Disorder or Reaction | Unwanted Antigen |
| food allergy | peanuts, tree nuts, shellfish, |
| | eggs, milk, soy, wheat |
| asthma | dander (cat, etc.), pollens, |
| | dust-mite feces |
| seasonal rhinoconjunctivitis | pollens (ragweed, trees, grasses), |
| (hay fever) | dust-mite feces |
| acute urticaria (wheal-and-flare) | post-viral, animal hair, bee stings |
| systemic anaphylaxis | drugs, venoms, food |

*Additional allergenic antigens and sources useful as unwanted antigens of the application can be found, for example, in US 2015/0023992; US 2016/0251403; and US 2018/0078637, the content of each is incorporated herein by reference in its entirety.

Nonlimiting examples of antigens that can be used in accordance with the present invention for preventing or reducing rejection of a cell, tissue or organ after transplant into a subject include Y chromosome derived H-Y antigens and minor histocompatibility antigens HA-1-5.

As used herein, the terms “subsequence,” “fragment” or “portion” or the like refer to any portion of a larger sequence, up to and including the complete sequence. The minimum length of a subsequence is generally not limited, except that a minimum length of the subsequence of an unwanted antigen should be long enough to solicit an immune response (e.g., can act an immunogen). Polypeptide subsequences of the invention can be, for example, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 160, 170, 180, 190, or 200 amino acids or more in length.

As used herein, “extracellular domain” refers to a portion of a protein that is exposed on the extracellular side of a lipid bilayer of a cell.

As used herein, “transmembrane domain” refers to a portion of a protein that spans a lipid bilayer of a cell.

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide can be described herein according to the convention of providing the sequence in the 5′ to 3′ direction.

According to certain embodiments, the nucleic acid agent is a single-stranded (ssDNA) or a double-stranded DNA (dsDNA) molecule. According to certain embodiments, the nucleic acid agent is for therapeutic use, e.g., an ssDNA or dsDNA encoding a therapeutic transgene. According to certain embodiments, the dsDNA molecule is a minicircle, a nanoplasmid, open linear duplex DNA or a closed-ended linear duplex DNA (CELiD/ceDNA/doggybone DNA). According to certain embodiments, the ssDNA molecule is a closed circular or an open linear DNA.

As used herein, the terms “modify” and grammatical variations thereof, mean that a nucleic acid or protein deviates from a reference or parental sequence. A modified nucleic acid encoding a fusion protein, unwanted antigen, and/or leader sequence for cell secretion has been altered compared to a reference (e.g., wild-type) or parental nucleic acid. Modified nucleic acids can therefore have substantially the same, greater or less activity or function than a reference or parental nucleic acid, but at least retain partial activity, function and or sequence identity to the reference or parental nucleic acid. The modified nucleic acid can be genetically modified to encode a modified or variant fusion protein, unwanted antigen, and/or leader sequence for cell secretion.

A “modified nucleic acid encoding a fusion protein comprising an unwanted antigen and a leader sequence” means that the fusion protein, unwanted antigen, and/or leader sequence has alteration compared the parental unmodified nucleic acid encoding the fusion protein, unwanted antigen, and/or leader sequence. A particular example of a modification is a nucleotide substitution. The modified nucleic acid can also include a codon optimized nucleic acid that encodes the same protein as that of the wild-type protein or of the nucleic acid that has not been codon optimized. Codon optimization can be used in a broader sense, e.g., including removing CpGs, or introducing additional CpGs. The terms “modification” herein need not appear in each instance of a reference made to a nucleic acid encoding a fusion protein, unwanted antigen, and/or leader sequence.

In certain embodiments, for a modified nucleic acid encoding a fusion protein, unwanted antigen, and/or leader sequence, the fusion protein, unwanted antigen, and/or leader sequence retains at least part of a function or activity of wild-type or reference or parental fusion protein, unwanted antigen, and/or leader sequence.

As set forth herein, modified nucleic acids encoding a fusion protein, unwanted antigen, and/or leader sequence can exhibit different features or characteristics compared to a reference or parental nucleic acid. For example, modified nucleic acids include sequences with 100% identity to a reference nucleic acid encoding a fusion protein, unwanted antigen, and/or leader sequence as set forth herein, as well as sequences with less than 100% identity to a reference nucleic acid encoding a fusion protein, unwanted antigen, and/or leader sequence.

The terms “identity,” “homology,” and grammatical variations thereof, mean that two or more referenced entities are the same, when they are “aligned” sequences. Thus, by way of example, when two nucleic acids are identical, they have the same sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In certain embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In certain embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In certain embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

As set forth herein, modified nucleic acids encoding a fusion protein, unwanted antigen, and/or leader sequence can be distinct from or exhibit 100% identity or less than 100% identity to a reference nucleic acid encoding a fusion protein, unwanted antigen, and/or leader sequence.

Nucleic acids and expression cassettes can be delivered in order to affect the methods of the invention by any means in which the nucleic acid is transduced into and fusion protein expressed and subsequently secreted by a cell. A variety of ways of transducing cells with nucleic acids available. Such means include vectors, such as viral vectors, as well as lipids, nanoparticles, micelles and other formulations.

Recombinant cells capable of expressing a nucleic acid encoding a fusion protein of the invention can be used for delivery or administration.

Naked DNA such as minicircles and transposons can be used for administration or delivery or lentiviral vectors. Additionally, gene editing technologies such as zinc finger nucleases, meganucleases, TALENs, and CRISPR can also be used to deliver the coding sequence of the invention.

In certain embodiments, nucleic acids and expression cassettes of the invention are delivered as naked DNA, minicircles, transposons, of closed-ended linear duplex DNA.

In certain embodiments, nucleic acids, and expression cassettes of the invention are delivered or administered in AAV vector particles, or other viral particles, that are further encapsulated or complexed with liposomes, nanoparticles, lipid nanoparticles, polymers, microparticles, microcapsules, micelles, or extracellular vesicles.

In one embodiment, a nucleic acid encoding a fusion protein of the invention is delivered in a non-viral particle.

As used herein, a “non-viral vector” refers to a vector that does not contain the genetic information necessary for making a viral particle or a viral-like particle in a host cell alone or together with one or more appropriate helper vectors. As used herein, a “helper vector” refers to a vector that is able to mediate proper packaging of an expression cassette into a viral particle or a virus-like particle. The vector can be encapsulated, admixed, or otherwise associated with a non-viral delivery particle or nanoparticle.

Any suitable non-viral delivery system known to those skilled in the art in view of the present disclosure can be used in the invention in view of the present disclosure. A non-viral delivery particle or nanoparticle can be, for example, a lipid-based nanoparticle, a polymer-based nanoparticle, a protein-based nanoparticle, a microparticle, a microcapsule, a metallic particle-based nanoparticle, a peptide cage nanoparticle, a polyplex, a gold nanoparticle, a polymer-lipid hybrid nanoparticle, an inorganic nanoparticle, a carbon nanotube, a peptide-based nanoparticles, and other types of nanomaterials (see, Li et al., 2019, Wiley Interdiscip. Rev. Nanomed. Nanobiotechnol., 11(2): e1530. doi:10.1002/wnan.1530; Cullis et al., 2017, Mol. Ther., 25:1467-1475; Buck et al., 2019, ACS Nano, April 2, doi: 10.1021/acsnano.8b07858; Kaczmarek et al., 2017, Genome Med., 9:60, doi: 10.1186/s13073-017-0450-0; Zatsepin et al., 2016, Int. J. Nanomed., 11:3077-3086, doi: 10.2147/IJN.S106625; and Riley et al., 2017, Nanomaterials, 7:94, doi: 10.3390/nano7050094).

A non-viral delivery particle or nanoparticle of the instant invention can be constructed by any method known in the art in view of the present disclosure, and a non-viral vector of the instant invention comprising a nucleic acid molecule comprising a therapeutic transgene can be constructed by any method known in the art in view of the present disclosure.

Lipid-Based Delivery Systems

Lipid-based delivery systems are well known in the art, and any suitable lipid-based delivery system known to those skilled in the art in view of the present disclosure can be used in the invention. Examples of lipid-based delivery systems include, e.g., liposomes, lipid nanoparticles, micelles, or extracellular vesicles.

A “lipid nanoparticle” or “LNP” refers to a lipid-based vesicle useful for delivery of AAV and non-viral vectors having dimensions on the nanoscale. Examples of LNP can have a diameter of, for example, from about 10 nm to about 1000 nm, or from about 50 to about 500 nm, or from about 75 to about 127 nm. Without being bound by theory, the LNP is believed to provide the nucleic acid, expression cassette, or vector with partial or complete shielding from the immune system. Shielding allows delivery of the nucleic acid, expression cassette, or vector to a tissue or cell while avoiding inducing a substantial immune response against the nucleic acid, expression cassette, or vector in vivo. Shielding can also allow repeated administration without inducing a substantial immune response against the nucleic acid, expression vector, AAV vector, or non-viral vector in vivo (e.g., in a subject such as a human). Shielding can also improve or increase nucleic acid, expression cassette, or vector delivery efficiency in vivo.

The isoelectric point (pI) of AAV is in a pH range from about 6 to about 6.5. Thus, the AAV surface carries a slight negative charge. As such it can be beneficial for an LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, the disclosures of which are herein incorporated in their entirety.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids can also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids can include, without limitation, 1,2-dilinoleyloxy-N,N-dimethylaminopropane (DLinDMA), 1,2-dilinolenyloxy-N,N-dimethylaminopropane (DLenDMA), 1,2-di-y-linolenyloxy-N,N-dimethylaminopropane (g-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to, 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, y-DLen-C2K-DMA, and (DLin-MP-DMA) (also known as 1-B11).

Still further cationic lipids can include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ),1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1, 2-propanediol (DLinAP), 3-(N,N-dioleylamino)-1,2-propanedio (DOAP),1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOB A), 1,2-N,N′-dioleylcarbamyl-3-dimethylaminopropane (DOcarbDAP),1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECT AMINE® (comprising DOSPA and DOPE, available from GIBCO/BRL).

In certain embodiments, cationic lipid can be present in an amount from about 10% by weight of the LNP to about 85% by weight of the lipid nanoparticle, or from about 50% by weight of the LNP to about 75% by weight of the LNP.

Sterols can confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols can include campesterol, sitosterol, and stigmasterol. Sterols also include sterol-modified lipids, such as those described in U.S. Patent Application Publication 2011/0177156, the disclosure of which is herein incorporated in its entirety. In certain embodiments, a sterol can be present in an amount from about 5% by weight of the LNP to about 50% by weight of the lipid nanoparticle or from about 10% by weight of the LNP to about 25% by weight of the LNP.

LNP can comprise a neutral lipid. Neutral lipids can comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized by well-known techniques. In certain embodiments, lipids containing saturated fatty acids with carbon chain lengths in the range of C14 to C22 can be used. In another group of embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids can also be composed of sphingomyelin, dihydrosphingomyelin, or phospholipids with other head groups, such as serine and inositol.

In certain embodiments, the neutral lipid can be present in an amount from about 0.1% by weight of the lipid nanoparticle to about 75% by weight of the LNP, or from about 5% by weight of the LNP to about 15% by weight of the LNP.

LNP encapsulated nucleic acids, expression cassettes, and vectors can be incorporated into pharmaceutical compositions, e.g., a pharmaceutically acceptable carrier or excipient. Such pharmaceutical compositions are useful for, among other things, administration and delivery of LNP encapsulated acids, expression cassettes, and vectors to a subject in vivo or ex vivo.

Preparations of LNP can be combined with additional components. Non-limiting examples include polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, PEG can be a polyethylene glycol with an average molecular weight of about 550 to about 10,000 daltons and is optionally substituted by alkyl, alkoxy, acyl or aryl. In certain embodiments, the PEG can be substituted with methyl at the terminal hydroxyl position. In certain embodiments, the PEG can have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In certain embodiments, the terminal hydroxyl group can be substituted with a methoxy or methyl group.

PEG-modified lipids include the PEG-dialkyloxypropyl conjugates (PEG-DAA) described in U.S. Pat. Nos. 8,936,942 and 7,803,397, the disclosures of which are herein incorporated by reference in their entirety. PEG-modified lipids (or lipid-polyoxyethylene conjugates) that are useful can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, the disclosure of which is herein incorporated in its entirety, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.5% by weight of the LNP to about 20% by weight of the LNP, or from about 5% by weight of the LNP to about 15% by weight of the LNP.

Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In certain embodiments, prior to encapsulating LNPs can have a diameter in a range from about 10 nm to 500 nm, or from about 50 nm to about 200 nm, or from 75 nm to about 125 nm. In certain embodiments, LNP encapsulated nucleic acid, expression vector, AAV vector, or non-viral vector can have a diameter in a range from about 10 nm to 500 nm.

Polymer-Based Systems

Polymer-based delivery systems are well known in the art, and any suitable polymer-based delivery system or polymeric nanoparticle known to those skilled in the art in view of the present disclosure can be used in the invention. DNA can be entrapped into the polymeric matrix of polymeric nanoparticles or can be adsorbed or conjugated on the surface of the nanoparticles. Examples of commonly used polymers for gene delivery include, e.g., poly(lactic-co-glycolic acid) (PLGA), poly lactic acid (PLA), poly(ethylene imine) (PEI), chitosan, dendrimers, polyanhydride, polycaprolactone, and polymethacrylates.

The polymeric-based delivery systems for non-viral vectors can have different sizes, with diameters ranging from about 1 nm to about 1000 nm, optionally from about 10 nm to about 500 nm, optionally from about 50 nm to about 200 nm, optionally about 100 nm to about 150 nm, optionally about 150 nm or less.

Protein-Based Systems

Protein-based delivery systems are well known in the art, and any suitable protein-based delivery system or cell-penetrating peptide (CPP) known to those skilled in the art in view of the present disclosure can be used in the invention.

CPPs are short peptides (6-30 amino acid residues) that are potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25(1):1996-2006). Examples of CPPs include, e.g., cationic CPPs (highly positively charged) (e.g., the Tat peptide, penetratin, protamine, poly-L-lysine, polyarginine, etc.); amphipathic CPPs (chimeric or fused peptides, constructed from different sources, contain both positively and negatively charged amino acid sequences) (e.g., transportan, VT5, bactenecin-7 (Bac7), proline-rich peptide (PPR), SAP (VRLPPP)₃, TP10, pep-1, MPG, etc.); membranotropic CPPs (exhibit both hydrophobic and amphipathic nature simultaneously, and comprise both large aromatic residues and small residues) (e.g., gH625, SPIONs-PEG-CPP NPs, etc.); and hydrophobic CPPs (contain only non-polar motifs or residues) (e.g., SG3, PFVYLI, pep-7, fibroblast growth factors (FGF), etc.).

The protein-based delivery systems for non-viral vectors can have different sizes, with diameters ranging from about 1 nm to about 1000 nm, optionally from about 10 nm to about 500 nm, optionally from about 50 nm to about 200 nm, optionally about 100 nm to about 150 nm, optionally about 150 nm or less.

Peptide Cage Systems

Peptide cage-based delivery systems are well known in the art, and any suitable peptide cage-based delivery system known to those skilled in the art in view of the present disclosure can be used in the invention. In general, any proteinaceous material that is able to be assembled into a cage-like structure, forming a constrained internal environment, can be used. Several different types of protein “shells” can be assembled and loaded with different types of materials. For example, protein cages comprising a shell of viral coat protein(s) (e.g., from the Cowpea Chlorotic Mottle Virus (CCMV) protein coat) that encapsulate a non-viral material, as well as protein cages formed from non-viral proteins have been described (see, e.g., U.S. Pat. Nos. 6,180,389 and 6,984,386, U.S. Patent Application 20040028694, and U.S. Patent Application 20090035389, the disclosures of which are herein incorporated in their entirety). Peptide cages can comprise a proteinaceous shell that self-assembles to form a protein cage (e.g., a structure with an interior cavity which is either naturally accessible to the solvent or can be made to be so by altering solvent concentration, pH, equilibria ratios).

Examples of protein cages derived from non-viral proteins include, e.g., ferritins and apoferritins, derived from both eukaryotic and prokaryotic species, e.g., 12 and 24 subunit ferritins; and protein cages formed from heat shock proteins (HSPs), e.g., the class of 24 subunit heat shock proteins that form an internal core space, the small HSP of Methanococcus jannaschii, the dodecameric Dps HSP of E. coli, the MrgA protein, etc. As will be appreciated by those in the art, the monomers of the protein cages can be naturally occurring or variant forms, including amino acid substitutions, insertions and deletions (e.g., fragments) that can be made.

The protein cages can have different core sizes, with diameters ranging from about 1 nm to about 1000 nm, optionally from about 10 nm to about 500 nm, optionally from about 50 nm to about 200 nm, optionally about 100 nm to about 150 nm, optionally about 150 nm or less.

In particular embodiments, an expression cassette, such as an mRNA, encoding a fusion protein of the invention is delivered in a non-viral particle. In further embodiments, an expression cassette, such as an mRNA, encoding a fusion protein of the invention is delivered in a lipid nanoparticle.

In one embodiment, an expression cassette is comprised within a vector. Nonlimiting examples of vectors include viral vectors such as lentiviral, adenoviral and adeno-associated viral (AAV) vectors.

As used herein, the term “vector” refers to small carrier nucleic acid molecule, a plasmid, virus, or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell.

A vector nucleic acid sequence generally contains at least an origin of replication for propagation in a cell and optionally additional elements, such as a nucleic acid (e.g., nucleic acid encoding a fusion protein), expression control element (e.g., a promoter, enhancer), intron, an inverted terminal repeat (ITR), selectable marker (e.g., antibiotic resistance), polyadenylation signal.

A viral vector is derived from or based upon one or more nucleic acid elements that comprise a viral genome. Particular viral vectors include adeno-associated virus (AAV) and lentiviral vectors.

The term “recombinant,” as a modifier of vector, as well as a modifier of an amino acid or nucleic acid sequences, means that the compositions have been manipulated or engineered in a fashion that generally does not occur in nature. A particular example of a recombinant AAV (rAAV) vector would be where a nucleic acid sequence that is not normally present in the wild-type AAV genome is inserted within the AAV genome. Although the term “recombinant” is not always used herein in reference to AAV vectors, as well as sequences such as nucleic acids, recombinant forms including nucleic acids encoding fusion proteins as set forth herein, are expressly included in spite of any such omission.

A “recombinant AAV vector” or “rAAV” is derived from the wild type genome of AAV by using molecular methods to remove the wild type genome from the AAV genome, and replacing with a non-native nucleic acid sequence. Typically, for AAV one or both inverted terminal repeat (ITR) sequences of AAV genome are retained in the AAV vector. rAAV is distinguished from an AAV genome, since all or a part of the AAV genome has been replaced with a non-native (non-AAV) sequence with respect to the AAV genomic nucleic acid. Incorporation of a non-native sequence therefore defines the AAV vector as a “recombinant” vector, which can be referred to as a “rAAV vector.”

A rAAV vector genome can be packaged—referred to herein as a “particle”—for subsequent infection (transduction) of a cell, ex vivo, in vitro or in vivo. Where a recombinant AAV vector genome is encapsidated or packaged into an AAV particle, the particle can also be referred to as a “rAAV vector” or “rAAV particle.” Such rAAV particles include proteins that encapsidate or package the vector genome and in the case of AAV, they are referred to as capsid proteins.

A “vector genome” or conveniently abbreviated as “vg” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid can be referred to as the “plasmid backbone,” which is important for cloning and amplification of the plasmid, a process that is needed for propagation and recombinant virus production, but is not itself packaged or encapsidated into virus (e.g., AAV) particles. Thus, a “vector genome” refers to the nucleic acid that is packaged or encapsidated by virus (e.g., AAV).

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes).

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors/particles include any viral strain or serotype. As a non-limiting example, an AAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rh10, AAV3B or AAV-2i8, for example. Such AAV vectors/particles can be based on the same strain or serotype (or subgroup or variant) or be different from each other. As a non-limiting example, a rAAV vector genome or particle (capsid) based upon one serotype genome can be identical to one or more of the capsid proteins that package the vector. In addition, a rAAV vector genome can be based upon an AAV serotype genome distinct from one or more of the capsid proteins that package the vector genome, in which case at least one of the three capsid proteins could be a different AAV serotype, e.g., AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2), or variant thereof, for example. More specifically, a rAAV2 vector genome can comprise AAV2 ITRs but capsids from a different serotype, such as AAV1, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2), or variant thereof, for example. Accordingly, rAAV vectors include gene/protein sequences identical to gene/protein sequences characteristic for a particular serotype, as well as “mixed” serotypes, which also can be referred to as “pseudotypes.”

In various exemplary embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2) capsid proteins (VP1, VP2, and/or VP3 sequences). In various exemplary embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10, AAV3B, or AAV-2i8, ITR(s).

In particular embodiments, rAAV vectors/particles include AAV1, AAV2, AAV3, AAV4, AAV5, AAV6, AAV7, AAV8, AAV9, AAV10, AAV11, AAV12, Rh10, Rh74, AAV3B, and AAV-2i8 variants (e.g., capsid variants, such as amino acid insertions, additions, substitutions and deletions and ITR nucleotide insertions, additions, substitutions and deletions in the context of a rAAV vector) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV particles, such as AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2) and variants, hybrids and chimeric sequences, can be constructed using recombinant techniques that are known to a skilled artisan, to include one or more nucleic acid sequences (transgenes) flanked with one or more functional AAV ITR sequences at the 5′ and/or 3′ end. rAAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

Host cells for producing recombinant AAV particles include but are not limited to microorganisms, yeast cells, insect cells, and mammalian cells that can be, or have been, used as recipients of rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Other host cell lines appropriate for recombinant AAV production are described in International Application PCT/2017/024951.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

Methods of generating recombinant AAV vectors/particles capable of transducing mammalian cells are known in the art. For example, recombinant AAV vectors/particles can be produced as described in U.S. Pat. No. 9,408,904; and International Applications PCT/US2017/025396 and PCT/US2016/064414.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides (e.g., nucleic acids encoding fusion proteins).

Nucleic acids such as vector genome, cDNA, genomic DNA, RNA, and fragments thereof can be single, double, or triplex, linear or circular, and can be of any length. In discussing nucleic acids, a sequence or structure of a particular nucleic acid may be described herein according to the convention of providing the sequence in the 5′ to 3′ direction.

The term “transduce” and grammatical variations thereof refer to introduction of a molecule such as an rAAV vector into a cell or host organism, such as a plurality of cells in the subject to which the vector is administered. The nucleic acid may or may not be integrated into genomic nucleic acid of the recipient cell. The introduced nucleic acid may also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation, for example, of a nucleic acid (e.g., a transgene) into the cell. Thus, a “transduced” cell is a cell into which, or a progeny thereof in which an exogenous nucleic acid (e.g., nucleic acid encoding a fusion protein) has been introduced. The cell(s) can be propagated and the introduced protein expressed. For gene therapy uses and methods, a transduced cell can be in a subject, such as a mammal, a primate, or a human.

The term “expression cassette”, as used herein, refers to a nucleic acid construct comprising nucleic acid elements sufficient for the expression of the nucleic acid molecule of the instant invention. Typically, an expression cassette comprises the nucleic acid molecule of the instant invention operably linked to a promoter sequence.

An “expression control element” refers to nucleic acid sequence(s) that influence expression of an operably linked nucleic acid. Expression control elements as set forth herein include promoters and enhancers. Vector sequences including AAV vectors can include one or more “expression control elements.” Typically, such elements are included to facilitate proper nucleic acid transcription and as appropriate translation (e.g., a promoter, enhancer, splicing signal for introns, maintenance of the correct reading frame of the gene to permit in-frame translation of mRNA and, stop codons, etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of AAV vectors, expression control elements will typically be within 1 to 1000 nucleotides from the transcription start site of the nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

An “enhancer” as used herein can refer to a sequence that is located adjacent to the nucleic acid encoding a fusion protein. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a nucleic acid sequence encoding a fusion protein. Enhancer elements typically increase expressed of an operably linked nucleic acid above expression afforded by a promoter element.

An expression construct or cassette may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

Examples of promoters that are active in liver are the transthyretin (TTR) gene promoter; human alpha 1-antitrypsin (hAAT) promoter; albumin, Miyatake, et al., J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996), among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked nucleic acid. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Exemplary nonlimiting examples of expression control elements include an ApoE/hAAT enhancer/promoter sequence, a CAG (SEQ ID NO:3) promoter, cytomegalovirus (CMV) immediate early promoter/enhancer, Rous sarcoma virus (RSV) promoter/enhancer, SV40 promoter, dihydrofolate reductase (DHFR) promoter, and a chicken β-actin (CBA) promoter.

Expression control elements also include the native elements(s). A native control element (e.g., promoter) may be used when it is desired that expression of the nucleic acid should mimic the native expression. The native element may be used when expression of the nucleic acid is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g., promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a nucleic acid sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

Nucleic acids, expression cassettes, expression vectors (e.g., AAV vector genomes), plasmids, including nucleic acids encoding fusion proteins may be prepared by using recombinant DNA technology methods. The availability of nucleotide sequence information enables preparation of isolated nucleic acid molecules of the invention by a variety of means. Nucleic acids encoding fusion proteins and expression cassettes containing such nucleic acids can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids may be maintained as DNA in any convenient cloning vector. In a one embodiment, clones are maintained in a plasmid cloning/expression vector, such as pBluescript (Stratagene, La Jolla, Calif.), which is propagated in a suitable E. coli host cell. Alternatively, nucleic acids may be maintained in vector suitable for expression in mammalian cells, for example, an AAV vector. In cases where post-translational modification affects protein function, nucleic acid molecule can be expressed in mammalian cells.

As disclosed herein, rAAV vectors may optionally comprise regulatory elements necessary for expression of the nucleic acid in a cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Methods and uses of the invention include delivering (transducing) nucleic acid into host cells, including dividing and/or non-dividing cells. The nucleic acids, expression cassettes, vectors, methods, uses and pharmaceutical formulations of the invention are additionally useful in a method of delivering, administering or providing a fusion protein encoded by a nucleic acid to a subject in need thereof, as a method of treatment. In this manner, the nucleic acid is transcribed, fusion protein expressed and secreted by cells in vivo in a subject.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats) and experimental animals (mouse, rat, rabbit, guinea pig). Human subjects include fetal, neonatal, infant, juvenile and young adult subjects. Subjects include animal disease models, for example, mouse and other animal models of autoimmune diseases and disorders such as the EAE model and models of allergies, allergic diseases and disorders.

Subjects appropriate for treatment in accordance with the invention include those having or at risk of having an autoimmune disease or disorder, or an allergy or allergic disease or disorder. Subjects can be tested for an autoimmune disease or disorder or an allergy or allergic disease or disorder to determine if such subjects are appropriate for treatment according to methods of the invention. Subjects appropriate for treatment in accordance with the invention also include those subjects that would benefit from immune suppression. Treated subjects can be monitored after treatment periodically, e.g., every 1-4 weeks, 1-6 months, 6-12 months, or 1, 2, 3, 4, 5 or more years.

Subjects can be tested for an immune response against a self-antigen, autoantigen, allergen, allergenic antigen or allogenic antigens, e.g., antibodies against such antigens or allergen. Candidate subjects can therefore be screened prior to treatment according to a method of the invention.

Subjects also can be tested for antibodies against AAV before, during or after treatment, and optionally monitored for a period of time after treatment. Subjects that have, are at risk of developing AAV antibodies or have developed AAV antibodies can be treated with an immunosuppressive agent, or other regimen as set forth herein.

Subjects appropriate for treatment in accordance with the invention that have developed AAV antibodies or are at risk of developing antibodies against AAV. rAAV vectors can be administered or delivered to such subjects using several techniques. For example, AAV empty capsid (i.e., AAV lacking vector genome) can be delivered to bind to the AAV antibodies in the subject thereby allowing the rAAV vector comprising the nucleic acid to transduce cells of the subject.

As set forth herein, rAAV are useful as gene therapy vectors as they can penetrate cells and introduce nucleic acid/genetic material into the cells. Because AAV are not associated with pathogenic disease in humans, rAAV vectors are able to deliver nucleic acid sequences (e.g., fusion proteins) to human patients without causing substantial AAV pathogenesis or disease.

rAAV vectors possess a number of desirable features for such applications, including tropism for dividing and non-dividing cells. Early clinical experience with these vectors also demonstrated no sustained toxicity and immune responses are typically minimal or undetectable. AAV are known to infect a wide variety of cell types in vivo by receptor-mediated endocytosis or by transcytosis. These vector systems have been tested in humans targeting many tissues, such as liver, skeletal muscle, airways, joints and hematopoietic stem cells.

It may be desirable to introduce a rAAV vector that can provide, for example, multiple copies of nucleic acid encoding a fusion protein and hence greater amounts of fusion protein. Improved rAAV vectors and methods for producing these vectors have been described in detail in a number of references, patents, and patent applications, including: Wright J. F. (Hum Gene Ther 20:698-706, 2009).

rAAV vectors can be administered to a patient in a biologically compatible carrier, for example, via intravenous injection or infusion. rAAV vectors may be administered alone or in combination with other molecules. Accordingly, expression cassettes, vectors and other compositions, small organic molecules, drugs, biologics (e.g., immunosuppressive agents) can be incorporated into pharmaceutical compositions. Such pharmaceutical compositions are useful for, among other things, administration and delivery to a subject in vivo or ex vivo.

In particular embodiments, a pharmaceutical composition comprises more than one nucleic acid, expression vector, viral particle, lenti-viral particle, and/or rAAV particle in a biologically compatible carrier or excipient.

In particular embodiments, pharmaceutical compositions also contain a pharmaceutically or biologically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, expression cassette, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technomic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosuppressive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment of a disease typically are effective to provide a response to one, multiple or all adverse symptoms, consequences or complications of the disease, one or more adverse symptoms, disorders, illnesses, pathologies, or complications, caused by or associated with the disease, to a measurable extent, although decreasing, reducing, inhibiting, suppressing, limiting or controlling progression or worsening of the disease is a satisfactory outcome. The treatment can be to ameliorate or to provide a therapeutic benefit or improvement of the disease.

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of nucleic acid encoding fusion protein expression required to achieve a therapeutic effect, the specific disease treated, any host immune response to the viral vector and the stability of the protein expressed.

In certain embodiments, AAV doses will be greater than about 1.5×10¹¹ recombinant AAV vector genomes/kg. For example, a dose of about 2×10¹¹recombinant AAV vector genomes/kg or greater than about 2×10¹¹ recombinant AAV vector genomes/kg; a dose of about 3×10¹¹recombinant AAV vector genomes/kg or greater than about 3×10¹¹ recombinant AAV vector genomes/kg; a dose of about 4×10¹¹recombinant AAV vector genomes/kg or greater than about 4×10¹¹ recombinant AAV vector genomes/kg; a dose of about 5×10¹¹recombinant AAV vector genomes/kg or greater than about 5×10¹¹ recombinant AAV vector genomes/kg; a dose of about 1×10¹² recombinant AAV vector genomes/kg or greater than about 1×10¹² recombinant AAV vector genomes/kg; a dose of about 2×10¹² recombinant AAV vector genomes/kg or greater than about 2×10¹² recombinant AAV vector genomes/kg; a dose of about 3×10¹² recombinant AAV vector genomes/kg or greater than about 3×10¹² recombinant AAV vector genomes/kg; a dose of about 4×10¹² recombinant AAV vector genomes/kg or greater than about 4×10¹² recombinant AAV vector genomes/kg; a dose of about 5×10¹² recombinant AAV vector genomes/kg or greater than about 5×10¹² recombinant AAV vector genomes/kg.

In certain embodiments, AAV doses will be greater than about 1.5×10¹³ recombinant AAV vector genomes/kg. For example, a dose of about 5×10¹³ recombinant AAV vector genomes/kg or greater than about 5×10¹³ recombinant AAV vector genomes/kg; a dose of about 1×10¹⁴ recombinant AAV vector genomes/kg or greater than about 1×10¹⁴ recombinant AAV vector genomes/kg; a dose of about 5×10¹⁴ recombinant AAV vector genomes/kg or greater than about 5×10¹⁴ recombinant AAV vector genomes/kg; a dose of about 1×10¹⁵ recombinant AAV vector genomes/kg or greater than about 1×10¹⁵ recombinant AAV vector genomes/kg; and a dose of about 5×10¹⁵ recombinant AAV vector genomes/kg or greater than about 5×10¹⁵ recombinant AAV vector genomes/kg.

Exemplary dose ranges of recombinant AAV vector genomes/kg administered are a dose range from about 1.5×10¹¹ to about 5×10¹³ recombinant AAV vector genomes/kg; a dose range from about 1.5×10¹¹ to about 2×10¹¹ recombinant AAV vector genomes/kg; a dose range from about 2×10¹¹ to about 2.5×10¹¹ recombinant AAV vector genomes/kg; a dose range from about 2.5×10¹¹ to about 3×10¹¹ recombinant AAV vector genomes/kg; a dose range from about 3×10¹¹ to about 3.5×10¹¹ recombinant AAV vector genomes/kg; a dose range from about 3.5×10¹¹ to about 4×10¹¹ recombinant AAV vector genomes/kg; a dose range from about 4×10¹¹ to about 4.5×10¹¹ recombinant AAV vector genomes/kg; a dose range from about 4.5×10¹¹ to about 5×10¹¹ recombinant AAV vector genomes/kg; a dose range from about 5×10¹¹ to about 1×10¹² recombinant AAV vector genomes/kg; a dose range from about 1×10¹² to about 1.5×10¹² recombinant AAV vector genomes/kg; a dose range from about 1.5×10¹² to about 2×10¹² recombinant AAV vector genomes/kg; a dose range from about 2×10¹² to about 2.5×10¹² recombinant AAV vector genomes/kg; a dose range from about 2.5×10¹² to about 3×10¹² recombinant AAV vector genomes/kg; a dose range from about 3×10¹² to about 3.5×10¹² recombinant AAV vector genomes/kg; a dose range from about 3.5×10¹² to about 4×10¹² recombinant AAV vector genomes/kg; a dose range from about 4×10¹² to about 4.5×10¹² recombinant AAV vector genomes/kg; a dose range from about 4.5×10¹² to about 5×10¹² recombinant AAV vector genomes/kg; and a dose range from about 5×10¹² to about 1×10¹³ recombinant AAV vector genomes/kg.

Exemplary dose ranges of recombinant AAV vector genomes/kg administered are a dose range from about 1.5×10¹³ to about 5×10¹⁵ recombinant AAV vector genomes/kg; a dose range from about 1×10¹⁴ to about 3×10¹⁵ recombinant AAV vector genomes/kg; a dose range from about 2×10¹⁴ to about 2×10¹⁵ recombinant AAV vector genomes/kg; a dose range from about 2.5×10¹⁴ to about 7.5×10¹⁴ recombinant AAV vector genomes/kg; a dose range from about 5×10¹⁴ to about 5×10¹⁵ recombinant AAV vector genomes/kg; and a dose range from about 1×10¹⁵ to about 5×10¹⁵ recombinant AAV vector genomes/kg.

In certain embodiments, AAV vector genomes/kg are administered at a dose of about 1×10¹¹ vector genomes/kg, administered at a dose of about 2×10¹¹ vector genomes/kg, administered at a dose of about 3×10¹¹ vector genomes/kg, administered at a dose of about 4×10¹¹ vector genomes/kg, administered at a dose of about 5×10¹¹ vector genomes/kg, administered at a dose of about 6×10¹¹ vector genomes/kg, administered at a dose of about 7×10¹¹ vector genomes/kg, administered at a dose of about 8×10¹¹ vector genomes/kg, administered at a dose of about 9×10¹¹ vector genomes/kg, administered at a dose of about 1×10¹² vector genomes/kg, administered at a dose of about 2×10¹² vector genomes/kg, administered at a dose of about 3×10¹² vector genomes/kg, administered at a dose of about 4×10¹² vector genomes/kg, administered at a dose of about 5×10¹² vector genomes/kg, administered at a dose of about 6×10¹² vector genomes/kg, administered at a dose of about 7×10¹² vector genomes/kg, administered at a dose of about 8×10¹² vector genomes/kg, administered at a dose of about 9×10¹² vector genomes/kg, administered at a dose of about 1×10¹³ vector genomes/kg, administered at a dose of about 2×10¹³ vector genomes/kg, administered at a dose of about 3×10¹³ vector genomes/kg, administered at a dose of about 4×10¹³ vector genomes/kg, administered at a dose of about 5×10¹³ vector genomes/kg, administered at a dose of about 6×10¹³ vector genomes/kg, administered at a dose of about 7×10¹³ vector genomes/kg, administered at a dose of about 8×10¹³ vector genomes/kg, administered at a dose of about 9×10¹³ vector genomes/kg.

In certain embodiments, AAV vector genomes/kg are administered at a dose of about 1×10¹⁴ vector genomes/kg, administered at a dose of about 2×10¹⁴ vector genomes/kg, administered at a dose of about 3×10¹⁴ vector genomes/kg, administered at a dose of about 4×10¹⁴ vector genomes/kg, administered at a dose of about 5×10¹⁴ vector genomes/kg, administered at a dose of about 6×10¹⁴ vector genomes/kg, administered at a dose of about 7×10¹⁴ vector genomes/kg, administered at a dose of about 8×10¹⁴ vector genomes/kg, administered at a dose of about 9×10¹⁴ vector genomes/kg, administered at a dose of about 1×10¹⁵ vector genomes/kg, administered at a dose of about 2×10¹⁵ vector genomes/kg, administered at a dose of about 3×10¹⁵ vector genomes/kg, administered at a dose of about 4×10¹⁵ vector genomes/kg, or administered at a dose of about 5×10¹⁵ vector genomes/kg.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, and pharmaceutical compositions thereof can be packaged in single or multiple unit dosage form for ease of administration and uniformity of dosage.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of nucleic acid encoding a fusion protein for treatment of an autoimmune disease or disorder, an allergy or allergic disease or disorder or transplant rejection.

Accordingly, methods and uses of the invention also include, among other things, methods and uses that result in a reduced need or use of another compound, agent, drug, therapeutic regimen, treatment protocol, process, or remedy. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

Administration or in vivo delivery to a subject can be performed after or prior to development of an adverse symptom, condition, complication, etc. caused by or associated with the autoimmune disease or disorder or allergy or allergic disease or disorder. For example, a screen (e.g., genetic) can be used to identify such subjects as candidates for invention compositions, methods and uses.

Administration or in vivo delivery to a subject in accordance with the methods and uses of the invention as disclosed herein can be practiced within 1-2, 2-4, 4-12, 12-24 or 24-72 hr after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

Therapeutic doses will depend on, among other factors, the age and general condition of the subject, the severity of the disease or disorder. A therapeutically effective amount in humans will fall in a relatively broad range that may be determined by a medical practitioner based on the response of an individual patient.

Compositions such as pharmaceutical compositions may be delivered to a subject, so as to allow production of the encoded protein. In a particular embodiment, pharmaceutical compositions comprise sufficient genetic material to enable a recipient to produce a therapeutically effective amount of a protein in the subject.

Compositions may be formulated and/or administered in any sterile, biocompatible pharmaceutical carrier, including, but not limited to, saline, buffered saline, dextrose, and water. The compositions may be formulated and/or administered to a patient alone, or in combination with other agents (e.g., co-factors) which influence hemostasis.

Methods and uses of the invention include delivery and administration systemically, regionally or locally, or by any route, for example, by injection or infusion. Delivery of the pharmaceutical compositions in vivo may generally be accomplished via injection. For example, rAAV vectors/particles may be administered intravenously.

Invention cassettes, compositions, vectors, methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (e.g., immunosuppressive agents), small organic compounds (e.g., immunosuppressive agents) and drugs. Such biologics (e.g., immunosuppressive agents), small organic compounds, drugs, treatments and therapies can be administered or performed prior to, substantially contemporaneously with or following any other method or use of the invention. In certain embodiments, an invention cassette, composition, vector, method or use is combined with one of more of beta interferon, ocrelizumab, glatiramer acetate, dimethyl fumarate, fingolimod, teriflunomide, natalizumab, alemtuzumab, or mitoxantrone, and in particular embodiments for the treatment of multiple sclerosis.

The compound, agent, drug, treatment or other therapeutic regimen or protocol can be administered as a combination composition, or administered separately, such as concurrently or in series or sequentially (prior to or following) delivery or administration of a nucleic acid, expression cassette, vector, or rAAV particle. The invention therefore provides combinations in which a method or use of the invention is in a combination with at least one of any compound, agent, drug, therapeutic regimen, treatment protocol, process, remedy or composition, set forth herein or known to one of skill in the art. The compound, small organic molecule, drug, therapeutic regimen, treatment protocol, process, remedy or composition can be administered or performed prior to, substantially contemporaneously with or following administration of a nucleic acid, expression cassette, vector, or rAAV particle of the invention, to a subject.

In certain embodiments, an immunosuppressive agent is administered.

In certain embodiments, an immunosuppressive agent is an anti-inflammatory agent.

In certain embodiments, an immunosuppressive agent is a steroid, e.g., a corticosteroid. In certain embodiments, an immunosuppressive agent is prednisone, prednisolone, calcineurin inhibitor (e.g., cyclosporine, tacrolimus), CD52 inhibitor (e.g., alemtuzumab), CTLA4-Ig (e.g., abatacept, belatacept), anti-CD3 mAb, anti-LFA-1 mAb (e.g., efalizumab), anti-CD40 mAb (e.g., ASKP1240), anti-CD22 mAb (e.g., epratuzumab), anti-CD20 mAb (e.g., rituximab, orelizumab, ofatumumab, veltuzumab), proteasome inhibitor (e.g., bortezomib), TACI-Ig (e.g., atacicept), anti-05 mAb (e.g., eculizumab), mycophenolate, azathioprine, sirolimus everolimus, TNFR-Ig, anti-TNF mAb, tofacitinib, anti-IL-2R (e.g., basiliximab), anti-IL-17 mAb (e.g., secukinumab), anti-IL-6 mAb (e.g., anti-IL-6 antibody sirukumab, anti-IL-6 receptor antibody tocilizumab (Actemra®), IL-10, TGF-beta, a B cell targeting antibody (e.g., rituximab), a mammalian target of rapamycin (mTOR) inhibitor (e.g., rapamycin), synthetic vaccine particle (SVP™)-rapamycin (rapamycin encapsulated in a biodegradable nanoparticle), intravenous gamma globulin (IVIG), omalizumab, methotrexate, a tyrosine kinase inhibitor (e.g., ibrutinib), cyclophosphamide, fingolimod, an inhibitor of B-cell activating factor (BAFF) (e.g, anti-BAFF mAb, e.g., belimumab), an inhibitor of a proliferation-inducing ligand (APRIL), anti-IL-1b mAb (e.g., canakinumab (Haris®)), a C3a inhibitor, a Tregitope (see, e.g., U.S. Pat. No. 10,213,496), or a combination and/or derivative thereof, and/or administration of one or more immunosuppressive protocol or procedure, such as B-cell depletion, immunoadsorption, and plasmapheresis.

In certain embodiments, an agent to induce/increase levels of IDO (indoleamine 2,3-dioxygenase) is administered in combination (before, concomitantly with, or after) with a nucleic acid, expression cassette, vector, or rAAV particle of the invention, to a subject. Agents to induce/increase levels of IDO include, for example and without limitation, an IDO encoding nucleic acid, including, for example and without limitation, an IDO encoding mRNA.

IDO is a potent immunosuppressive enzyme that can inhibit T-cell responses and induce T-cell apoptosis by regulation of tryptophan metabolism, thereby inducing tolerance. IDO has been shown to be important to fetal development, preventing the immune system of the mother from rejecting the fetus. Increased expression of IDO is associated with cancer and shutting it down is a focus in many oncologic clinical trials (see Prendergast et al., 2017 Cancer Res., 77:6795-6811). Adenovirus delivery of IDO has been shown to improve renal function and morphology following allogeneic (non-MCH/HLA matched) kidney transplantation in rats (Vavrincova-Yaghi et al., 2011, J Gene Med, 13:373-81), and to attenuate chronic transplant dysfunction (CTD; primary cause of late allograft loss in kidney transplantation), also in rats (Vavrincova-Yaghi et al., 2016, Gene Ther., 23:797-806). Transposon-based co-delivery of genes encoding FVIII and IDO has been shown to attenuate inhibitor development in gene therapy treated Hem A mice (Liu et al., 2009, Gene Ther., 16:724-733).

In certain embodiments, the compositions and methods of the present invention are used in combination with immune suppression protocols. Strategies to overcome or avoid humoral immunity to AAV in systemic gene transfer include, administering high vector doses, use of AAV empty capsids as decoys to adsorb anti-AAV antibodies, administration of immunosuppressive drugs to decrease, reduce, inhibit, prevent or eradicate the humoral immune response to AAV, changing the AAV capsid serotype or engineering the AAV capsid to be less susceptible to neutralizing antibodies (NAb), use of plasma exchange cycles to adsorb anti-AAV immunoglobulins, thereby reducing anti-AAV antibody titer, and use of delivery techniques such as balloon catheters followed by saline flushing. Such strategies are described in Mingozzi et al., 2013, Blood, 122:23-36. Additional strategies include use of AAV-specific plasmapheresis columns to selectively deplete anti-AAV antibodies without depleting the total immunoglobulin pool from plasma, as described in Bertin et al., 2020, Sci. Rep. 10:864. Apheresis strategies to remove, deplete, capture, and/or inactivate AAV antibodies in subjects are described in WO2019018439.

In certain embodiments, the nucleic acids, expression cassettes, vectors, or rAAV particles of the invention are used in combination with methods to reduce antibody (e.g., IgG) levels in human plasma. In certain embodiments, the nucleic acids, expression cassettes, vectors, or rAAV particles of the invention are used in combination with an endopeptidase (e.g., IdeS from Streptococcus pyogenes) or a modified variant thereof, or an endoglycosidase (e.g., S. pyogenes EndoS) or a modified variant thereof. In certain embodiments nucleic acids, expression cassettes, vectors, or rAAV particles of the invention are administered to a subject in combination with an endopeptidase (e.g., IdeS from Streptococcus pyogenes) or a modified variant thereof, or an endoglycosidase (e.g., EndoS from S. pyogenes) or a modified variant thereof to reduce or clear neutralizing antibodies against AAV capsid and enable treatment of patients previously viewed as not eligible for gene therapy or that develop AAV antibodies after AAV gene therapy. Such strategies are described in Leborgne et al., C., Barbon, E., Alexander, J. M. et al., 2020, Nat. Med., 26:1096-1101 (2020), doi.org/10.1038/s41591-020-0911-7.

Methods and uses of the invention include delivery and administration systemically, regionally or locally, or by any route, for example, by injection or infusion. Delivery of the pharmaceutical compositions in vivo can generally be accomplished via injection using a conventional syringe, although other delivery methods such as convection-enhanced delivery are envisioned (See e.g., U.S. Pat. No. 5,720,720, the disclosure of which is herein incorporated in its entirety). For example, compositions can be delivered subcutaneously, epidermally, intradermally, intrathecally, intraorbitally, intramucosally, intranasally, intraperitoneally, intravenously, intra-pleurally, intraarterially, intracavitary, orally, intrahepatically, via the portal vein, or intramuscularly. Other modes of administration include oral and pulmonary administration, suppositories, and transdermal applications. Depending on the therapeutic indication, a clinician specializing in the treatment of patients can determine the optimal route for administration of AAV vectors and non-viral vectors based on a number of criteria, including, but not limited to the condition of the patient and the purpose of the treatment.

Exemplary ratio of AAV empty capsids to the rAAV particles can be within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced or predicted to be produced in a particular subject.

AAV antibodies may be preexisting and may be present at levels that reduce or block rAAV transduction of target cells. Alternatively, AAV antibodies may develop after exposure to AAV or administration of an rAAV vector. If such antibodies develop after administration of an rAAV vector, these subjects can also be treated accordingly.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., nucleic acid encoding a fusion protein, expression cassettes comprising a nucleic acid encoding fusion protein, vectors and rAAV particles comprising nucleic acids encoding fusion proteins) are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 86% or more identity, includes 86%, 87%, 88%, 89%, 90%, 91%, 92%, 93%, 94%, 95%, 96%, 97%, 98%, 99%, 100%, etc., as well as 86.1%, 86.2%, 86.3%, 86.4%, 86.5%, etc., 87.1%, 88.2%, 88.3%, 88.4%, 88.5%, etc., and so forth.

Reference to an integer with more (greater) or less than includes any number greater or less than the reference number, respectively. Thus, for example, a reference to greater than 1.5×10¹³, includes 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, etc.

As used herein, all numerical values or ranges include sub ranges and fractions of the values and integers within such ranges and sub ranges and the wrong 1 as well as the file okay thanks fractions of the integers within such ranges unless the context clearly indicates otherwise. Thus, to illustrate, reference to a numerical range, such as 1-10 includes 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 3-10, etc.; and 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

A Secretable Form of Myelin Oligodendrocyte Glycoprotein (MOG)

Mus musculus full-length MOG cDNA (with native leader) (SEQ ID NO:4):

ATGGCCTGTTTGTGGAGCTTCTCTTGGCCCAGCTGCTTCCTCTCCCTTC
TCCTCCTCCTTCTCCTCCAGTTGTCATGCAGCTATGCAGGACAATTCAG
AGTGATAGGACCAGGGTATCCCATCCGGGCTTTAGTTGGGGATGAAGCA
GAGCTGCCGTGCCGCATCTCTCCTGGGAAAAATGCCACGGGCATGGAGG
TGGGTTGGTACCGTTCTCCCTTCTCAAGAGTGGTTCACCTCTACCGAAA
TGGCAAGGACCAAGATGCAGAGCAAGCACCTGAATACCGGGGACGCACA
GAGCTTCTGAAAGAGACTATCAGTGAGGGAAAGGTTACCCTTAGGATTC
AGAACGTGAGATTCTCAGATGAAGGAGGCTACACCTGCTTCTTCAGAGA
CCACTCTTACCAAGAAGAGGCAGCAATGGAGTTGAAAGTGGAAGATCCC
TTCTATTGGGTCAACCCCGGTGTGCTGACTCTCATCGCACTTGTGCCTA
CGATCCTCCTGCAGGTCTCTGTAGGCCTTGTATTCCTCTTCCTGCAGCA
CAGACTGAGAGGAAAACTTCGTGCAGAAGTAGAGAATCTCCATCGGACT
TTTGATCCTCACTTCCTGAGGGTGCCCTGCTGGAAGATAACACTGTTTG
TTATTGTGCCTGTTCTTGGACCCCTGGTTGCCTTGATCCTGTGCTACAA
CTGGCTGCACCGAAGACTGGCAGGACAGTTTCTTGAAGAGCTAAGAAAC
CCCTTTTGAAGATC

Mus musculus full-length MOG cDNA (with native leader; start to stop) (SEQ ID NO:461):

ATGGCCTGTTTGTGGAGCTTCTCTTGGCCCAGCTGCTTCCTCTCCCTTC
TCCTCCTCCTTCTCCTCCAGTTGTCATGCAGCTATGCAGGACAATTCAG
AGTGATAGGACCAGGGTATCCCATCCGGGCTTTAGTTGGGGATGAAGCA
GAGCTGCCGTGCCGCATCTCTCCTGGGAAAAATGCCACGGGCATGGAGG
TGGGTTGGTACCGTTCTCCCTTCTCAAGAGTGGTTCACCTCTACCGAAA
TGGCAAGGACCAAGATGCAGAGCAAGCACCTGAATACCGGGGACGCACA
GAGCTTCTGAAAGAGACTATCAGTGAGGGAAAGGTTACCCTTAGGATTC
AGAACGTGAGATTCTCAGATGAAGGAGGCTACACCTGCTTCTTCAGAGA
CCACTCTTACCAAGAAGAGGCAGCAATGGAGTTGAAAGTGGAAGATCCC
TTCTATTGGGTCAACCCCGGTGTGCTGACTCTCATCGCACTTGTGCCTA
CGATCCTCCTGCAGGTCTCTGTAGGCCTTGTATTCCTCTTCCTGCAGCA
CAGACTGAGAGGAAAACTTCGTGCAGAAGTAGAGAATCTCCATCGGACT
TTTGATCCTCACTTCCTGAGGGTGCCCTGCTGGAAGATAACACTGTTTG
TTATTGTGCCTGTTCTTGGACCCCTGGTTGCCTTGATCCTGTGCTACAA
CTGGCTGCACCGAAGACTGGCAGGACAGTTTCTTGAAGAGCTAAGAAAC
CCCTTTTGA

Mus musculus full-length MOG protein sequence (with native leader, bold) (SEQ ID NO:5); mature MOG is underlined:

MACLWSFSWPSCFLSLLLLLLLQLSCSYAGQFRVIGPGYPIRALVGDEA
ELPCRISPGKNATGMEVGWYRSPFSRVVHLYRNGKDQDAEQAPEYRGRT
ELLKETISEGKVTLRIQNVRFSDEGGYTCFFRDHSYQEEAAMELKVEDP
FYWVNPGVLTLIALVPTILLQVSVGLVFLFLQHRLRGKLRAEVENLHRT
FDPHFLRVPCWKITLFVIVPVLGPLVALILCYNWLHRRLAGQFLEELRN
PF

Homo sapiens MOG: (with native leader, bold) (SEQ ID NO:6); mature MOG is underlined:

```
                10 20 30 40
MASLSRPSLP SCLCSFLLLL LLQVSSSYAG QFRVIGPRHP
                50 60 70 80
IRALVGDEVE LPCRISPGKN ATGMEVGWYR PPFSRVVHLY
                90 100 110 120
RNGKDQDGDQ APEYRGRTEL LKDAIGEGKV TLRIRNVRFS
              130 140 150 160
DEGGFTCFFR DHSYQEEAAM ELKVEDPFYW VSPGVLVLLA
              170 180 190 200
VLPVLLLQIT VGLIFLCLQY RLRGKLRAEI ENLHRTFDPH
              210 220 230 240
FLRVPCWKIT LFVIVPVLGP LVALIICYNW LHRRLAGQFL
EELRNPF
```

Macaca mulatta MOG: see, for example, NCBI Reference Sequence: NP_001181792.2 (SEQ ID NO:7)

| | |
|---|---|
| 1 | maslsrpslp sclcsfllll llqvsssyag qfrvigprqp iralvgdeve lpcrispgkn |
| 61 | atgmevgwyr ppfsrvvhly rngrdqdgeq apeyrgrtel lkdaigegkv tlrirnvrfs |
| 121 | deggftcffr dhsyqeeaai elkvedpfyw vspavlvlla vlpvlllqit vglvflclqy |
| 181 | rlrgklraei enlhrtfdph flrvpcwkit lfvivpvlgp lvaliicynw lhrrlagqfl |
| 241 | eelrnpf |

Callithrix jacchus MOG: see, for example, NCBI Reference Sequence: NP_001244171.1 (SEQ ID NO:8)

| | |
|---|---|
| 1 | maslskpslp sylcflllll hvsssyggqf rvigpshpiq alvgdaaelp crispgknat |
| 61 | gmevgwyrsp fsrvvhlyrn gkdqdgeqap eyrgrtellk ddigegkvtl kirnvrfpde |
| 121 | ggftcffrdh syqeeaamql kvedpfywvs pgvlvllavl pvlflqitvg lvflylqhrl |
| 181 | rgklraeien lhrtfdphfl rvpcwkitlf vivpvlgplv aliicynwlh rrlagqflee |
| 241 | lrnpf |

Study Sequences:

Mini-MOG (mMOG) sequences refer to the extracellular IgV-like domain of the full-length MOG protein.

Native leader+Mus musculus mini-MOG cDNA (SEQ ID NO:9):

ATGGCCTGTTTGTGGAGCTTCTCTTGGCCCAGCTGCTTCCTCTCCCTTC
TCCTCCTCCTTCTCCTCCAGTTGTCATGCAGCTATGCAGGACAATTCAG
AGTGATAGGACCAGGGTATCCCATCCGGGCTTTAGTTGGGGATGAAGCA
GAGCTGCCGTGCCGCATCTCTCCTGGGAAAAATGCCACGGGCATGGAGG
TGGGTTGGTACCGTTCTCCCTTCTCAAGAGTGGTTCACCTCTACCGAAA
TGGCAAGGACCAAGATGCAGAGCAAGCACCTGAATACCGGGGACGCACA
GAGCTTCTGAAAGAGACTATCAGTGAGGGAAAGGTTACCCTTAGGATTC
AGAACGTGAGATTCTCAGATGAAGGAGGCTACACCTGCTTCTTCAGAGA
CCACTCTTACCAAGAAGAGGCAGCAATGGAGTTGAAAGTGGAAGATCCC
TTCTATTGGGTCAACCCCGGTGTGCTGACTTGA

human chymotrypsinogen B2 signal peptide+Mus musculus mini-MOG cDNA (SEQ ID NO:10):

ATGGCCTTTCTGTGGCTGCTGTCCTGCTGGGCCCTGCTGGGGACCACCT
TTGGCGGACAATTCAGAGTGATAGGACCAGGGTATCCCATCCGGGCTTT
AGTTGGGGATGAAGCAGAGCTGCCGTGCCGCATCTCTCCTGGGAAAAAT
GCCACGGGCATGGAGGTGGGTTGGTACCGTTCTCCCTTCTCAAGAGTGG
TTCACCTCTACCGAAATGGCAAGGACCAAGATGCAGAGCAAGCACCTGA
ATACCGGGGACGCACAGAGCTTCTGAAAGAGACTATCAGTGAGGGAAAG
GTTACCCTTAGGATTCAGAACGTGAGATTCTCAGATGAAGGAGGCTACA
CCTGCTTCTTCAGAGACCACTCTTACCAAGAAGAGGCAGCAATGGAGTT
GAAAGTGGAAGATCCCTTCTATTGGGTCAACCCCGGTGTGCTGACTTGA
AGATC

human chymotrypsinogen B2 signal peptide+Mus musculus mini-MOG cDNA (start to stop) (SEQ ID NO:462):

ATGGCCTTTCTGTGGCTGCTGTCCTGCTGGGCCCTGCTGGGGACCACCT
TTGGCGGACAATTCAGAGTGATAGGACCAGGGTATCCCATCCGGGCTTT
AGTTGGGGATGAAGCAGAGCTGCCGTGCCGCATCTCTCCTGGGAAAAAT
GCCACGGGCATGGAGGTGGGTTGGTACCGTTCTCCCTTCTCAAGAGTGG
TTCACCTCTACCGAAATGGCAAGGACCAAGATGCAGAGCAAGCACCTGA
ATACCGGGGACGCACAGAGCTTCTGAAAGAGACTATCAGTGAGGGAAAG
GTTACCCTTAGGATTCAGAACGTGAGATTCTCAGATGAAGGAGGCTACA
CCTGCTTCTTCAGAGACCACTCTTACCAAGAAGAGGCAGCAATGGAGTT
GAAAGTGGAAGATCCCTTCTATTGGGTCAACCCCGGTGTGCTGACTTGA

HC7 leader+Mus musculus mini-MOG cDNA (SEQ ID NO:11):

ATGGAGTTTGGGCTGAGCTGGGTTTTCCTCGTTGCTCTTTTTAGAGGTG
TCCAGTGTGGACAATTCAGAGTGATAGGACCAGGGTATCCCATCCGGGC
TTTAGTTGGGGATGAAGCAGAGCTGCCGTGCCGCATCTCTCCTGGGAAA
AATGCCACGGGCATGGAGGTGGGTTGGTACCGTTCTCCCTTCTCAAGAG
TGGTTCACCTCTACCGAAATGGCAAGGACCAAGATGCAGAGCAAGCACC
TGAATACCGGGGACGCACAGAGCTTCTGAAAGAGACTATCAGTGAGGGA
AAGGTTACCCTTAGGATTCAGAACGTGAGATTCTCAGATGAAGGAGGCT
ACACCTGCTTCTTCAGAGACCACTCTTACCAAGAAGAGGCAGCAATGGA
GTTGAAAGTGGAAGATCCCTTCTATTGGGTCAACCCCGGTGTGCTGACT
TGA

Gaussia leader+Mus musculus mini-MOG cDNA (SEQ ID NO:12):

ATGGGAGTCAAAGTTCTGTTTGCCCTGATCTGCATCGCTGTGGCCGAGG
CCAAGCCCGGACAATTCAGAGTGATAGGACCAGGGTATCCCATCCGGGC
TTTAGTTGGGGATGAAGCAGAGCTGCCGTGCCGCATCTCTCCTGGGAAA
AATGCCACGGGCATGGAGGTGGGTTGGTACCGTTCTCCCTTCTCAAGAG
TGGTTCACCTCTACCGAAATGGCAAGGACCAAGATGCAGAGCAAGCACC
TGAATACCGGGGACGCACAGAGCTTCTGAAAGAGACTATCAGTGAGGGA
AAGGTTACCCTTAGGATTCAGAACGTGAGATTCTCAGATGAAGGAGGCT
ACACCTGCTTCTTCAGAGACCACTCTTACCAAGAAGAGGCAGCAATGGA
GTTGAAAGTGGAAGATCCCTTCTATTGGGTCAACCCCGGTGTGCTGACT
TGA

MOG Sequences

TM1-miniMOG (aa29-178) (SEQ ID NO:463)

GQFRVIGPGYPIRALVGDEAELPCRISPGKNATGMEVGWYRSPFSRVVH
LYRNGKDQDAEQAPEYRGRTELLKETISEGKVTLRIQNVRFSDEGGYTC
FFRDHSYQEEAAMELKVEDPFYWVNPGVLTLIALVPTILLQVPVGLVFL
FLT

TM0.6-miniMOG (aa29-169) (SEQ ID NO:464)

GQFRVIGPGYPIRALVGDEAELPCRISPGKNATGMEVGWYRSPFSRVVH
LYRNGKDQDAEQAPEYRGRTELLKETISEGKVTLRIQNVRFSDEGGYTC
FFRDHSYQEEAAMELKVEDPFYWVNPGVLTLIALVPTILLQVP

TM0.3-miniMOG (aa29-162) (SEQ ID NO:465)

GQFRVIGPGYPIRALVGDEAELPCRISPGKNATGMEVGWYRSPFSRVVH
LYRNGKDQDAEQAPEYRGRTELLKETISEGKVTLRIQNVRFSDEGGYTC
FFRDHSYQEEAAMELKVEDPFYWVNPGVLTLIALVP

Mus musculus MOG 1-117 (SEQ ID NO:466)

GQFRVIGPGYPIRALVGDEAELPCRISPGKNATGMEVGWYRSPFSRVVH
LYRNGKDQDAEQAPEYRGRTELLKETISEGKVTLRIQNVRFSDEGGYTC
FFRDHSYQEEAAMELKVED

Homo sapiens MOG 1-117 (SEQ ID NO:467)

GQFRVIGPRHPIRALVGDEVELPCRISPGKNATGMEVGWYRPPFSRVVH
LYRNGKDQDGDQAPEYRGRTELLKDAIGEGKVTLRIRNVRFSDEGGFTC
FFRDHSYQEEAAMELKVED

Macaca mulatta MOG 1-117 (SEQ ID NO:468)

GQFRVIGPRQPIRALVGDEVELPCRISPGKNATGMEVGWYRPPFSRVVH
LYRNGRDQDGEQAPEYRGRTELLKDAIGEGKVTIRIRNVRFSDEGGFTC
FFRDHSYQEEAAIELKVED

Callithrix jacchus MOG 1-117 (SEQ ID NO:469)

GQFRVIGPSHPIQALVGDAAELPCRISPGKNATGMEVGWYRSPFSRVVH
LYRNGKDQDGEQAPEYRGRTELLKDDIGEGKVTIKIRNVRFPDEGGFTC
FFRDHSYQEEAAMQLKVED

human chymotrypsinogen B2 signal peptide+Mus musculus MOG 1-117 (SEQ ID NO:470)

MAFLWLLSCWALLGTTFGGQFRVIGPGYPIRALVGDEAELPCRISPGKN
ATGMEVGWYRSPFSRVVHLYRNGKDQDAEQAPEYRGRTELLKETISEGK
VTLRIQNVRFSDEGGYTCFFRDHSYQEEAAMELKVED

human chymotrypsinogen B2 signal peptide+Homo sapiens MOG 1-117 (SEQ ID NO:471)

MAFLWLLSCWALLGTTFGGQFRVIGPRHPIRALVGDEVELPCRISPGKN
ATGMEVGWYRPPFSRVVHLYRNGKDQDGDQAPEYRGRTELLKDAIGEGK
VTLRIRNVRFSDEGGFTCFFRDHSYQEEAAMELKVED

human chymotrypsinogen B2 signal peptide+Macaca mulatta MOG 1-117 (SEQ ID NO:472)

MAFLWLLSCWALLGTTFGGQFRVIGPRQPIRALVGDEVELPCRISPGKN
ATGMEVGWYRPPFSRVVHLYRNGRDQDGEQAPEYRGRTELLKDAIGEGK
VTIRIRNVRFSDEGGFTCFFRDHSYQEEAAIELKVED

human chymotrypsinogen B2 signal peptide+Callithrix jacchus MOG 1-117 (SEQ ID NO:473)

MAFLWLLSCWALLGTTFGGQFRVIGPSHPIQALVGDAAELPCRISPGKN
ATGMEVGWYRSPFSRVVHLYRNGKDQDGEQAPEYRGRTELLKDDIGEGK
VTIKIRNVRFPDEGGFTCFFRDHSYQEEAAMQLKVED

TM1-miniMOG (aa29-178) (SEQ ID NO:474)

GGACAATTCAGAGTGATAGGACCAGGGTATCCCATCCGGGCTTTAGTTG
GGGATGAAGCAGAGCTGCCGTGCCGCATCTCTCCTGGGAAAAATGCCAC
GGGCATGGAGGTGGGTTGGTACCGTTCTCCCTTCTCAAGAGTGGTTCAC
CTCTACCGAAATGGCAAGGACCAAGATGCAGAGCAAGCACCTGAATACC
GGGGACGCACAGAGCTTCTGAAAGAGACTATCAGTGAGGGAAAGGTTAC
CCTTAGGATTCAGAACGTGAGATTCTCAGATGAAGGAGGCTACACCTGC
TTCTTCAGAGACCACTCTTACCAAGAAGAGGCAGCAATGGAGTTGAAAG
TGGAAGATCCCTTCTATTGGGTCAACCCCGGTGTGCTGACTCTCATCGC
ACTTGTGCCTACGATCCTCCTGCAGGTCTCTGTAGGCCTTGTATTCCTC
TTCCTG

TM0.6-miniMOG (aa29-169) (SEQ ID NO:475)

GGACAATTCAGAGTGATAGGACCAGGGTATCCCATCCGGGCTTTAGTTG
GGGATGAAGCAGAGCTGCCGTGCCGCATCTCTCCTGGGAAAAATGCCAC
GGGCATGGAGGTGGGTTGGTACCGTTCTCCCTTCTCAAGAGTGGTTCAC
CTCTACCGAAATGGCAAGGACCAAGATGCAGAGCAAGCACCTGAATACC
GGGGACGCACAGAGCTTCTGAAAGAGACTATCAGTGAGGGAAAGGTTAC
CCTTAGGATTCAGAACGTGAGATTCTCAGATGAAGGAGGCTACACCTGC
TTCTTCAGAGACCACTCTTACCAAGAAGAGGCAGCAATGGAGTTGAAAG
TGGAAGATCCCTTCTATTGGGTCAACCCCGGTGTGCTGACTCTCATCGC
ACTTGTGCCTACGATCCTCCTGCAGGTCTCT

TM0.3-miniMOG (aa29-162) (SEQ ID NO:476)

GGACAATTCAGAGTGATAGGACCAGGGTATCCCATCCGGGCTTTAGTTG
GGGATGAAGCAGAGCTGCCGTGCCGCATCTCTCCTGGGAAAAATGCCAC
GGGCATGGAGGTGGGTTGGTACCGTTCTCCCTTCTCAAGAGTGGTTCAC
CTCTACCGAAATGGCAAGGACCAAGATGCAGAGCAAGCACCTGAATACC
GGGGACGCACAGAGCTTCTGAAAGAGACTATCAGTGAGGGAAAGGTTAC
CCTTAGGATTCAGAACGTGAGATTCTCAGATGAAGGAGGCTACACCTGC
TTCTTCAGAGACCACTCTTACCAAGAAGAGGCAGCAATGGAGTTGAAAG
TGGAAGATCCCTTCTATTGGGTCAACCCCGGTGTGCTGACTCTCATCGC
ACTTGTGCCT

PLP Sequences

PLP1 (aa37-63) (SEQ ID NO:477)

HEALTGTEKLIETYFSKNYQDYEYLIN

PLP1+TM1 (aa10-63) (SEQ ID NO:478)

CLVGAPFASLVATGLCFFGVALFCGCGHEALTGTEKLIETYFSKNYQDY
EYLIN

PLP2 (aa89-151) (SEQ ID NO:479)

EGFYTTGAVRQIFGDYKTTICGKGLSATVTGGQKGRGSRGQHQAHSLER
VCHCLGKWLGHPDK

PLP2+TM2 (aa64-151) (SEQ ID NO:480)

VIHAFQYVIYGTASFFFLYGALLLAEGFYTTGAVRQIFGDYKTTICGKG
LSATVTGGQKGRGSRGQHQAHSLERVCHCLGKWLGHPDK

PLP3 (aa178-233) (SEQ ID NO:481)

FNTWTTCQSIAFPSKTSASIGSLCADARMYGVLPWNAFPGKVCGSNLLS
ICKTAEF

PLP3+TM3 (aa152-233) (SEQ ID NO:482)

FVGITYALTVVWLLVFACSAVPVYIYENTWTTCQSIAFPSKTSASIGSL
CADARMYGVLPWNAEPGKVCGSNLLSICKTAEF

PLP4 (aa261-277) (SEQ ID NO:483)

ATYNFAVLKLMGRGTKE

PLP4+TM4 (aa234-277) (SEQ ID NO:484)

QMTFHLFIAAFVGAAATLVSLLTFMIAATYNFAVLKLMGRGTKF

PLP1 (SEQ ID NO:485)

CACGAGGCCCTGACCGGCACCGAGAAGCTGATCGAGACCTACTTCAGCA
AGAACTACCAGGACTACGAGTACCTGATCAAC

PLP1+TM1 (SEQ ID NO:486)

TGCCTGGTGGGCGCCCCCTTCGCCAGCCTGGTGGCCACCGGCCTGTGCT
TCTTCGGCGTGGCCCTGTTCTGCGGCTGCGGCCACGAGGCCCTGACCGG
CACCGAGAAGCTGATCGAGACCTACTTCAGCAAGAACTACCAGGACTAC
GAGTACCTGATCAAC

PLP2 (SEQ ID NO:487)

GAGGGCTTCTACACCACCGGCGCCGTGAGGCAGATCTTCGGCGACTACA
AGACCACCATCTGCGGCAAGGGCCTGAGCGCCACCGTGACCGGCGGCCA
GAAGGGCAGGGGCAGCAGGGGCCAGCACCAGGCCCACAGCCTGGAGAGG
GTGTGCCACTGCCTGGGCAAGTGGCTGGGCCACCCCGACAAG

PLP2+TM2 (SEQ ID NO:488)

GTGATCCACGCCTTCCAGTACGTGATCTACGGCACCGCCAGCTTCTTCT
TCCTGTACGGCGCCCTGCTGCTGGCCGAGGGCTTCTACACCACCGGCGC
CGTGAGGCAGATCTTCGGCGACTACAAGACCACCATCTGCGGCAAGGGC
CTGAGCGCCACCGTGACCGGCGGCCAGAAGGGCAGGGGCAGCAGGGGCC
AGCACCAGGCCCACAGCCTGGAGAGGGTGTGCCACTGCCTGGGCAAGTG
GCTGGGCCACCCCGACAAG

PLP3 (SEQ ID NO:489)

TTCAACACCTGGACCACCTGCCAGAGCATCGCCTTCCCCAGCAAGACCA
GCGCCAGCATCGGCAGCCTGTGCGCCGACGCCAGGATGTACGGCGTGCT
GCCCTGGAACGCCTTCCCCGGCAAGGTGTGCGGCAGCAACCTGCTGAGC
ATCTGCAAGACCGCCGAGTTC

PLP3+TM3 (SEQ ID NO:490)

GGCATCACCTACGCCCTGACCGTGGTGTGGCTGCTGGTGTTCGCCTGCA
GCGCCGTGCCCGTGTACATCTACTTCAACACCTGGACCACCTGCCAGAG
CATCGCCTTCCCCAGCAAGACCAGCGCCAGCATCGGCAGCCTGTGCGCC
GACGCCAGGATGTACGGCGTGCTGCCCTGGAACGCCTTCCCCGGCAAGG
TGTGCGGCAGCAACCTGCTGAGCATCTGCAAGACCGCCGAGTTC

PLP4 (SEQ ID NO:491)

GCCACCTACAACTTCGCCGTGCTGAAGCTGATGGGCAGGGGCACCAAGT
TC

PLP4+TM4 (SEQ ID NO:492)

CAGATGACCTTCCACCTGTTCATCGCCGCCTTCGTGGGCGCCGCCGCCA
CCCTGGTGAGCCTGCTGACCTTCATGATCGCCGCCACCTACAACTTCGC
CGTGCTGAAGCTGATGGGCAGGGGCACCAAGTTC

TABLE 3
Exemplary leader sequences

| | | SEQ | | SEQ |
|---|---|---|---|---|
| ID | Amino Acid Sequence | ID NO | DNA sequence | ID NO |
| Native | MACLWSFSWPSCFLSLLLLL | 13 | ATGGCATGTTTATGGTCATTTTCATGGC | 26 |
| mouse | LLQLSCSYA | | CATCATGTTTTTTATCATTATTATTATT | |
| MOG | | | ATTATTATTACAATTATCATGTTCATAT | |
| | | | GCA | |
| HC1* | MELGLSWIFLLAILKGVQC | 14 | ATGGAGTTGGGACTGAGCTGGATTTTCC | 27 |
| | | | TTTTGGCTATTTTAAAAGGTGTCCAGTG | |
| | | | T | |
| HC2* | MELGLRWVFLVAILEGVQC | 15 | ATGGAACTGGGGCTCCGCTGGGTTTTCC | 28 |
| | | | TTGTTGCTATTTTAGAAGGTGTCCAGTG | |
| | | | T | |
| HC3* | MKHLWFFLLLVAAPRWVLS | 16 | ATGAAACACCTGTGGTTCTTCCTCCTGC | 29 |
| | | | TGGTGGCAGCTCCCAGATGGGTCCTGTC | |
| | | | C | |
| HC4* | MDWTWRILFLVAAATGAHS | 17 | ATGGACTGGACCTGGAGGATCCTCTTCT | 30 |
| | | | TGGTGGCAGCAGCAACAGGTGCCCACTC | |
| | | | G | |
| HC5″ | MDWTWRFLFVVAAATGVQS | 18 | ATGGACTGGACCTGGAGGTTCCTCTTTG | 31 |
| | | | TGGTGGCAGCAGCTACAGGTGTCCAGTC | |
| | | | C | |
| HC6 | MEFGLSWLFLVAILKGVQC | 19 | ATGGAGTTTGGGCTGAGCTGGCTTTTTC | 32 |
| | | | TTGTGGCGATTCTAAAAGGTGTCCAGTG | |
| | | | T | |
| HC7 | MEFGLSWVFLVALFRGVQC | 20 | ATGGAGTTTGGGCTGAGCTGGGTTTTCC | 33 |
| | | | TCGTTGCTCTTTTTAGAGGTGTCCAGTG | |
| | | | T | |
| HC8* | MDLLHKNMKHLWFFLLLVAA | 21 | ATGGACCTCCTGCACAAGAACATGAAAC | 34 |
| | PRWVLS | | ACCTGTGGTTCTTCCTCCTCCTGGTGGC | |
| | | | AGCTCCCAGATGGGTGCTGTCC | |
| LC1* | MDMRVPAQLLGLLLLWLSGA | 22 | ATGGACATGAGGGTCCCTGCTCAGCTCC | 35 |
| | RC | | TGGGGCTCCTGCTGCTCTGGCTCTCAGG | |
| | | | TGCCAGATGT | |
| LC2* | MKYLLPTAAAGLLLLAAQPA | 23 | ATGAAATACCTATTGCCTACGGCAGCCG | 36 |
| | MA | | CTGGATTGTTATTACTCGCGGCCCAGCC | |
| | | | GGCCATGGCC | |
| human | MAFLWLLSCWALLGTTFG | 24 | GCCTTTCTGTGGCTGCTGTCCTGCTGGG | 37 |
| chymotrypsinogen | | | CCCTGCTGGGGACCACCTTTGGC | |
| B2 signal | | | | |
| peptide | | | | |
| Gaussia | MGVKVLFALICIAVAEAKP | 25 | ATGGGAGTCAAAGTTCTGTTTGCCCTGA | 38 |
| | | | TCTGCATCGCTGTGGCCGAGGCCAAGCC | |
| | | | c | |

*Haryadi et al., 2015, PLoS ONE 10(2): e0116878

### Example 2

Exemplary Mus musculus proteolipid protein 1 (PLP) protein (SEQ ID NO:39):

Met Gly Leu Leu Glu Cys Cys Ala Arg Cys Leu Val
Gly Ala Pro Phe Ala Ser Leu Val Ala Thr Gly Leu
Cys Phe Phe Gly Val Ala Leu Phe Cys Gly Cys Gly
His Glu Ala Leu Thr Gly Thr Glu Lys Leu Ile Glu
Thr Tyr Phe Ser Lys Asn Tyr Gln Asp Tyr Glu Tyr
Leu Ile Asn Val Ile His Ala Phe Gln Tyr Val Ile
Tyr Gly Thr Ala Ser Phe Phe Phe Leu Tyr Gly Ala
Leu Leu Leu Ala Glu Gly Phe Tyr Thr Thr Gly Ala
Val Arg Gln Ile Phe Gly Asp Tyr Lys Thr Thr Ile
Cys Gly Lys Gly Leu Ser Ala Thr Val Thr Gly Gly
Gln Lys Gly Arg Gly Ser Arg Gly Gln His Gln Ala
His Ser Leu Glu Arg Val Cys His Cys Leu Gly Lys
Trp Leu Gly His Pro Asp Lys Phe Val Gly Ile Thr
Tyr Ala Leu Thr Val Val Trp Leu Leu Val Phe Ala
Cys Ser Ala Val Pro Val Tyr Ile Tyr Phe Asn Thr
Trp Thr Thr Cys Gln Ser Ile Ala Phe Pro Ser Lys
Thr Ser Ala Ser Ile Gly Ser Leu Cys Ala Asp Ala
Arg Met Tyr Gly Val Leu Pro Trp Asn Ala Phe Pro
Gly Lys Val Cys Gly Ser Asn Leu Leu Ser Ile Cys
Lys Thr Ala Glu Phe Gln Met Thr Phe His Leu Phe
Ile Ala Ala Phe Val Gly Ala Ala Ala Thr Leu Val
Ser Leu Leu Thr Phe Met Ile Ala Ala Thr Tyr Asn
Phe Ala Val Leu Lys Leu Met Gly Arg Gly Thr Lys
Phe

Exemplary Mus musculus myelin basic protein (MBP) protein (SEQ ID NO:40):

Met Gly Asn His Ser Gly Lys Arg Glu Leu Ser Ala
Glu Lys Ala Ser Lys Asp Gly Glu Ile His Arg Gly
Glu Ala Gly Lys Lys Arg Ser Val Gly Lys Leu Ser
Gln Thr Ala Ser Glu Asp Ser Asp Val Phe Gly Glu
Ala Asp Ala Ile Gln Asn Asn Gly Thr Ser Ala Glu
Asp Thr Ala Val Thr Asp Ser Lys His Thr Ala Asp
Pro Lys Asn Asn Trp Gln Gly Ala His Pro Ala Asp
Pro Gly Asn Arg Pro His Leu Ile Arg Leu Phe Ser
Arg Asp Ala Pro Gly Arg Glu Asp Asn Thr Phe Lys
Asp Arg Pro Ser Glu Ser Asp Glu Leu Gln Thr Ile
Gln Glu Asp Pro Thr Ala Ala Ser Gly Gly Leu Asp
Val Met Ala Ser Gln Lys Arg Pro Ser Gln Arg Ser
Lys Tyr Leu Ala Thr Ala Ser Thr Met Asp His Ala
Arg His Gly Phe Leu Pro Arg His Arg Asp Thr Gly
Ile Leu Asp Ser Ile Gly Arg Phe Phe Ser Gly Asp
Arg Gly Ala Pro Lys Arg Gly Ser Gly Lys Val Ser
Ser Glu Pro

Exemplary Homo sapiens myelin basic protein (MBP), transcript variant 7, protein (SEQ ID NO:41):

Met Gly Asn His Ala Gly Lys Arg Glu Leu Asn Ala
Glu Lys Ala Ser Thr Asn Ser Glu Thr Asn Arg Gly
Glu Ser Glu Lys Lys Arg Asn Leu Gly Glu Leu Ser
Arg Thr Thr Ser Glu Asp Asn Glu Val Phe Gly Glu
Ala Asp Ala Asn Gln Asn Asn Gly Thr Ser Ser Gln
Asp Thr Ala Val Thr Asp Ser Lys Arg Thr Ala Asp
Pro Lys Asn Ala Trp Gln Asp Ala His Pro Ala Asp
Pro Gly Ser Arg Pro His Leu Ile Arg Leu Phe Ser
Arg Asp Ala Pro Gly Arg Glu Asp Asn Thr Phe Lys
Asp Arg Pro Ser Glu Ser Asp Glu Leu Gln Thr Ile
Gln Glu Asp Ser Ala Ala Thr Ser Glu Ser Leu Asp
Val Met Ala Ser Gln Lys Arg Pro Ser Gln Arg His
Gly Ser Lys Tyr Leu Ala Thr Ala Ser Thr Met Asp
His Ala Arg His Gly Phe Leu Pro Arg His Arg Asp
Thr Gly Ile Leu Asp Ser Ile Gly Arg Phe Phe Gly
Gly Asp Arg Gly Ala Pro Lys Arg Gly Ser Gly Lys
Asp Ser His His Pro Ala Arg Thr Ala His Tyr Gly
Ser Leu Pro Gln Lys Ser His Gly Arg Thr Gln Asp
Glu Asn Pro Val Val His Phe Phe Lys Asn Ile Val
Thr Pro Arg Thr Pro Pro Pro Ser Gln Gly Lys Gly
Arg Gly Leu Ser Leu Ser Arg Phe Ser Trp Gly Ala
Glu Gly Gln Arg Pro Gly Phe Gly Tyr Gly Gly Arg
Ala Ser Asp Tyr Lys Ser Ala His Lys Gly Phe Lys
Gly Val Asp Ala Gln Gly Thr Leu Ser Lys Ile Phe
Lys Leu Gly Gly Arg Asp Ser Arg Ser Gly Ser Pro
Met Ala Arg Arg

Exemplary Homo sapiens myelin basic protein (MBP), transcript variant 1, protein (SEQ ID NO:42):

Met Ala Ser Gln Lys Arg Pro Ser Gln Arg His Gly
Ser Lys Tyr Leu Ala Thr Ala Ser Thr Met Asp His
Ala Arg His Gly Phe Leu Pro Arg His Arg Asp Thr
Gly Ile Leu Asp Ser Ile Gly Arg Phe Phe Gly Gly
Asp Arg Gly Ala Pro Lys Arg Gly Ser Gly Lys Val
Pro Trp Leu Lys Pro Gly Arg Ser Pro Leu Pro Ser
His Ala Arg Ser Gln Pro Gly Leu Cys Asn Met Tyr
Lys Asp Ser His His Pro Ala Arg Thr Ala His Tyr
Gly Ser Leu Pro Gln Lys Ser His Gly Arg Thr Gln
Asp Glu Asn Pro Val Val His Phe Phe Lys Asn Ile
Val Thr Pro Arg Thr Pro Pro Pro Ser Gln Gly Lys
Gly Arg Gly Leu Ser Leu Ser Arg Phe Ser Trp Gly
Ala Glu Gly Gln Arg Pro Gly Phe Gly Tyr Gly Gly
Arg Ala Ser Asp Tyr Lys Ser Ala His Lys Gly Phe
Lys Gly Val Asp Ala Gln Gly Thr Leu Ser Lys Ile
Phe Lys Leu Gly Gly Arg Asp Ser Arg Ser Gly Ser
Pro Met Ala Arg Arg

Exemplary Homo sapiens myelin basic protein (MBP), transcript variant 2, protein (SEQ ID NO:43):

Met Ala Ser Gln Lys Arg Pro Ser Gln Arg His Gly
Ser Lys Tyr Leu Ala Thr Ala Ser Thr Met Asp His
Ala Arg His Gly Phe Leu Pro Arg His Arg Asp Thr
Gly Ile Leu Asp Ser Ile Gly Arg Phe Phe Gly Gly
Asp Arg Gly Ala Pro Lys Arg Gly Ser Gly Lys Val
Pro Trp Leu Lys Pro Gly Arg Ser Pro Leu Pro Ser
His Ala Arg Ser Gln Pro Gly Leu Cys Asn Met Tyr
Lys Asp Ser His His Pro Ala Arg Thr Ala His Tyr
Gly Ser Leu Pro Gln Lys Ser His Gly Arg Thr Gln
Asp Glu Asn Pro Val Val His Phe Phe Lys Asn Ile
Val Thr Pro Arg Thr Pro Pro Pro Ser Gln Gly Lys
Gly Ala Glu Gly Gln Arg Pro Gly Phe Gly Tyr Gly
Gly Arg Ala Ser Asp Tyr Lys Ser Ala His Lys Gly
Phe Lys Gly Val Asp Ala Gln Gly Thr Leu Ser Lys
Ile Phe Lys Leu Gly Gly Arg Asp Ser Arg Ser Gly
Ser Pro Met Ala Arg Arg

Exemplary Homo sapiens myelin basic protein (MBP), transcript variant 3, protein (SEQ ID NO:44):

Met Ala Ser Gln Lys Arg Pro Ser Gln Arg His Gly
Ser Lys Tyr Leu Ala Thr Ala Ser Thr Met Asp His
Ala Arg His Gly Phe Leu Pro Arg His Arg Asp Thr
Gly Ile Leu Asp Ser Ile Gly Arg Phe Phe Gly Gly
Asp Arg Gly Ala Pro Lys Arg Gly Ser Gly Lys Asp
Ser His His Pro Ala Arg Thr Ala His Tyr Gly Ser
Leu Pro Gln Lys Ser His Gly Arg Thr Gln Asp Glu
Asn Pro Val Val His Phe Phe Lys Asn Ile Val Thr
Pro Arg Thr Pro Pro Pro Ser Gln Gly Lys Gly Arg
Gly Leu Ser Leu Ser Arg Phe Ser Trp Gly Ala Glu
Gly Gln Arg Pro Gly Phe Gly Tyr Gly Gly Arg Ala
Ser Asp Tyr Lys Ser Ala His Lys Gly Phe Lys Gly
Val Asp Ala Gln Gly Thr Leu Ser Lys Ile Phe Lys
Leu Gly Gly Arg Asp Ser Arg Ser Gly Ser Pro Met
Ala Arg Arg

Exemplary Homo sapiens myelin basic protein (MBP), transcript variant 4, protein (SEQ ID NO:45):

Met Ala Ser Gln Lys Arg Pro Ser Gln Arg His Gly
Ser Lys Tyr Leu Ala Thr Ala Ser Thr Met Asp His
Ala Arg His Gly Phe Leu Pro Arg His Arg Asp Thr
Gly Ile Leu Asp Ser Ile Gly Arg Phe Phe Gly Gly
Asp Arg Gly Ala Pro Lys Arg Gly Ser Gly Lys Asp
Ser His His Pro Ala Arg Thr Ala His Tyr Gly Ser
Leu Pro Gln Lys Ser His Gly Arg Thr Gln Asp Glu
Asn Pro Val Val His Phe Phe Lys Asn Ile Val Thr
Pro Arg Thr Pro Pro Pro Ser Gln Gly Lys Gly Ala
Glu Gly Gln Arg Pro Gly Phe Gly Tyr Gly Gly Arg
Ala Ser Asp Tyr Lys Ser Ala His Lys Gly Phe Lys
Gly Val Asp Ala Gln Gly Thr Leu Ser Lys Ile Phe
Lys Leu Gly Gly Arg Asp Ser Arg Ser Gly Ser Pro
Met Ala Arg Arg

Exemplary Homo sapiens myelin basic protein (MBP), transcript variant 8, protein (SEQ ID NO:46):

Met Gly Asn His Ala Gly Lys Arg Glu Leu Asn Ala
Glu Lys Ala Ser Thr Asn Ser Glu Thr Asn Arg Gly
Glu Ser Glu Lys Lys Arg Asn Leu Gly Glu Leu Ser
Arg Thr Thr Ser Glu Asp Asn Glu Val Phe Gly Glu
Ala Asp Ala Asn Gln Asn Asn Gly Thr Ser Ser Gln
Asp Thr Ala Val Thr Asp Ser Lys Arg Thr Ala Asp
Pro Lys Asn Ala Trp Gln Asp Ala His Pro Ala Asp
Pro Gly Ser Arg Pro His Leu Ile Arg Leu Phe Ser
Arg Asp Ala Pro Gly Arg Glu Asp Asn Thr Phe Lys
Asp Arg Pro Ser Glu Ser Asp Glu Leu Gln Thr Ile
Gln Glu Asp Ser Ala Ala Thr Ser Glu Ser Leu Asp
Val Met Ala Ser Gln Lys Arg Pro Ser Gln Arg His
Gly Ser Lys Tyr Leu Ala Thr Ala Ser Thr Met Asp
His Ala Arg His Gly Phe Leu Pro Arg His Arg Asp
Thr Gly Ile Leu Asp Ser Ile Gly Arg Phe Phe Gly
Gly Asp Arg Gly Ala Pro Lys Arg Gly Ser Gly Lys
Val Ser Ser Glu Glu

Exemplary Homo sapiens proteolipid protein 1 (PLP1), transcript variant 1, protein (SEQ ID NO:47):

Met Gly Leu Leu Glu Cys Cys Ala Arg Cys Leu Val
Gly Ala Pro Phe Ala Ser Leu Val Ala Thr Gly Leu
Cys Phe Phe Gly Val Ala Leu Phe Cys Gly Cys Gly
His Glu Ala Leu Thr Gly Thr Glu Lys Leu Ile Glu
Thr Tyr Phe Ser Lys Asn Tyr Gln Asp Tyr Glu Tyr
Leu Ile Asn Val Ile His Ala Phe Gln Tyr Val Ile
Tyr Gly Thr Ala Ser Phe Phe Phe Leu Tyr Gly Ala
Leu Leu Leu Ala Glu Gly Phe Tyr Thr Thr Gly Ala
Val Arg Gln Ile Phe Gly Asp Tyr Lys Thr Thr Ile
Cys Gly Lys Gly Leu Ser Ala Thr Val Thr Gly Gly
Gln Lys Gly Arg Gly Ser Arg Gly Gln His Gln Ala
His Ser Leu Glu Arg Val Cys His Cys Leu Gly Lys
Trp Leu Gly His Pro Asp Lys Phe Val Gly Ile Thr
Tyr Ala Leu Thr Val Val Trp Leu Leu Val Phe Ala
Cys Ser Ala Val Pro Val Tyr Ile Tyr Phe Asn Thr
Trp Thr Thr Cys Gln Ser Ile Ala Phe Pro Ser Lys
Thr Ser Ala Ser Ile Gly Ser Leu Cys Ala Asp Ala
Arg Met Tyr Gly Val Leu Pro Trp Asn Ala Phe Pro
Gly Lys Val Cys Gly Ser Asn Leu Leu Ser Ile Cys
Lys Thr Ala Glu Phe Gln Met Thr Phe His Leu Phe
Ile Ala Ala Phe Val Gly Ala Ala Ala Thr Leu Val
Ser Leu Leu Thr Phe Met Ile Ala Ala Thr Tyr Asn
Phe Ala Val Leu Lys Leu Met Gly Arg Gly Thr Lys
Phe

Exemplary Homo sapiens proteolipid protein 1 (PLP1), transcript variant 2, protein (SEQ ID NO:48):

Met Gly Leu Leu Glu Cys Cys Ala Arg Cys Leu Val
Gly Ala Pro Phe Ala Ser Leu Val Ala Thr Gly Leu
Cys Phe Phe Gly Val Ala Leu Phe Cys Gly Cys Gly
His Glu Ala Leu Thr Gly Thr Glu Lys Leu Ile Glu
Thr Tyr Phe Ser Lys Asn Tyr Gln Asp Tyr Glu Tyr
Leu Ile Asn Val Ile His Ala Phe Gln Tyr Val Ile
Tyr Gly Thr Ala Ser Phe Phe Phe Leu Tyr Gly Ala
Leu Leu Leu Ala Glu Gly Phe Tyr Thr Thr Gly Ala
Val Arg Gln Ile Phe Gly Asp Tyr Lys Thr Thr Ile
Cys Gly Lys Gly Leu Ser Ala Thr Phe Val Gly Ile
Thr Tyr Ala Leu Thr Val Val Trp Leu Leu Val Phe
Ala Cys Ser Ala Val Pro Val Tyr Ile Tyr Phe Asn
Thr Trp Thr Thr Cys Gln Ser Ile Ala Phe Pro Ser
Lys Thr Ser Ala Ser Ile Gly Ser Leu Cys Ala Asp
Ala Arg Met Tyr Gly Val Leu Pro Trp Asn Ala Phe
Pro Gly Lys Val Cys Gly Ser Asn Leu Leu Ser Ile
Cys Lys Thr Ala Glu Phe Gln Met Thr Phe His Leu
Phe Ile Ala Ala Phe Val Gly Ala Ala Ala Thr Leu
Val Ser Leu Leu Thr Phe Met Ile Ala Ala Thr Tyr
Asn Phe Ala Val Leu Lys Leu Met Gly Arg Gly Thr
Lys Phe

Exemplary Homo sapiens proteolipid protein 1 (PLP1), transcript variant 3, protein (SEQ ID NO:49):

Met Gly Leu Leu Glu Cys Cys Ala Arg Cys Leu Val
Gly Ala Pro Phe Ala Ser Leu Val Ala Thr Gly Leu
Cys Phe Phe Gly Val Ala Leu Phe Cys Gly Cys Gly
His Glu Ala Leu Thr Gly Thr Glu Lys Leu Ile Glu
Thr Tyr Phe Ser Lys Asn Tyr Gln Asp Tyr Glu Tyr
Leu Ile Asn Val Ile His Ala Phe Gln Tyr Val Ile
Tyr Gly Thr Ala Ser Phe Phe Phe Leu Tyr Gly Ala
Leu Leu Leu Ala Glu Gly Phe Tyr Thr Thr Gly Ala
Val Arg Gln Ile Phe Gly Asp Tyr Lys Thr Thr Ile
Cys Gly Lys Gly Leu Ser Ala Thr Val Thr Gly Gly
Gln Lys Gly Arg Gly Ser Arg Gly Gln His Gln Ala
His Ser Leu Glu Arg Val Cys His Cys Leu Gly Lys
Trp Leu Gly His Pro Asp Lys Phe Val Gly Ile Thr
Tyr Ala Leu Thr Val Val Trp Leu Leu Val Phe Ala
Cys Ser Ala Val Pro Val Tyr Ile Tyr Phe Asn Thr
Trp Thr Thr Cys Gln Ser Ile Ala Phe Pro Ser Lys
Thr Ser Ala Ser Ile Gly Ser Leu Cys Ala Asp Ala
Arg Met Tyr Gly Val Leu Pro Trp Asn Ala Phe Pro
Gly Lys Val Cys Gly Ser Asn Leu Leu Ser Ile Cys
Lys Thr Ala Glu Phe Gln Met Thr Phe His Leu Phe
Ile Ala Ala Phe Val Gly Ala Ala Ala Thr Leu Val
Ser Leu Leu Thr Phe Met Ile Ala Ala Thr Tyr Asn
Phe Ala Val Leu Lys Leu Met Gly Arg Gly Thr Lys
Phe

Exemplary Homo sapiens proteolipid protein 1 (PLP1), transcript variant 4, protein (SEQ ID NO:50):

Met Asp Tyr Glu Tyr Leu Ile Asn Val Ile His Ala
Phe Gln Tyr Val Ile Tyr Gly Thr Ala Ser Phe Phe
Phe Leu Tyr Gly Ala Leu Leu Leu Ala Glu Gly Phe
Tyr Thr Thr Gly Ala Val Arg Gln Ile Phe Gly Asp
Tyr Lys Thr Thr Ile Cys Gly Lys Gly Leu Ser Ala
Thr Val Thr Gly Gly Gln Lys Gly Arg Gly Ser Arg
Gly Gln His Gln Ala His Ser Leu Glu Arg Val Cys
His Cys Leu Gly Lys Trp Leu Gly His Pro Asp Lys
Phe Val Gly Ile Thr Tyr Ala Leu Thr Val Val Trp
Leu Leu Val Phe Ala Cys Ser Ala Val Pro Val Tyr
Ile Tyr Phe Asn Thr Trp Thr Thr Cys Gln Ser Ile
Ala Phe Pro Ser Lys Thr Ser Ala Ser Ile Gly Ser
Leu Cys Ala Asp Ala Arg Met Tyr Gly Val Leu Pro
Trp Asn Ala Phe Pro Gly Lys Val Cys Gly Ser Asn
Leu Leu Ser Ile Cys Lys Thr Ala Glu Phe Gln Met
Thr Phe His Leu Phe Ile Ala Ala Phe Val Gly Ala
Ala Ala Thr Leu Val Ser Leu Leu Thr Phe Met Ile
Ala Ala Thr Tyr Asn Phe Ala Val Leu Lys Leu Met
Gly Arg Gly Thr Lys Phe

### Example 3

In Vitro Expression Studies:

For each expression plasmid, Huh7 cells were grown to ˜80% confluency in wells of 12-well tissue culture dishes and transfected with 1 μg of plasmid, in duplicate, using Lipofectamine® (Invitrogen). Cell media were collected every 24 hr, and wells were replenished with fresh media for a total of three collections, or 72 hr. The media were spun at 4° C. for 10 minutes (min) at 10,000 rpms. The supernatants were collected, and the pelleted debris discarded. Phenylmethylsulfonyl fluoride (PMSF) was added to the final media samples. At the final collection (72 hr post-transfection) cell media were harvested and treated as described above. Cells were washed briefly with chilled phosphate-buffered saline. The wash was removed and chilled lysis buffer with protease inhibitors (Roche) was added to each well. Cells were incubated on ice for 30 min. The cell lysates were scraped from the dishes using a sterile cell-scraper, and suspensions collected. The cell lysates were incubated for 1 hr on ice with intermittent vortexing. The suspensions were spun at 4° C. for 10 min at 10,000 rpms. The supernatants were collected as the final cell lysates and the pellets were discarded. All samples were stored at −80° C. until further use.

Total Protein Quantification and Target Protein Analysis:

For total protein quantification, bicinchoninic acid (BCA) assays (Pierce) were performed on all samples in duplicate. Averages were taken as final concentrations.

Lysates and cell media samples were prepped to similar concentrations and volumes, and analyzed using the Wes™ automated western blotting system (ProteinSimple). Samples were heat denatured and loaded in a 2-40 kDa 24-well Wes plate (ProteinSimple), and probed with goat anti-mouse MOG antibody (Novus Biologics) at 1:100 and rabbit anti-GAPDH (glyceraldehyde 3-phosphate dehydrogenase) at 1:400. Anti-goat and anti-rabbit horseradish peroxidase (HRP) conjugates (ProteinSimple) were used for secondary detection. Standard Wes parameters were used, and samples were analyzed on Compass software (ProteinSimple).

Study Results:

To generate a portion of the MOG protein that could be secreted the extracellular domain (such as the the N-terminal 117 amino acids of the murine mature MOG protein (without the native signal peptide), i.e., amino acid residues 30-146 of SEQ ID NO:5, was fused to to various secretion sequences (Table 3). Expression plasmids were generated with each of the secretable mini-MOG (mMOG) sequences under the control of apolipoprotein E (ApoE) enhancer/human alpha 1-antitrypsin (hAAT) promoter (ApoE/hAAT) sequences. Huh7 cells, a liver cell line, were transduced with equal amounts of the expression plasmids. Cell lysates and timed media (supernatants) samples were analyzed for MOG protein expression.

Full-Length MOG is Retained in the Plasma Membrane of Lysed Huh7 Cells

To examine the secretability of the full-length MOG protein the plasma membrane and cell lysate and media of transformed Huh7 cells were examined for MOG expression. Huh7 cells were grown to ˜80% confluency and transformed using plasmids expressing full-length myelin oligodendrocyte glycoprotein (MOG) with various leader sequences: Wildtype (WT; native MOG leader), human chymotrypsinogen B2 signal peptide (“Sp7”; 18 amino acid signal peptide of NCBI reference sequence NP_001020371), or a combination of WT with the heavy chain 7 sequence (HC7s; Haryadi et al., PloS ONE 10(2): e0116878. doi:10.1371/journal.pone.0116878 (2015)). Cells and media were harvested 72 hr post-transfection. Plasma membrane fractions from cells were enriched as described (Nishiumi et al., Biosci. Biotechnol. Biochem. 71 (9), 2343-2346, (2007)) Briefly, cells were harvested in modified Buffer A (50 mM Tris, 0.5 mM DTT) with protease inhibitors (cOmplete™ tablets, Roche) and 0.1% Triton X100. The cells were sheared by passage through a 25-gauge needle; 3 times. The homogenate was centrifuged for 10 min at 1000×g at 4° C. The supernatant was harvested as post-plasma membrane (PPM) fraction. The pellet was resuspended in Buffer A without Triton X100 and incubated on ice for 10 min with occasional mixing and recentrifuged at 1000×g for 10 min at 4° C. The supernatant was harvested and added to the PPM fraction. The pellet was resuspended in Buffer A with 1% Triton X100 and incubated on ice for 1 hr with occasional mixing. The suspension was then centrifuged at 16000×g for 20 min at 4° C. The supernatant was harvested as the plasma membrane (PM) fraction. All fractions, including PM and the cell lysate (essentially the PPM fraction) and media were assayed for protein concentrations (BCA assay; Pierce) and equivalent sample concentrations were heat denatured and loaded in a 2-40 kDa 24-well WES plate (ProteinSimple). Samples were probed with goat anti-mouse MOG antibody at 1:100 and rabbit anti-GAPDH at 1:400. Anti-goat and anti-rabbit HRP conjugates (ProteinSimple) were used for secondary detection. Standard WES parameters were used, and samples were analyzed on Compass Software (ProteinSimple).

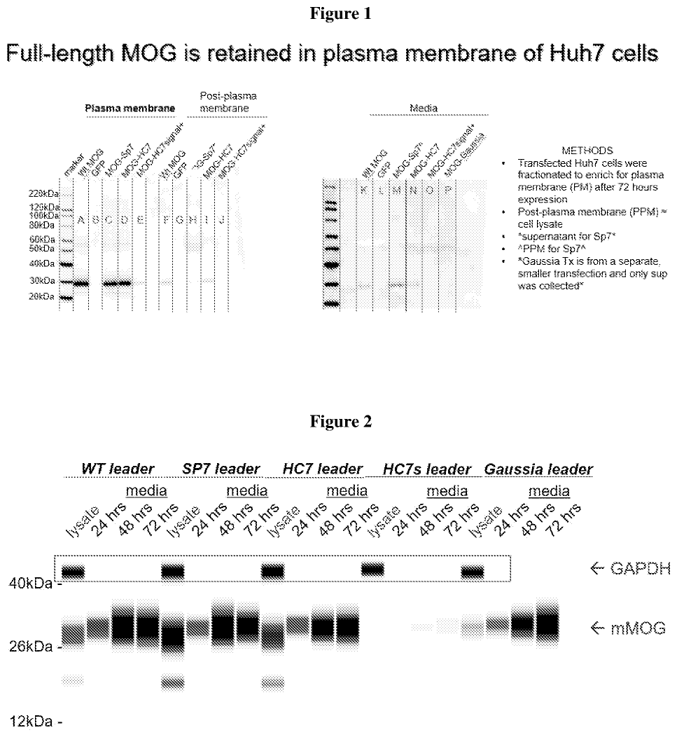

FIG. 1 shows that the full-length MOG, regardless of the leader signal used, is retained in the plasma membrane. Retention of MOG in the plasma membrane is due to the transmembrane and transmembrane associated domain.

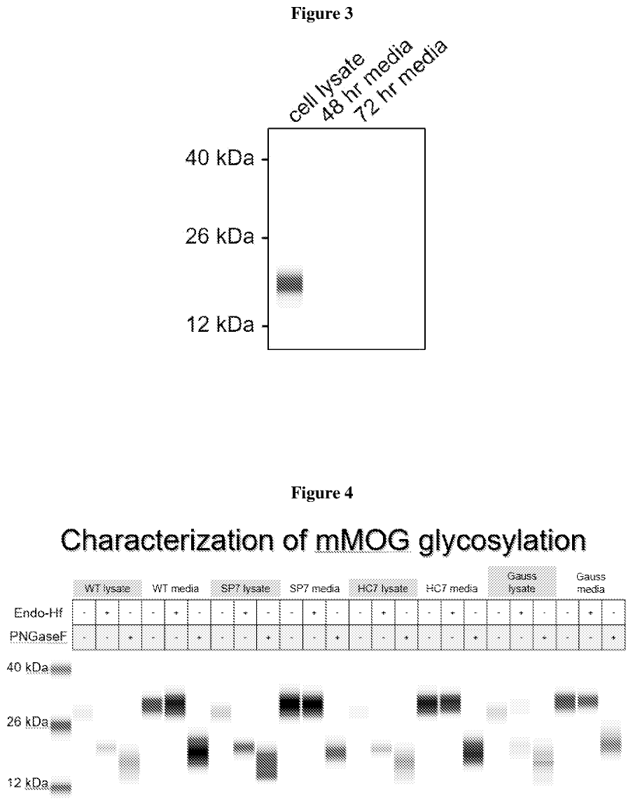

All leaders, except for control HC7s, allowed mMOG expression in the cell lysate and secretion of mMOG in the cell media at 24-, 48-, and 72-hr post-transfection (FIG. 2). A separate control plasmid was generated for expression of mini-MOG without any leader sequence. This MOG protein was present in the cell lysate, but not secreted or released into the cell media, as evidenced by the lack of detectable mMOG at 48- and 72-hr post-transfection (FIG. 3).

Characterization of MOG Glycosylation

To further characterize the secreted protein the glycosylation profile of the predicted N-linked glycosylation moiety reportedly at ASN60 was examined. Briefly, equivalent volumes of cell lysate and media were treated with Endo-Hf (NEB) or PNGaseF (NEB) according to the manufacturer's protocols. Samples were then assayed for size shifts due to deglycosylation using the WES system. Cell lysates were susceptible to both Endo H and PNGase F most likely due to various stages of glycosylation occurring during translation; however, secreted MOG in the media was only susceptible to PNGase F (FIG. 4). This indicates a complex N-linked glycan on the secreted protein.

Expression of mMOG in Huh7 Cells after AAV-Mediated Delivery

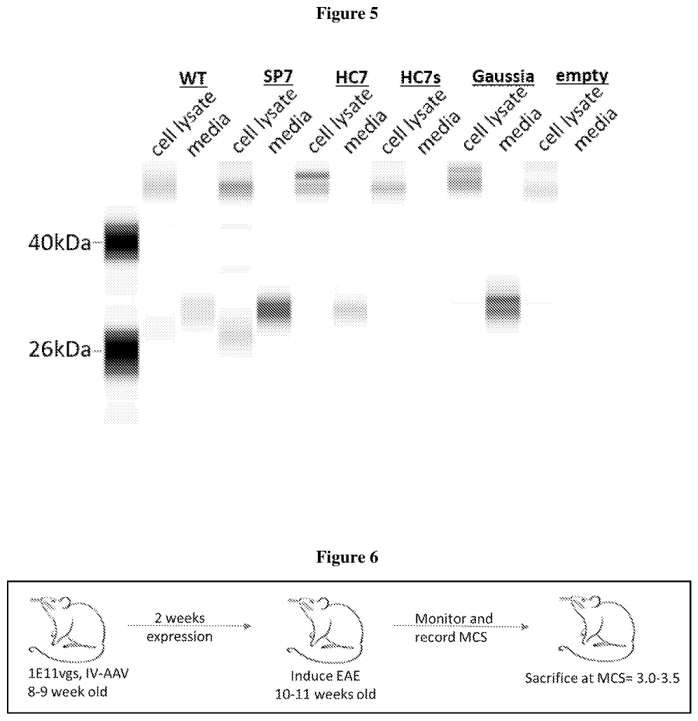

To analyze the expression profile of mMOG delivered by way of an AAV vector, crude AAV.mMOG was generated in HEK293 cells. Briefly, HEK293 cells were triple-transfected with helper plasmid, Spk2 (SEQ ID NO:2) trans-plasmid, and the mMOG cis-plasmids with the various secretory leader sequences (Table 3). Cell media was harvested 60 hr post-transfection and placed on confluent Huh7 cells. The Huh7 cells and media were harvested 72 hr post-transfection and assayed for mMOG expression. The results confirm that mMOG sequences were able to be packaged in Spk2, mMOG was expressed in Huh7 cells, and mMOG was secreted by the cells (FIG. 5).

### Example 4

Further exemplary unwanted antigens:

TABLE 4

| | | SEQ |
|---|---|---|
| | Sequence | ID NO: |
| Myelin Basic Protein | | |
| (MBP) Derived Peptides: | | |
| MBP 30-44 | PRHRDTGILDSIGRF | 51 |
| MBP 83-99 | ENPVVHFFKNIVTPRTP | 52 |
| MBP 131-145 | ASDYKSAHKGFKGVD | 53 |
| MBP 140-154 | GFKGVDAQGTLSKIF | 54 |
| Interphotoreceptor | | |
| Retinoid Binding | | |
| Protein (IRBP) Peptide: | | |
| IRBP 1177-1191 | ADGSSWEGVGVVPDV | 55 |

TABLE 5

| Thyroid Stimulating | | |
|---|---|---|
| Hormone Receptor | | SEQ |
| Derived Peptides | Sequence | ID NO: |
| RNB-4K-GKK | KKGNLPNISRIYVSIDVTGKK | 56 |
| RNB-5D-K1 | KKKKYVSIDVTLQQLESHKKK | 57 |
| RNB-9B | GLKMFPDLTKVYSTD | 58 |

TABLE 6

| S-Arrestin | | SEQ ID |
|---|---|---|
| Derived Peptides | Sequence | NO: |
| 1 | IFKKISRDKSVTIYL | 59 |
| 2 | KGKKVYVTLTCAFRY | 60 |
| 3 | VIGLTFRRDLYFSRVQVYPPVG | 61 |
| 4 | ESLLKKLGSNTYPFLLTFPDYLPCSVMLQPAPQ | 62 |
| | DSGK | |
| 5 | SSVRLLIRKVQHAPLEM | 63 |
| 6 | AEAAWQFFMSDKPLHLAVSLNKEIYF | 64 |
| 7 | LTKTLTLLPLLANNRERRGIALDGKIKHEDTNL | 65 |
| | ASSTIIKE | |
| 8 | IDRTVLGILVSYQIKVKLTVS | 66 |
| 9 | NTEKTVKKIKAFVEQVANVVLYSSDYYVK | 67 |
| HIP-115 | MAASGKTSKSEPNHVIFKKISRDKSVTIYLGNR | 68 |
| | DYIDHVSQV | |
| HIP-115NE | HVIFKKISRDKSVTIYLGN | 69 |
| HIP-115NE1 | KKISRDKSVTI | 70 |
| HIP-115NE2 | FKKISRDKSVTIY | 71 |
| HIP-115NE3 | VIFKKISRDKSVTIYLG | 72 |
| HIP-115NE1-KKK | KKKVIFKKISRDKSVTIYLGKKK | 73 |
| HIP-241125 | LTLLPLLANNRERRGIALDGKIKHEDTNLASSTI | 74 |
| | IKEG | |
| HIP-11AC | NRERRGIALDGKIKHED | 75 |
| HIP-11AC1 | ERRGIALDGKIKH | 76 |
| HIP-11B | RERRGIALDGKIKHE | 77 |
| HIP-11JO | DGKIKHEDTNLASSTIIKEG | 78 |
| HIP-11JO1 | HEDTNLASST | 79 |
| HIP-11JO2 | KHEDTNLASSTI | 80 |
| HIP-11J03 | IKHEDTNLASSTII | 81 |
| HIP-11JO4 | KIKHEDTNLASSTIIK | 82 |
| HIP-24DG | LTKTLTLLPLLANNRERR | 83 |
| HIP-24HM | LTLLPLLANNRERRGIALDG | 84 |
| HIP-24HM1 | LLANNRERRG | 85 |
| HIP-24HM2 | PLLANNRERRGI | 86 |
| HIP-24HM3 | LPLLANNRERRGIA | 87 |
| HIP-24P | NNRERRGIALDGKIK | 88 |
| HIP-9FL | VKKIKAFVEQVANVVLYSSDY | 89 |
| HIP-9FL-KKK | KKKVKKIKAFVEQVANVVLYSSDYKKK | 90 |
| HIP-9K1 | AFVEQVANVVL | 91 |
| HIP-9K1-KKK | KKKAFVEQVANVVLKKK | 92 |
| HIP-17GN | VIGLTFRRDLYFSRVQVYPPVG | 93 |
| HIP-17GN-KKK | KKKVIGLTFRRDLYFSRVQVYPPVGKKK | 94 |
| HIP-17J | LTFRRDLYFSRVQVY | 95 |
| HIP-17J-KKK | KKKLTFRRDLYFSRVQVYKKK | 96 |
| HIP-17K1 | TFRRDLYFSRVQ | 97 |
| HIP-17M1 | RRDLYFSRVQ | 98 |
| HIP-12AK | IDRTVLGILVSYQIKVKLTVSGFLG | 99 |
| HIP-12AK-KKK | KKKIDRTVLGILVSYQIKVKLTVSGFLGKKK | 100 |
| HIP-12E | VLGILVSYQIKVKLT | 101 |
| HIP-12E-KKK | KKKVLGILVSYQIKVKLTKKK | 102 |
| HIP-12G1 | GILVSYQIKVK | 103 |
| HIP-12G1-KKK | KKKGILVSYQIKVKKKK | 104 |

TABLE 7

| Myosin | | |
|---|---|---|
| Derived | | |
| Peptides | Sequence | SEQ ID NO: |
| 1 | MLTFLHEPAVLFNLK | 105 |
| 2 | VNPYKWLPVYNAEW | 106 |
| 3 | PHIFSISDNAYQYML | 107 |
| 4 | KRVIQYFASIAAIGD | 108 |
| 5 | YHIFYQILSNKKPEL | 109 |
| 6 | KSAYLMGLNSADLLK | 110 |
| 7 | KSSLKLMATLFSSYA | 111 |
| 8 | KGSSFQTVSALHREN | 112 |
| 9 | EATLQHEATAAALRK | 113 |
| 10 | RVQLLHSQNTSLINQ | 114 |

TABLE 8

| Thyroid Stimulating | | |
|---|---|---|
| Hormone Receptor | | |
| Derived Peptides | Sequence | SEQ ID NO: |
| RNB_5 (1) | ISRIYVSIDVTLQQLESHSFYNLSKVTHI | 115 |
| RNB 4 (2) | LRTIPSHAFSNLPNISRIYVSIDVTLQQL | 116 |
| RNB 4A | LRTIPSHAFSNLPNI | 117 |
| RNB 4B | RTIPSHAFSNLPNIS | 118 |
| RNB 4C | TIPSHAFSNLPNISR | 119 |
| RNB 4D | IPSHAFSNLPNISRI | 120 |
| RNB 4E | PSHAFSNLPNISRIY | 121 |
| RNB 4F | SHAFSNLPNISRIYV | 122 |
| RNB 4G | HAFSNLPNISRIYVS | 123 |
| RNB 4H | AFSNLPNISRIYVSI | 124 |
| RNB 4I | FSNLPNISRIYVSID | 125 |
| RNB 4J | SNLPNISRIYVSIDV | 126 |
| RNB 4K | NLPNISRIYVSIDVT | 127 |
| RNB 4L | LPNISRIYVSIDVTL | 128 |
| RNB 4M | PNISRIYVSIDVTLQ | 129 |
| RNB 4N | NISRIYVSIDVTLQQ | 130 |
| RNB 4J-GKK | KKGSNLPNISRIYVSIDVGKK | 131 |
| RNB 4K-GKK | KKGNLPNISRIYVSIDVTGKK | 132 |
| RNB_9 (3) | TGLKMFPDLTKVYSTDIFFILEITDNPYM | 133 |
| RNB 9A | TGLKMFPDLTKVYST | 134 |
| RNB 9B | GLKMFPDLTKVYSTD | 135 |
| RNB 9C | LKMFPDLTKVYSTDI | 136 |
| RNB 9D | KMFPDLTKVYSTDTF | 137 |
| RNB_5A (6) | ISRIYVSIDVTLQQL | 138 |
| RNB_5B (7) | SRIYVSIDVTLQQLE | 139 |
| RNB 5C (8) | RIYVSIDVTLQQLES | 140 |
| RNB 5D (9) | IYVSIDVTLQQLESH | 141 |
| RNB 5E (10) | YVSIDVTLQQLESHS | 142 |
| RNB 5F (11) | VSIDVTLQQLESHSF | 143 |
| RNB 5G | SIDVTLQQLESHSFY | 144 |
| RNB 5H | IDVTLQQLESHSFYN | 145 |
| RNB 5I | DVTLQQLESHSFYNL | 146 |
| RNB 5D-GKK (12) | KKGIYVSIDVTLQQLESHGKK | 147 |
| RNB_5E-GKK (13) | KKGYVSIDVTLQQLESHSGKK | 148 |
| RNB 5F-GKK (14) | KKGVSIDVTLQQLESHSFGKK | 149 |
| RNB 5J | VTLQQLESHSFYNLS | 150 |
| RNB 5K | TLQQLESHSFYNLSK | 151 |
| RNB 5L | LQQLESHSFYNLSKV | 152 |
| RNB 5M | QQLESHSFYNLSKVT | 153 |
| RNB 5N | QLESHSFYNLSKVTH | 154 |
| RNB 5O | LESHSFYNLSKVTHI | 155 |
| RNB_4J | SNLPNISRIYVSIDV | 156 |
| RNB 4J-GKK | KKGSNLPNISRIYVSIDVGKK | 157 |
| RNB 9A (17) | TGLKMFPDLTKVYST | 158 |
| RNB 9B (18) | GLKMFPDLTKVYSTD | 159 |
| RNB_9C (19) | LKMFPDLTKVYSTDI | 160 |
| RNB 9D (20) | KMFPDLTKVYSTDIF | 161 |
| (21) | KKKIYVSIDVTLQQLESHKKK | 162 |
| (22) | KKGKYVSIDVTLQQLESHGKK | 163 |
| (23) | KKGIKVSIDVTLQQLESHGKK | 164 |
| (24) | KKGIYKSIDVTLQQLESHGKK | 165 |
| (25) | KKGIYVSIDVKLQQLESHGKK | 166 |
| (26) | KKGIYVSIDVTLQKLESHGKK | 167 |
| (27) | KKGIYVSIDVTLQQKESHGKK | 168 |
| (28) | KKGIYVSIDVTLQQLKSHGKK | 169 |
| (29) | KKGIYVSIDVTLQQLEKHGKK | 170 |
| (30) | KKGIYVSIDVTLQQLESKGKK | 171 |
| (31) | KKGYVSIDVTLQQLEGKK | 172 |
| (32) | KKGYVSIDVKLQQLEGKK | 173 |
| (33) | KKGYVSIDVTLQKLEGKK | 174 |
| (34) | KKGYVSIDVTLQQKEGKK | 175 |
| (35) | KKGYVSIDVKLQKKEGKK | 176 |
| (36) | KKGIYVSIDVTLQQLEGKK | 177 |
| (37) | KKGIYVSIDVKLQQLEGKK | 178 |
| (38) | KKGIYVSIDVTLQKLEGKK | 179 |
| (39) | KKGIYVSIDVTLQQKEGKK | 180 |
| (40) | KKGIYVSIDVKLQKKEGKK | 181 |
| (41) | KKGTYVSIDVTLQQLEGKK | 182 |
| (42) | KKGTYVSIDVKLQQLEGKK | 183 |
| (43) | KKGTYVSIDVTLQKLEGKK | 184 |
| (44) | KKGTYVSIDVTLQQKEGKK | 185 |
| (45) | KKGTYVSIDVKLQKKEGKK | 186 |
| (46) | KKKKYVSIDVTLQQLESHKKK | 187 |
| (47) | KKKIKVSIDVTLQQLESHKKK | 188 |
| (48) | KKKIYKSIDVTLQQLESHKKK | 189 |
| (49) | KKKIYVKIDVTLQQLESHKKK | 190 |
| (50) | KKKIYVSIDVKLQQLESHKKK | 191 |
| (51) | KKKIYVSIDVTLKQLESHKKK | 192 |
| (52) | KKKIYVSIDVTLQKLESHKKK | 193 |
| (53) | KKKIYVSIDVTLQQKESHKKK | 194 |
| (54) | KKKIYVSIDVTLQQLKSHKKK | 195 |
| (55) | KKKIYVSIDVTLQQLEKHKKK | 196 |
| (56) | KKKIYVSIDVTLQQLESKKKK | 197 |
| (57) | KKKYVSIDVTLQQLEKKK | 198 |
| (58) | KKKYVSIDVKLQQLEKKK | 199 |
| (59) | KKKYVSIDVTLQKLEKKK | 200 |
| (60) | KKKYVSIDVTLQQKEKKK | 201 |
| (61) | KKKYVSIDVKLQKKEKKK | 202 |
| (62) | NLPNISRIYVSIDVT | 203 |
| (63) | KKGNLPNISRIYVSIDVTGKK | 204 |
| (64) | LTLKLYNNGFTSVQGYAFNGTKLDAVYL | 205 |
| (65) | LTLKLYNNGFTSVQGY | 206 |
| (66) | TLKLYNNGFTSVQGY | 207 |
| (67) | KKKTLKLYNNGFTSVQGYKKK | 208 |
| (68) | LKLYNNGFTSVQGYA | 209 |
| (69) | KLYNNGFTSVQGYAF | 210 |
| (70) | LYNNGFTSVQGYAFN | 211 |
| (71) | YNNGFTSVQGYAFNG | 212 |
| (72) | NNGFTSVQGYAFNGT | 213 |
| (73) | NGFTSVQGYAFNGTK | 214 |
| (74) | GFTSVQGYAFNGTKL | 215 |
| (75) | FTSVQGYAFNGTKLD | 216 |
| (76) | TSVQGYAFNGTKLDA | 217 |
| (77) | SVQGYAFNGTKLDAV | 218 |
| (78) | VQGYAFNGTKLDAVY | 219 |
| RNB 1 | GGMGCSSPPCECHQEEDFRVTCKDIQRIP | 220 |
| RNB 2 | EEDFRVTCKDIQRIPSLPPSTQTLKLIETH | 221 |
| RNB 3 | SLPPSTQTLKLIETHLRTIPSHAFSNLPNI | 222 |
| RNB 6 | ESHSFYNLSKVTHIEIRNTRNLTYIDPDA | 223 |
| RNB 7 | EIRNTRNLTYIDPDALKELPLLKFLGIFN | 224 |
| RNB 8 | LKELPLLKFLGIFNTGLKMFPDLTKVYST | 225 |
| RNB 9 | TGLKMFPDLTKVYSTDIFFILEITDNPYM | 226 |
| RNB 10 | TDIFFILEITDNPYMTSIPVNAFQGLCNET | 227 |
| RNB 11 | TSIPVNAFQGLCNETLTLKLYNNGFTSV | 228 |
| RNB 12 | LTLKLYNNGFTSVQGYAFNGTKLDAVYL | 229 |
| RNB 13 | QGYAFNGTKLDAVYLNKNKYLTVIDKDA | 230 |
| RNB 14 | NKNKYLTVIDKDAFGGVYSGPSLLDVSQ | 231 |
| RNB 15 | FGGVYSGPSLLDVSQTSVTALPSKGLEH | 232 |
| RNB 16 | TSVTALPSKGLEHLKELIARNTWTLKKL | 233 |
| RNB 17 | LKELIARNTWTLKKLPLSLSFLHLTRAD | 234 |
| RNB 18 | PLSLSFLHLTRADLSYPSHCCAFKNQKK | 235 |
| RNB 19 | LSYPSHCCAFKNQKKIRGILESLMCNES | 236 |
| RNB 20 | IRGILESLMCNESSMQSLRQRKSVNALN | 237 |
| RNB 21 | SMQSLRQRKSVNALNSPLHQEYEENLGDS | 238 |
| RNB 22 | SPLHQEYEENLGDSIVGYKEKSKFQDTHN | 239 |
| RNB 23 | IVGYKEKSKFQDTHNNAHYYVFFEEQEDE | 240 |
| RNB 24 | NAHYYVFFEEQEDEIIGFGQELKNPQEET | 241 |
| RNB 25 | IIGFGQELKNPQEETLQAFDSHYDYTICG | 242 |
| RNB 26 | LQAFDSHYDYTICGDSEDMVCTPKSDEFN | 243 |
| RNB 27 | DSEDMVCTPKSDEFNPCEDIMGYKFLR | 244 |
| RNB 28 | KLDAVYLNKNKYLTVIDKDAFGGVYSGPS | 245 |

TABLE 9

| Myelin | | |
|---|---|---|
| Oligodendrocyte | | SEQ |
| Glycoprotein (MOG) | | ID |
| Derived Peptides | Sequence | NO: |
| MOG 41-55 | RPPFSRVVHLYRNGK | 246 |
| MOG 43-57 | PFSRVVHLYRNGKDQ | 247 |
| MOG 44-58 | FSRVVHLYRNGKDQD | 248 |
| MOG 45-59 | SRVVHLYRNGKDQDG | 249 |
| MOG 35-55 | MEVGWYRSPFSRVVHLYRNGK | 250 |
| MOG 40-60 | YRPPFSRVVHLYRNGKDQDGD | 251 |

TABLE 10

| Proteolipid | | |
|---|---|---|
| Protein (PLP) | | SEQ |
| Derived | | ID |
| Peptides | Sequence | NO: |
| PLP 36-61 | HEALTGTEKLIETYFSKNYQDYEYLI | 252 |
| PLP 179-206 | TWTTCQSIAFPSKTSASIGSLCADARMY | 253 |
| PLP 207-234 | GVLPWNAFPGKVCGSNLLSICKTAEFQM | 254 |
| PLP 39-57 | LTGTEKLIETYFSKNYQDY | 255 |
| PLP 180-198 | WTTCQSIAFPSKTSASIGS | 256 |
| PLP 208-222 | VLPWNAFPGKVCGSN | 257 |
| PLP 39-53 | LTGTEKLIETYFSKN | 258 |
| PLP 42-56 | TEKLIETYFSKNYQD | 259 |
| PLP 43-57 | EKLIETYFSKNYQDY | 260 |
| PLP 180-194 | WTTCQSIAFPSKTSA | 261 |
| PLP 181-195 | TTCQSIAFPSKTSAS | 262 |
| PLP 182-196 | TCQSIAFPSKTSASI | 263 |
| PLP 183-197 | CQSIAFPSKTSASIG | 264 |
| PLP 184-198 | QSIAFPSKTSASIGS | 265 |
| PLP 208-222 | VLPWNAFPGKVCGSN | 266 |
| POP-1 | HEALTGTEKLIETYF | 267 |
| POP-2 | EALTGTEKLIETYFS | 268 |
| POP-3 | ALTGTEKLIETYFSK | 269 |
| POP-4 | LTGTEKLIETYFSKN | 270 |
| POP-5 | TGTEKLIETYFSKNY | 271 |
| POP-6 | GTEKLIETYFSKNYQ | 272 |
| POP-7 | TEKLIETYFSKNYQD | 273 |
| POP-8 | EKLIETYFSKNYQDY | 274 |
| POP-9 | KLIETYFSKNYQDYE | 275 |
| POP-10 | LIETYFSKNYQDYEY | 276 |
| POP-11 | IETYFSKNYQDYEYL | 277 |
| POP-12 | ETYFSKNYQDYEYLI | 278 |
| POP-14 | WTTCQSIAFPSKTSA | 279 |
| POP-15 | TCQSIAPPSKTSASI | 280 |
| POP-16 | CQSIAFPSKTSASIG | 281 |
| POP-18 | QSIAFPSKTSASIGS | 282 |

TABLE 11

| FVIII | | SEQ |
|---|---|---|
| Derived | | ID |
| Peptides | Sequence | NO: |
| 1 | KKGPRCLTRYYSSFVNMEGKK | 283 |
| 2 | KKGPRCLTRYYSSFVNMEGKE | 284 |
| 3 | KKGPRCLTRYYSSFVNMEGEK | 285 |
| 4 | KKGPRCLTRYYSSFVNMEGEE | 286 |
| 5 | EEGPRCLTRYYSSFVNMEGKK | 287 |
| 6 | EEGPRCLTRYYSSFVNMEGKE | 288 |
| 7 | EEGPRCLTRYYSSFVNMEGEK | 289 |
| 8 | EEGPRCLTRYYSSFVNMEGEE | 290 |
| 9 | KEGPRCLTRYYSSFVNMEGKE | 291 |
| 10 | KEGPRCLTRYYSSFVNMEGKE | 292 |
| 11 | KEGPRCLTRYYSSFVNMEGEK | 293 |
| 12 | KEGPRCLTRYYSSFVNMEGEE | 294 |
| 13 | EKGPRCLTRYYSSFVNMEGKK | 295 |
| 14 | EKGPRCLTRYYSSFVNMEGKE | 296 |
| 15 | EKGPRCLTRYYSSFVNMEGEK | 297 |
| 16 | EKGPRCLTRYYSSFVNMEGEE | 298 |
| 17 | KKGDNIMVTFRNQASRPYGKK | 299 |
| 18 | KKGDNIMVTFRNQASRPYGKE | 300 |
| 19 | KKGDNIMVTFRNQASRPYGEK | 301 |
| 20 | KKGDNIMVTFRNQASRPYGEE | 302 |
| 21 | EEGDNIMVTFRNQASRPYGKK | 303 |
| 22 | EEGDNIMVTFRNQASRPYGKE | 304 |
| 23 | EEGDNIMVTFRNQASRPYGEK | 305 |
| 24 | EEGDNIMVTFRNQASRPYGEE | 306 |
| 25 | KEGDNIMVTFRNQASRPYGKK | 307 |
| 26 | KEGDNIMVTFRNSQASRPYGKE | 308 |
| 27 | KEGDNIMVTFRNQASRPYGEK | 309 |
| 28 | KEGDNIMVTFRNQASRPYGEE | 310 |
| 29 | EKGDNIMVTFRNQASRPYGKK | 311 |
| 30 | EKGDNIMVTFRNQASRPYGKE | 312 |
| 31 | EKGDNIMVTFRNQASRPYGEK | 313 |
| 32 | EKGDNIMVTFRNQASRPYGEE | 314 |
| 34 | DNIMVTFRNQASRPY | 315 |
| 35 | PRCLTRYYSSFVNME | 316 |
| 36 | PRCLT | 317 |
| 37 | DNIMV | 318 |
| 38, X is | XXGDNIMVTFRNQASRPYGXX | 319 |
| K or E | | |
| 39, X is | XXGPRCLTRYYSSFVNMEGXX | 320 |
| K or E | | |
| 40 | ASQYRPSQR | 321 |

TABLE 12

| FVIII Derived | | |
|---|---|---|
| Peptides | Sequence | SEQ ID NO: |
| 1 | GTLMVFFGNVDSSGI | 322 |
| 2 | TQTLHKFILLFAVFD | 323 |
| 3 | SLYISQFIIMYSLDG | 324 |
| 3.1 | RQKFSSLYISQFIIM | 325 |
| 3.2 | QKFSSLYISQFIIMY | 326 |
| 3.3 | KFSSLYISQFIIMYS | 327 |
| 3.4 | FSSLYISQFIIMYSL | 328 |
| 3.5 | SSLYISQFIIMYSLD | 329 |
| 3.6 | SLYISQFIIMYSLDG | 330 |
| 3.7 | LYISQFIIMYSLDGK | 331 |
| 3.8 | YISQFIIMYSLDGKK | 332 |
| 3.9 | ISQFIIMYSLDGKKW | 333 |
| 3.10 | SQFIIMYSLDGKKWQ | 334 |
| 3.11 | QFIIMYSLDGKKWQT | 335 |
| 4 | PPIIARYIRLHPTHY | 336 |
| 4.1 | HNIFNPPIIARYIRL | 337 |
| 4.2 | NIFNPPIIARYIRLH | 338 |
| 4.3 | IFNPPIIARYIRLHP | 339 |
| 4.4 | FNPPIIARYIRLHPT | 340 |
| 4.5 | NPPIIARYIRLHPTH | 341 |
| 4.6 | PPIIARYIRLHPTHY | 342 |
| 4.7 | PEARYIRLHPTHYS | 343 |
| 4.8 | IIARYIRLHPTHYSI | 344 |
| 4.9 | IARYIRLHPTHYSIR | 345 |
| 4.10 | ARYERLHPTHYSIRS | 346 |
| 4.11 | RYIRLHPTHYSIRST | 347 |
| 5 | PPLLTRYLRIHPQSSW | 348 |
| 6 | MHTVNGYVNRSLPGL | 349 |
| 7 | LGQFLLFCHISSHQH | 350 |
| 8 | DTLLIIFKNQASRPY | 351 |
| 8.1 | YGEVGDTLLIIFKNQ | 352 |
| 8.2 | GEVGDTLLIIFKNQA | 353 |
| 8.3 | EVGDTLLIIFKNQAS | 354 |
| 8.4 | VGDTLLIIFKNQASR | 355 |
| 8.5 | GDTLLIIFKNQASRP | 356 |
| 8.6 | DTLLIIFKNQASRPY | 357 |
| 8.7 | TLLIIFKNQASRPYN | 358 |
| 8.8 | LLIIFKNQASRPYNI | 359 |
| 8.9 | LIIFKNQASRPYNIY | 360 |
| 8.10 | IIFKNQASRPYNIYP | 361 |
| 8.11 | IFKNQASRPYNTYPH | 362 |
| 9 | PRCLTRYYSSFVNME | 363 |
| 9.1 | PTKSDPRCLTRYYSS | 364 |
| 9.2 | TKSDPRCLTRYYSSF | 365 |
| 9.3 | KSDPRCLTRYYSSFV | 366 |
| 9.4 | SDPRCLTRYYSSFVN | 367 |
| 9.5 | DPRCLTRYYSSFVNM | 368 |
| 9.6 | PRCLTRYYSSFVNME | 369 |
| 9.7 | RCLTRYYSSFVNMER | 370 |
| 9.8 | CLTRYYSSFVNMERD | 371 |
| 9.9 | LTRYYSSFVNMERDL | 372 |
| 9.10 | TRYYSSFVNMERDLA | 373 |
| 9.11 | RYYSSFVNMERDLAS | 374 |
| 10 | TENIQRFLPNPAGVQ | 375 |
| 11 | DNIMVTFRNQASRPY | 376 |
| 11.1 | RAEVEDNIMVTFRNQ | 377 |
| 11.2 | AEVEDNIMVTFRNQA | 378 |
| 11.3 | EVEDNIMVTFRNQAS | 379 |
| 11.4 | VEDNIMVTFRNQASR | 380 |
| 11.5 | EDNTMVTFRNQASRP | 381 |
| 11.6 | DNIMVTFRNQASRPY | 382 |
| 11.7 | NIMVTFRNQASRPYS | 383 |
| 11.8 | IMVTFRNQASRPYSF | 384 |
| 11.9 | MVTFRNQASRPYSFY | 385 |
| 11.10 | VTFRNQASRPYSFYS | 386 |
| 11.11 | TFRNQASRPYSFYSS | 387 |
| 12 | RYLRIHPQSWVHQIA | 388 |
| 12.1 | PPLLTRYLRIHPQSW | 389 |
| 12.2 | PLLTRYLRIHPQSWV | 390 |
| 12.3 | LLTRYLRIHPQSWVH | 391 |
| 12.4 | LTRYLREHPQSWVHQ | 392 |
| 12.5 | TRYLRffIPQSWVHQI | 393 |
| 12.6 | RYLRIHPQSWVHQIA | 394 |
| 12.7 | YLRIHPQSWVHQIAL | 395 |
| 12.8 | LRIHPQSWVHQIALR | 396 |
| 12.9 | RIHPQSWVHQIALRM | 397 |
| 12.10 | IHPQSWVHQIALRME | 398 |
| 12.11 | HPQSWVHQIALRMEV | 399 |

TABLE 13

| FVIII Derived | | |
|---|---|---|
| Peptides | Sequence | SEQ ID NO: |
| 1 | LYISQFIIMMYSLDGK | 400 |
| 2 | FIIMYSLDGKKWQTY | 401 |
| 3 | PIIARYIRLHPTHYS | 402 |
| 4 | LIIFKNQASRPYNIY | 403 |
| 5 | LLIIFKNQASRPYNI | 404 |
| 6 | TLLIIFKNQASRPYN | 405 |
| 7 | GDTLLLIIFKNQASRP | 406 |
| 8 | VGDTLLLIIFKNQASR | 407 |
| 9 | EVGDTLLLIIFKNQAS | 408 |
| 10 | CLTRYYSSFVNMERD | 409 |
| 11 | EDNIMVTFRNQASRP | 410 |
| 12 | TRYLRIHPQSWVHQI | 411 |
| 13 | LTRYLRIHPQSWVHQ | 412 |

TABLE 14

| Autoantibody Binding | | |
|---|---|---|
| Peptides Related To | | SEQ |
| Multiple Sclerosis | Sequence | ID NO: |
| 1 | ASSRGYEDLRF | 413 |
| 2 | LDNSYHDNPV | 414 |
| 3 | LRAPAGLGAA | 415 |
| 4 | GARCINAEQP | 416 |
| 5 | YSCLKLYSFA | 417 |
| 6 | EHATQNQVSV | 418 |
| 7 | GTGSGQGEEA | 419 |
| 8 | ADDNFSIPEG | 420 |
| 9 | ASSRGYEDLR | 421 |
| 10 | LDNSYHDNPVVSKE | 422 |
| | LRIEGNQLT | |
| 11 | GARCINAEQPCQSP | 423 |
| 12 | YSCLKLYSFAN | 424 |

TABLE 15

| Myelin Basic | | |
|---|---|---|
| Protein (MBP) | | |
| Derived Peptides | Sequence | SEQ ID NO: |
| MBP 134-148 | YKSAHKGFKGVDAQG | 425 |
| MBP 135-149 | KSAHKGFKGVDAQGT | 426 |
| MBP 136-150 | SAHKGFKGVDAQGTL | 427 |
| MBP 137-151 | AHKGFKGVDAQGTLS | 428 |
| MBP 138-152 | HKGFKGVDAQGTLSK | 429 |
| MBP 139-153 | KGFKGVDAQGTLSKI | 430 |
| MBP 140-154 | GFKGVDAQGTLSKIF | 431 |

TABLE 16

| Myelin Basic | | SEQ |
|---|---|---|
| Protein (MBP) | | ID |
| Derived Peptides | Sequence | NO: |
| MBP 83-99 | ENPVVHFFKNIVTPRTP | 432 |
| MBP 80-94 | TQDENPVVHFFKNIV | 433 |
| MBP 81-95 | QDENPVVHFFKNIVT | 434 |
| MBP 82-96 | DENPVVHFFKNIVTP | 435 |
| MBP 83-97 | ENPVVHFFKNIVTPR | 436 |
| MBP 84-98 | MPVVHFFKNIVTPRT | 437 |
| MBP 130-144 | RASDYKSAHKGFKGV | 438 |
| MBP 131-145 | ASDYKSAHKGFKGVD | 439 |
| MBP 132-146 | SDYKSAHKGFKGVDA | 440 |
| MBP 133-147 | DYKSAHKGFKGVDAQ | 441 |

TABLE 17
HUMAN Myelin Oligodendrocyte Glycoprotein (MOG)
and HUMAN MOG Splice Variants*

| | | Length | | SEQ |
|---|---|---|---|---|
| | Accession | (Amino | | ID |
| Description | No. | Acids) | Sequence | NO: |
| Human | UniProtK | 247 | MASLSRPSLPSCLCSFLLLLLLQVSSSYAG | 442 |
| Isoform 1 | Q16653 | | QFRVIGPRHPIRALVGDEVELPCRISPGKN | |
| (Alpha-1) // | | | ATGMEVGWYRPPFSRVVHLYRNGKDQD | |
| Canonical | | | GDQAPEYRGRTELLKDAIGEGKVTLRIRN | |
| Sequence | | | VRFSDEGGFTCFFRDHSYQEEAAMELKVE | |
| | | | DPFYWVSPGVLVLLAVLPVLLLQITVGLIF | |
| | | | LCLQYRLRGKLRAEIENLHRTFDPHFLRVP | |
| | | | CWKITLFVIVPVLGPLVALIICYNWLHRRL | |
| | | | AGQFLEELRNPF | |
| Human | UniProtK | 208 | MASLSRPSLPSCLCSFLLLLLLQVSSSYAG | 443 |
| Isoform 2 | Q16653-2 | | QFRVIGPRHPIRALVGDEVELPCRISPGKN | |
| (Alpha-2) | | | ATGMEVGWYRPPFSRVVHLYRNGKDQD | |
| Missing | | | GDQAPEYRGRTELLKDAIGEGKVTLRIRN | |
| 198-236 | | | VRFSDEGGFTCFFRDHSYQEEAAMELKVE | |
| | | | DPFYWVSPGVLVLLAVLPVLLLQITVGLIF | |
| | | | LCLQYRLRGKLRAEIENLHRTFGQFLEELR | |
| | | | NPF | |
| Human | UniProtK | 224 | MASLSRPSLPSCLCSFLLLLLLQVSSSYAG | 444 |
| Isoform 3 | Q16653-3 | | QFRVIGPRHPIRALVGDEVELPCRISPGKN | |
| (Alpha-3) // | | | ATGMEVGWYRPPFSRVVHLYRNGKDQD | |
| 198-236: | | | GDQAPEYRGRTELLKDAIGEGKVTLRIRN | |
| DPHFLRV | | | VRFSDEGGFTCFFRDHSYQEEAAMELKVE | |
| PCWKITLF | | | DPFYWVSPGVLVLLAVLPVLLLQITVGLIF | |
| VIVPVLGP | | | LCLQYRLRGKLRAEIENLHRTFESFGVLGP | |
| LVALIICY | | | QVKEPKKTGQFLEELRNPF | |
| NWLHRRL | | | | |
| A → | | | | |
| ESFGVLGP | | | | |
| QVKEPKKT | | | | |
| Human | UniProtK | 131 | MASLSRPSLPSCLCSFLLLLLLQVSSSYAD | 445 |
| Isoform 4 | Q16653-4 | | PFYWVSPGVLVLLAVLPVLLLQITVGLIFL | |
| (Alpha-4) // | | | CLQYRLRGKLRAEIENLHRTFDPHFLRVPC | |
| Missing | | | WKITLFVIVPVLGPLVALIICYNWLHRRLA | |
| 30-145 | | | GQFLEELRNPF | |
| Human | UniProtK | 252 | MASLSRPSLPSCLCSFLLLLLLQVSSSYAG | 446 |
| Isoform 5 | Q16653-5 | | QFRVIGPRHPIRALVGDEVELPCRISPGKN | |
| (Beta-1) // | | | ATGMEVGWYRPPFSRVVHLYRNGKDQD | |
| 244-247: | | | GDQAPEYRGRTELLKDAIGEGKVTLRIRN | |
| RNPF → L | | | VRFSDEGGFTCFFRDHSYQEEAAMELKVE | |
| FHLEALS | | | DPFYWVSPGVLVLLAVLPVLLLQITVGLIF | |
| G | | | LCLQYRLRGKLRAEIENLHRTFDPHFLRVP | |
| | | | CWKITLFVIVPVLGPLVALIICYNWLHRRL | |
| | | | AGQFLEELLFHLEALSG | |
| Human | UniProtK | 213 | MASLSRPSLPSCLCSFLLLLLLQVSSSYAG | 447 |
| Isoform 6 | Q16653-6 | | QFRVIGPRHPIRALVGDEVELPCRISPGKN | |
| (Beta-2) // | | | ATGMEVGWYRPPFSRVVHLYRNGKDQD | |
| 198-236: | | | GDQAPEYRGRTELLKDAIGEGKVTLRIRN | |
| Missing. | | | VRFSDEGGFTCFFRDHSYQEEAAMELKVE | |
| 244-247: | | | DPFYWVSPGVLVLLAVLPVLLLQITVGLIF | |
| RNPF → | | | LCLQYRLRGKLRAEIENLHRTFGQFLEELL | |
| LFHLEALS | | | FHLEALSG | |
| G | | | | |
| Human | UniProtK | 229 | MASLSRPSLPSCLCSFLLLLLLQVSSSYAG | 448 |
| Isoform 7 | Q16653-7 | | QFRVIGPRHPIRALVGDEVELPCRISPGKN | |
| (Beta-3) // | | | ATGMEVGWYRPPFSRVVHLYRNGKDQD | |
| 198-236: | | | GDQAPEYRGRTELLKDAIGEGKVTLRIRN | |
| DPHFLRV | | | VRFSDEGGFTCFFRDHSYQEEAAMELKVE | |
| PCWKITLF | | | DPFYWVSPGVLVLLAVLPVLLLQITVGLIF | |
| VIVPVLGP | | | LCLQYRLRGKLRAEIENLHRTFESFGVLGP | |
| LVALIICY | | | QVKEPKKTGQFLEELLFHLEALSG | |
| NWLHRRL | | | | |
| A → ESFG | | | | |
| VLGPQVK | | | | |
| EPKKT | | | | |
| 244-247: | | | | |
| RNPF → L | | | | |
| FHLEALS | | | | |
| G | | | | |
| Human | UniProtK | 206 | MASLSRPSLPSCLCSFLLLLLLQVSSSYAG | 449 |
| Isoform 8 | Q16653-8 | | QFRVIGPRHPIRALVGDEVELPCRISPGKN | |
| (Beta-4) // | | | ATGMEVGWYRPPFSRVVHLYRNGKDQD | |
| 198-243: | | | GDQAPEYRGRTELLKDAIGEGKVTLRIRN | |
| Missing. | | | VRFSDEGGFTCFFRDHSYQEEAAMELKVE | |
| 244-247: | | | DPFYWVSPGVLVLLAVLPVLLLQITVGLIF | |
| RNPF → L | | | LCLQYRLRGKLRAEIENLHRTFLFHLEALS | |
| FHLEALS | | | G | |
| G | | | | |
| Human | UniProtK | 203 | MASLSRPSLPSCLCSFLLLLLLQVSSSYAG | 450 |
| Isoform 9 // | Q16653-9 | | QFRVIGPRHPIRALVGDEVELPCRISPGKN | |
| 198-203: | | | ATGMEVGWYRPPFSRVVHLYRNGKDQD | |
| DPHFLR | | | GDQAPEYRGRTELLKDAIGEGKVTLRIRN | |
| GKFRHV | | | VRFSDEGGFTCFFRDHSYQEEAAMELKVE | |
| 204-247: | | | DPFYWVSPGVLVLLAVLPVLLLQITVGLIF | |
| Missing | | | LCLQYRLRGKLRAEIENLHRTFGKFRHV | |
| Human | UniProtK | 171 | MASLSRPSLPSCLCSFLLLLLLQVSSSYAG | 451 |
| Isoform 10 // | Q16653-10 | | QFRVIGPRHPIRALVGDEVELPCRISPGKN | |
| 146-171: | | | ATGMEVGWYRPPFSRVVHLYRNGKDQD | |
| DPFYWVS | | | GDQAPEYRGRTELLKDAIGEGKVTLRIRN | |
| PGVLVLL | | | VRFSDEGGFTCFFRDHSYQEEAAMELKVE | |
| AVLPVLL | | | VSHSVTQDWLQWHDHGSLQPPPPRLK | |
| LQITV → | | | | |
| VSHSVTQ | | | | |
| DWLQWH | | | | |
| DHGSLQP | | | | |
| PPPRLK | | | | |
| 172-247: | | | | |
| Missing. | | | | |
| Human | UniProtK | 206 | MASLSRPSLPSCLCSFLLLLLLQVSSSYAG | 452 |
| Isoform 11 // | Q16653-11 | | QFRVIGPRHPIRALVGDEVELPCRISPGKN | |
| 198-247: | | | ATGMEVGWYRPPFSRVVHLYRNGKDQD | |
| DPHFLRV | | | GDQAPEYRGRTELLKDAIGEGKVTLRIRN | |
| PCWKITLF | | | VRFSDEGGFTCFFRDHSYQEEAAMELKVE | |
| VIVPVLGP | | | DPFYWVSPGVLVLLA VLPVLLLQITVGLIF | |
| LVALIICY | | | LCLQYRLRGKLRAEIENLHRTFVFHLEALS | |
| NWLHRRL | | | G | |
| AGQFLEE | | | | |
| LRNPF → | | | | |
| VFHLEAL | | | | |
| SG | | | | |
| Human | UniProtK | 108 | MASLSRPSLPSCLCSFLLLLLLQVSSSYAD | 453 |
| Isoform 12 // | Q16653-12 | | PFYWVSPGVLVLLAVLPVLLLQITVGLIFL | |
| 30-145: | | | CLQYRLRGKLRAEIENLHRTFESFGVLGPQ | |
| Missing. | | | VKEPKKTGQFLEELRNPF | |
| 198-236: | | | | |
| DPHFLRV | | | | |
| PCWKITLF | | | | |
| VIVPVLGP | | | | |
| LVALIICY | | | | |
| NWLHRRL | | | | |
| A → ESFG | | | | |
| VLGPQVK | | | | |
| EPKKT | | | | |
| Human | UniProtK | 295 | MASLSRPSLPSCLCSFLLLLLLQVSSSYAG | 454 |
| Isoform 13 // | Q16653-13 | | QFRVIGPRHPIRALVGDEVELPCRISPGKN | |
| 245-247: | | | ATGMEVGWYRPPFSRVVHLYRNGKDQD | |
| NPF → KF | | | GDQAPEYRGRTELLKDAIGEGKVTLRIRN | |
| SSLCYKQ | | | VRFSDEGGFTCFFRDHSYQEEAAMELKVE | |
| RIKSQERE | | | DPFYWVSPGVLVLLAVLPVLLLQITVGLIF | |
| TEATRGR | | | LCLQYRLRGKLRAEIENLHRTFDPHFLRVP | |
| GGLLRDH | | | CWKITLFVIVPVLGPLVALIICYNWLHRRL | |
| IPRGKEEL | | | AGQFLEELRKFSSLCYKQRIKSQERETEAT | |
| ESLGGGK | | | RGRGGLLRDHIPRGKEELESLGGGKTPPG | |
| TPPGR | | | R | |
| Human | GenBank | 90 | SLSRPSLPSCLCSFLLLLLLQVSSSYADPFY | 455 |
| Alpha-5 // | AY566846 | | WVSPGVLVLLAVLPVLLLQITVGLVFLCL | |
| Exon 2 and | | | QYRLRGKLRAEIENLHRTFGQFLEELRNPF | |
| 6B deletion | | | | |
| Human | GenBank | 106 | SLSRPSLPSCLCSFLLLLLLQVSSSYADPFY | 456 |
| Alpha-6 // | AY566847 | | WVSPGVLVLLAVLPVLLLQITVGLVFLCL | |
| exon 2 | | | QYRLRGKLRAEIENLHRTFESFGVLGPQV | |
| deletion and | | | KEPKKTGQFLEELRNPF | |
| alternative | | | | |
| 6A | | | | |
| MOG Ig- | GenBank | 171 | SLSRPSLPSCLCSFLLLLLLQVSSSYAGQFR | 457 |
| Alu-10B | AY566848 | | VIGPRHPIRALVGDEVELPCRISPGKNATG | |
| | | | MEVGWYRPPFSRVVHLYRNGKDQDGDQ | |
| | | | APEYRGRTELLKDAIGEGKVTLRIRNVRFS | |
| | | | DEGGFTCFFRDHSYQEEAAMELKVEGLV | |
| | | | AVARSWLIAASTSQAQVILLTQPLE | |
| MOG Alu- | GenBank | 53 | SLSRPSLPSCLCSFLLLLLLQVSSSYAVSHS | 458 |
| 10A | AY566849 | | VTQDWLQWHDHGSLQPPPPRLK | |
| MOG Alu- | GenBank | 55 | SLSRPSLPSCLCSFLLLLLLQVSSSYAGLVA | 459 |
| 10B | AY566850 | | VARSWLIAASTSQAQVILLTQPLE | |
| MOG Ig2′ | GenBank | 151 | SLSRPSLPSCLCSFLLLLLLQVSSSYAGQFR | 460 |
| | AY566851 | | VIGPRHPIRALVGDEVELPCRISPGKNATG | |
| | | | MEVGWYRPPFSRVVHLYRNGKDQDGDQ | |
| | | | APEYRGRTELLKDAIGEGKVTLRIRNVRFS | |
| | | | DEGGFTCFFRDHSYQEEAAMELKVEAPLK | |
| | | | QRSR | |
| MOG Ig- | | | Sequence Not Shown | |
| Alu-10A | | | Isoform comprising exons 1, 2 | |
| | | | and Alu-10^{A} | |

*Additional splice variants can be found in WO/2007/008933A2

### ^{A}See Delarasse et al., 2006, J. Neurochem., 98, 1707-1717.

### Example 5

Spk1 and Spk2 VPI capsid protein sequences and CAG promoter sequence

Spk1 VP1 capsid (SEQ ID NO:1):

MAADGYLPDWLEDNLSEGIREWWDLKPGAPKPKAN
QQKQDNGRGLVLPGYKYLGPFNGLDKGEPVNAADA
AALEHDKAYDQQLQAGDNPYLRYNHADAEFQERLQ
EDTSFGGNLGRAVFQAKKRVLEPLGLVESPVKTAP
GKKRPVEPSPQRSPDSSTGIGKKGQQPAKKRLNFG
QTGDSESVPDPQPIGEPPAAPSGVGPNTMAAGGGA
PMADNNEGADGVGSSSGNWHCDSTWLGDRVITTST
RTWALPTYNNHLYKQISNGTSGGSTNDNTYFGYST
PWGYFDFNRFHCHFSPRDWQRLINNNWGFRPKRLN
FKLFNIQVKEVTQNEGTKTIANNLTSTIQVFTDSE
YQLPYVLGSAHQGCLPPFPADVFMIPQYGYLTLNN
GSQAVGRSSFYCLEYFPSQMLRTGNNFEFSYNFED
VPFHSSYAHSQSLDRLMNPLIDQYLYYLSRTQSTG
GTAGTQQLLFSQAGPNNMSAQAKNWLPGPCYRQQR
VSTTLSQNNNSNFAWTGATKYHLNGRDSLVNPGVA
MATHKDDEERFFPSSGVLMFGKQGAGKDNVDYSSV
MLTSEEEIKTTNPVATEQYGVVADNLQQQNAAPIV
GAVNSQGALPGMVWQNRDVYLQGPIWAKIPHTDGN
FHPSPLMGGFGLKHPPPQILIKNTPVPADPPTTFN
QAKLASFITQYSTGQVSVEIEWELQKENSKRWNPE
IQYTSNYYKSTNVDFAVNTEGTYSEPRPIGTRYLT
RNL

Spk2 VP1 capsid (SEQ ID NO:2):

MAADGYLPDWLEDNLSEGIREWWALQPGAPKPKAN
QQHQDNARGLVLPGYKYLGPGNGLDKGEPVNAADA
AALEHDKAYDQQLKAGDNPYLKYNHADAEFQERLK
EDTSFGGNLGRAVFQAKKRLLEPLGLVEEAAKTAP
GKKRPVDQSPQEPDSSSGVGKSGKQPARKRLNFGQ
TGDSESVPDPQPLGEPPAAPTSLGSNTMASGGGAP
MADNNEGADGVGNSSGNWHCDSQWLGDRVITTSTR
TWALPTYNNHLYKQISSQSGASNDNHYFGYSTPWG
YFDFNRFHCHFSPRDWQRLINNNWGFRPKKLSFKL
FNIQVKEVTQNDGTTTIANNLTSTVQVFTDSEYQL
PYVLGSAHQGCLPPFPADVFMVPQYGYLTLNNGSQ
AVGRSSFYCLEYFPSQMLRTGNNFQFSYTFEDVPF
HSSYAHSQSLDRLMNPLIDQYLYYLNRTQGTTSGT
TNQSRLLFSQAGPQSMSLQARNWLPGPCYRQQRLS
KTANDNNNSNFPWTAASKYHLNGRDSLVNPGPAMA
SHKDDEEKFFPMHGNLIFGKEGTTASNAELDNVMI
TDEEEIRTTNPVATEQYGTVANNLQSSNTAPTTRT
VNDQGALPGMVWQDRDVYLQGPIWAKIPHTDGHFH
PSPLMGGFGLKHPPPQIMIKNTPVPANPPTTFSPA
KFASFITQYSTGQVSVEIEWELQKENSKRWNPEIQ
YTSNYNKSVNVDFTVDTNGVYSEPRPIGTRYLTRP
L

CAG Promoter Sequence (SEQ ID NO:3):

ATAGCCCATATATGGAGTTCCGCGTTACATAACTT
ACGGTAAATGGCCCGCCTGGCTGACCGCCCAACGA
CCCCCGCCCATTGACGTCAATAATGACGTATGTTC
CCATAGTAACGCCAATAGGGACTTTCCATTGACGT
CAATGGGTGGAGTATTTACGGTAAACTGCCCACTT
GGCAGTACATCAAGTGTATCATATGCCAAGTACGC
CCCCTATTGACGTCAATGACGGTAAATGGCCCGCC
TGGCATTATGCCCAGTACATGACCTTATGGGACTT
TCCTACTTGGCAGTACATCTACGTATTAGTCATCG
CTATTACCATGGTCGAGGTGAGCCCCACGTTCTGC
TTCACTCTCCCCATCTCCCCCCCCTCCCCACCCCC
AATTTTGTATTTATTTATTTTTTAATTATTTTGTG
CAGCGATGGGGGCGGGGGGGGGGGGGGGGCGCGCG
CCAGGCGGGGCGGGGCGGGGCGAGGGGCGGGGCGG
GGCGAGGCGGAGAGGTGCGGCGGCAGCCAATCAGA
GCGGCGCGCTCCGAAAGTTTCCTTTTATGGCGAGG
CGGCGGCGGCGGCGGCCCTATAAAAAGCGAAGCGC
GCGGCGGGCGGGGAGTCGCTGCGACGCTGCCTTCG
CCCCGTGCCCCGCTCCGCCGCCGCCTCGCGCCGCC
CGCCCCGGCTCTGACTGACCGCGTTACTCCCACAG
GTGAGCGGGCGGGACGGCCCTTCTCCTCCGGGCTG
TAATTAGCGCTTGGTTTAATGACGGCTTGTTTCTT
TTCTGTGGCTGCGTGAAAGCCTTGAGGGGCTCCGG
GAGGGCCCTTTGTGCGGGGGGAGCGGCTCGGGGGG
TGCGTGCGTGTGTGTGTGCGTGGGGAGCGCCGCGT
GCGGCTCCGCGCTGCCCGGCGGCTGTGAGCGCTGC
GGGCGCGGCGCGGGGCTTTGTGCGCTCCGCAGTGT
GCGCGAGGGGAGCGCGGCCGGGGGCGGTGCCCCGC
GGTGCGGGGGGGGCTGCGAGGGGAACAAAGGCTGC
GTGCGGGGTGTGTGCGTGGGGGGGTGAGCAGGGGG
TGTGGGCGCGTCGGTCGGGCTGCAACCCCCCCTGC
ACCCCCCTCCCCGAGTTGCTGAGCACGGCCCGGCT
TCGGGTGCGGGGCTCCGTACGGGGCGTGGCGCGGG
GCTCGCCGTGCCGGGCGGGGGGTGGCGGCAGGTGG
GGGTGCCGGGCGGGGCGGGGCCGCCTCGGGCCGGG
GAGGGCTCGGGGGAGGGGCGCGGCGGCCCCCGGAG
CGCCGGCGGCTGTCGAGGCGCGGCGAGCCGCAGCC
ATTGCCTTTTATGGTAATCGTGCGAGAGGGCGCAG
GGACTTCCTTTGTCCCAAATCTGTGCGGAGCCGAA
ATCTGGGAGGCGCCGCCGCACCCCCTCTAGCGGGC
GCGGGGCGAAGCGGTGCGGCGCCGGCAGGAAGGAA
ATGGGCGGGGAGGGCCTTCGTGCGTCGCCGCGCCG
CCGTCCCCTTCTCCCTCTCCAGCCTCGGGGCTGTC
CGCGGGGGGACGGCTGCCTTCGGGGGGGACGGGGC
AGGGCGGGGTTCGGCTTCTGGCGTGTGACCGGCGG
CTCTAGAGCCTCTGCTAACCATGTTCATGCCTTCT
TCTTTTTCCTACAGCTCCTGGGCAACGTGCTGGTT
ATTGTGCTGTCTCATCATTTTGGCAAA

### Example 6

Prevention of Experimental Autoimmune Encephalomyelitis (EAE) Via Liver Mediated AAV Immunotherapy

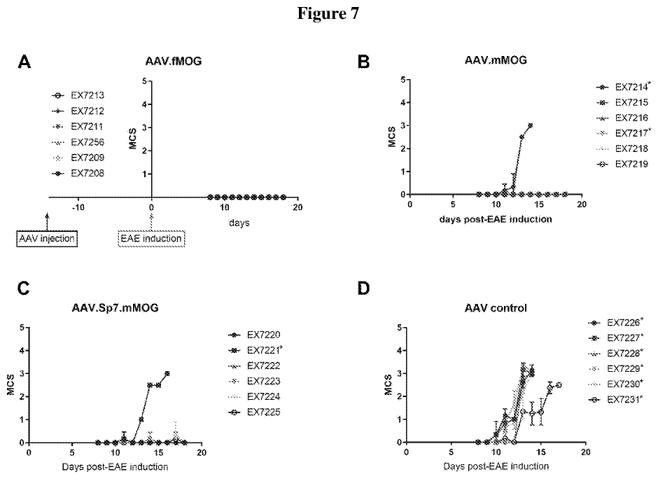

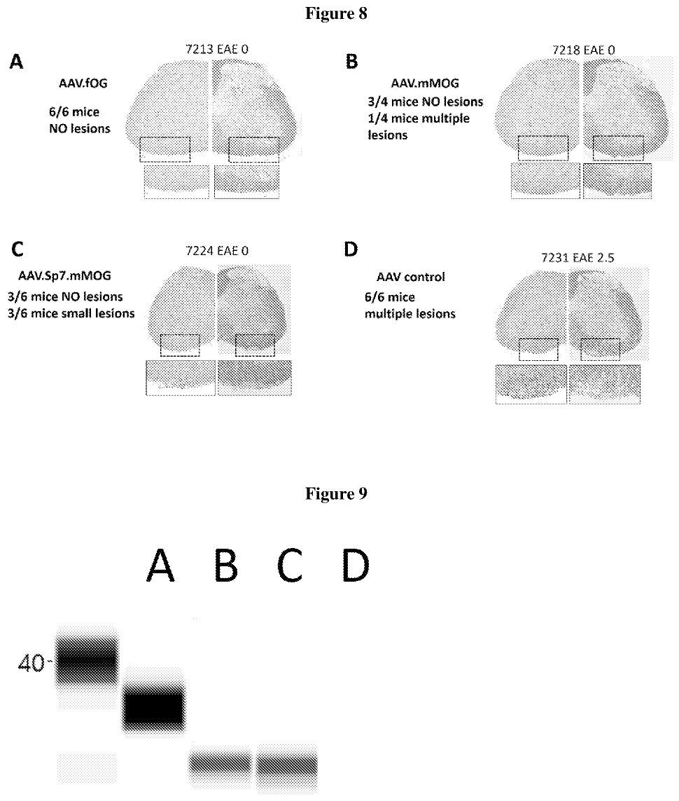

Eight to nine week-old juvenile C57B/6 mice were IV injected via the tail vein with 1E11vgs of AAV.ApoE/hAAT vectors to prevent EAE clinical disease (FIG. 6). Vectors contained either cDNA encoding for full-length murine myelin oligodendrocyte glycoprotein (fMOG), a shortened version of fMOG termed mini-MOG (mMOG), mMOG with a secretion signal (human chymotrypsinogen B2 signal peptide; “Sp7”) (Sp7.mMOG) (polynucleotide SEQ ID NO: 462, encoding protein SEQ ID NO: 470), or an unrelated control vector (control). Treated mice and control groups were followed for 2 weeks before induction of MOG_{35_55}-EAE. Mice were sacrificed when control groups reached a mean clinical score (MCS; see, e.g., Keeler et al., Mol Ther. 2018 Jan. 3; 26(1):173-183) of 3.0-3.5 at ˜18 days post-EAE induction. All mice (N=6) pre-treated with AAV.ApoE/hAAT.fMOG vectors remained free of clinical disease (MCS=0, N=6) for the entire study (FIG. 7A). Four mice in the AAV.mMOG treated group (N=6) never developed clinical EAE (FIG. 7B) while five mice in the secreted mMOG group (FIG. 7C; Sp7.mMOG, N=6) also remained free from clinical disease. Mice treated with control vectors developed clinical disease as expected (FIG. 7D). Histological analysis corresponds with clinical disease where multiple infiltrating lesions were identified in control vector mice that succumbed to EAE (FIG. 8D) and no lesions were noted in AAV.fMOG animals (FIG. 8A). Shortened MOG vectors protected mice from clinical disease, but indications of pathological disease were seen in mMOG treated mice as small infiltrating lesions were noted in specimens without clinical EAE development (FIG. 8B&C). Liver homogenates were run on a WES 12-230 kDa and probed with goat anti-mouse MOG. MOG protein was identified in all samples except for AAV controls vectors, which was expected as this vector had no MOG transgene (FIG. 9).

### Example 7

Rescue from Clinical EAE Progression in Mice Treated with AAV+/−Rapamycin Post-EAE Exposure

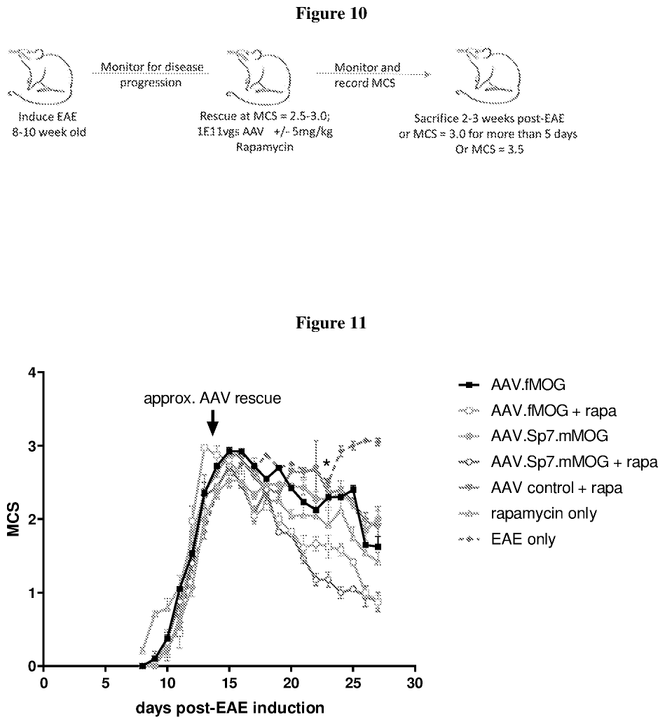

EAE was induced in eight to ten week-old juvenile C57B/6 mice and clinical progression was recorded via MCS. When animals reached MCS=2.5-3.0 they were randomly assigned to one of the following rescue treatment groups: AAV.fMOG, AAV.fMOG+rapamycin (rapa), AAV.Sp7.mMOG, AAV.Sp7.mMOG+rapa, AAV.control+rapa, rapa only, or no treatment (EAE only). Rapamycin was given intraperitoneally (IP) at 0, 48, and 96 hours post-rescue. AAV was dosed at 1E11vgs total and given IV tail vein. Groups were then followed clinically for an additional two weeks post-rescue (FIG. 10). All treated groups showed improvement compared to the untreated control group, EAE only, as clinical signs improved two-weeks post-rescue (FIG. 11). Groups treated with AAV alone, either AAV.fMOG or AAV.Sp7.mMOG, experienced similar final MCS scores as the rapamycin control groups. However, groups treated with both AAV vectors and rapamycin displayed lower MCS scores when given together (FIG. 6, AAVf.MOG+rapa and AAV.Sp7.mMOG+rapa).

What is claimed is:

1. An expression cassette comprising an expression control element operably linked to a nucleic acid encoding a fusion protein, said fusion protein comprising an unwanted antigen and a leader sequence for cell secretion.

2. The expression cassette of claim 1, wherein said unwanted antigen comprises a self-antigen, autoantigen or protein or peptide that has structural similarity or sequence identity to said self-antigen or said autoantigen.

3. The expression cassette of claim 1, wherein said protein or peptide that has structural similarity or sequence identity to said self-antigen or said autoantigen is a microbial protein or peptide.

4. The expression cassette of claim 1, wherein said unwanted antigen comprises an allergen.

5. The expression cassette of claim 4, wherein said allergen comprises a plant, insect, or animal allergen.

6. The expression cassette of any one of claims 1-5, wherein said unwanted antigen is not a protein or peptide for correcting or replacing a defective or unexpressed gene or protein in a subject.

7. The expression cassette of any one of claims 1-5, wherein said nucleic acid does not comprise a gene for replacing a defective or unexpressed gene or protein in a subject.

8. The expression cassette of claim 1, wherein said unwanted antigen binds to or activates T regulatory cells (Tregs) when expressed in a subject.

9. The expression cassette of claim 8, wherein said Tregs are Fox P3+/CD4+/CD25+Tregs.

10. The expression cassette of claim 1, wherein said unwanted antigen causes exhaustion or deletion of effector T cells when expressed in a subject.

11. The expression cassette of any one of claims 1-10, wherein said unwanted antigen is truncated or is a subsequence of a full length native/wildtype unwanted antigen.

12. The expression cassette of any one of claims 1-11, wherein said unwanted antigen is an immune tolerizing unwanted antigen.

13. The expression cassette of any one of claims 1-12, wherein said leader sequence comprises or consists of an amino acid sequence of any of SEQ ID NOs: 13-25.

14. The expression cassette of any one of claims 1-13, wherein said unwanted antigen comprises a mammalian protein or peptide.

15. The expression cassette of any one of claims 1-14, wherein said unwanted antigen comprises a human protein or peptide.

16. The expression cassette of any one of claims 1-15, wherein said unwanted antigen comprises an antigen having or consisting of the amino acid sequence of any of SEQ ID NOs: 5-8, 51-460, 463-469, 477-484, or a subsequence of any of SEQ ID NOs: 5-8, 51-460, 463-469, or 477-484 capable of inducing an immune response in a subject when expressed in the subject.

17. The expression cassette of any one of claims 1-15, wherein said unwanted antigen comprises a myelin oligodendrocyte glycoprotein (MOG), myelin basic protein (MBP), proteolipid protein (PLP), or subsequence thereof.

18. The expression cassette of claim 17, wherein said MOG lacks all or a part of its transmembrane domain.

19. The expression cassette of claim 17, wherein said MOG comprises or consists of amino acids 1-117 of a mature MOG.

20. The expression cassette of claim 17, wherein said MOG subsequence is a subsequence of an extracellular domain or a subsequence of a transmembrane domain.

21. The expression cassette of claim 17, wherein said MOG comprises or consists of amino acids 35-55, 118-132, 181-195, or 186-200 of a mature MOG.

22. The expression cassette of claim 17, wherein said MOG comprises or consists of amino acids 1-20, 11-30, 21-40, 31-50, etc. of a mature MOG.

23. The expression cassette of claim 17, wherein said MOG comprises or consists of the amino acid sequence of any of SEQ ID NOs: 5-8, 246-251, 442-460, and 463-469, or a subsequence thereof capable of inducing an immune response in a subject when expressed in the subject.

24. The expression cassette of claim 17, wherein said MBP includes a transmembrane domain.

25. The expression cassette of claim 17, wherein said MBP lacks all or a part of a transmembrane domain.

26. The expression cassette of claim 17, wherein said MBP subsequence is a subsequence of an extracellular domain or a subsequence of a transmembrane domain of an MBP.

27. The expression cassette of claim 17, wherein said PLP lacks all or a part of a transmembrane domain.

28. The expression cassette of claim 17, wherein said PLP subsequence is a subsequence of an extracellular domain or a subsequence of a transmembrane domain of a PLP.

29. The expression cassette of claim 17, wherein said PLP comprises or consists of amino acids 37-63, 89-151, 178-233, or 261-277 of mature PLP.

30. The expression cassette of any one of claims 1-29, further comprising one or more additional polynucleotide elements positioned 5′ and/or 3′ of said nucleic acid or expression control element.

31. The expression cassette of any one of claims 1-30, wherein said expression control element is positioned 5′ of said nucleic acid.

32. The expression cassette of any one of claims 1-31, wherein said expression control element comprises an ApoE/hAAT enhancer/promoter sequence, a CAG promoter, cytomegalovirus (CMV) immediate early promoter/enhancer, Rous sarcoma virus (RSV) promoter/enhancer, SV40 promoter, dihydrofolate reductase (DHFR) promoter, or chicken β-actin (CBA) promoter.

33. The expression cassette of any one of claims 1-32, further comprising a poly-adenylation sequence positioned 3′ of said nucleic acid.

34. The expression cassette of any one of claims 1-33, further comprising an intron, said intron optionally positioned between said expression control element and said nucleic acid or optionally positioned within said nucleic acid.

35. The expression cassette of any one of claims 1-34, wherein said expression cassette is positioned between one or more 5′ and/or 3′adeno-associated virus (AAV) inverted terminal repeat(s) (ITR(s)).

36. The expression cassette of claim 35, wherein said one or more 5′ and/or 3′ ITR(s) comprise AAV serotype AAV1, AAV2, AAV3, AAV4, AAV5, AAV6, AAV7, AAV8, AAV9, AAV10, AAV11, AAV12, AAV-2i8, AAV3B, Rh74 or Rh10 ITR.

37. The expression cassette of claim 35, wherein said AAV ITR(s) comprises a mutated, modified or variant AAV ITR that is not processed by AAV Rep protein.

38. The expression cassette of claim 37, wherein said AAV ITR(s) comprises a mutated, modified or variant AAV ITR that allows or facilitates formation of a self-complementary expression cassette.

39. The expression cassette of claim 37 or 38, wherein said mutated, modified or variant AAV ITR has a deleted D sequence, and/or a mutated, modified or variant terminal resolution site (TRS) sequence.

40. The expression cassette of any one of claims 1-39, wherein said nucleic acid, expression control element, poly-adenylation sequence or ITR has reduced CpG dinucleotides.

41. The expression cassette of any one of claims 1-40, wherein said nucleic acid, expression control element, poly-adenylation sequence or ITR has increased CpG dinucleotides.

42. A viral particle comprising said expression cassette of any one of claims 1-41.

43. A lentiviral particle comprising said expression cassette of any one of claims 1-41.

44. A lipid nanoparticle (LNP) composition comprising said expression cassette of any one of claims 1-41.

45. A recombinant adeno associated virus (rAAV) particle comprising said expression cassette of any one of claims 1-41.

46. The rAAV particle of claim 45, wherein said expression cassette comprises in 5′→3′ orientation a first AAV ITR; a promoter operable in mammalian cells; said nucleic acid; a polyadenylation signal; and optionally a second AAV ITR.

47. The rAAV particle of claim 45, wherein said rAAV particle comprises a VP1, VP2 or VP3 sequence 60% or more identical to a VP1, VP2 or VP3 sequence of AAV serotype AAV1, AAV2, AAV3, AAV4, AAV5, AAV6, AAV7, AAV8, AAV9, AAV10, AAV11, AAV12, AAV-2i8, AAV3B, Rh74, Rh10, SPK1 (SEQ ID NO:1), SPK2 (SEQ ID NO:2) VP1, VP2 and/or VP3, or a hybrid or chimera of any of the foregoing AAV serotypes.

48. The rAAV particle of claim 45, wherein said rAAV particle comprises VP1, VP2 and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV10, AAV11, AAV12, AAV-2i8, AAV3B, Rh10, Rh74, SPK1 (SEQ ID NO:1) and SPK2 (SEQ ID NO:2) VP1, VP2 and/or VP3 capsid proteins.

49. A pharmaceutical composition comprising one or more of particles of any one of claims 42-48 in a biologically compatible carrier or excipient.

50. The pharmaceutical composition of claim 49, further comprising empty AAV capsids.

51. The pharmaceutical composition of claim 50, wherein the ratio of said empty AAV capsids to said rAAV particle is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100.

52. The pharmaceutical composition of claim 50, wherein the ratio of said empty AAV capsids to said rAAV particle is about 2:1, 3:1, 4:1, 5:1, 6:1, 7:1, 8:1, 9:1, or 10:1.

53. The pharmaceutical composition of any one of claims 49-52, further comprising a surfactant.

54. A lipid nanoparticle (LNP) composition comprising said rAAV particle of any one of claims 45-48.

55. A method of suppressing, reducing or inhibiting a cell-mediated or antibody mediated immune response to an unwanted antigen in a mammal, comprising:
(a) providing said expression cassette of any one of claims 1-41, particle of any one of claims 42-48, or pharmaceutical composition or LNP composition of any one of claims 49-54; and
(b) administering an amount of said expression cassette, particle, pharmaceutical or LNP composition to said mammal, wherein said fusion protein is expressed in said mammal sufficient to suppress, reduce or inhibit a cell-mediated or antibody mediated immune response to said unwanted antigen.

56. A method of inducing tolerance in a mammal to an unwanted antigen, comprising:
(a) providing said expression cassette of any one of claims 1-41, particle of any one of claims 42-48, or pharmaceutical composition or LNP composition of any one of claims 49-54; and
(b) administering an amount of said expression cassette, particle, pharmaceutical or LNP composition to said mammal, wherein said fusion protein is expressed in said mammal sufficient to induce tolerance to said unwanted antigen.

57. A method of treating a human in need of said fusion protein, comprising:
(a) providing said expression cassette of any one of claims 1-41, particle of any one of claims 42-48, or pharmaceutical composition or LNP composition of any one of claims 49-54; and
(b) administering an amount of said expression cassette, particle, pharmaceutical or LNP composition to said human, wherein said fusion protein is expressed in said human.

58. The method of claim 57, wherein said human has an autoimmune disease or disorder.

59. The method of claim 57, wherein said human has an allergy or allergic disease or disorder.

60. The method of claim 57, wherein said human has a disease or disorder set forth in any of Tables 1 and 2.

61. The method of claim 57, wherein said human has multiple sclerosis, anti-MAG peripheral neuropathy, type 1 diabetes, Graves disease, rheumatoid arthritis, proteoglycan induced arthritis (PGIA) or myasthenia gravis.

62. The method of any one of claims 55-61, wherein said administering is intravenous, intra-arterial, intra-cavity, intra-mucosal, or via catheter.

63. The method of any one of claims 55-62, wherein said rAAV particle is administered in a range from about 1×10⁸ to about 1×10¹⁴ AAV vector genomes per kilogram (vg/kg) of the weight of said human.

64. The method of any one of claims 55-63, further comprising administering an immunosuppressive agent.

65. The method of claim 64, wherein said immunosuppressive agent comprises an anti-inflammatory agent.

66. The method of claim 64, wherein said immunosuppressive agent is a steroid.

67. The method of claim 64, wherein said immunosuppressive agent comprises rapamycin, a cyclosporine (e.g., cyclosporine A), mycophenolate, rituximab or a derivative thereof.

68. The method of any one of claims 55-67, wherein said method reduces, decreases or inhibits one or more symptoms of said auto immune disease or disorder or allergy or allergic disorder.

69. A cell comprising the expression cassette of any one of claims 1-41.

70. A cell that produces the viral particle, lentiviral particle or rAAV particle of any one of claims 42-48.

71. A method of producing a plurality of rAAV particles of any one of claims 42-48, comprising
a. introducing an AAV vector genome comprising said expression cassette of any one of claims 1-41 into a packaging helper cell; and
b. culturing said helper cell under conditions to produce said rAAV particles.

72. A method of producing a plurality of rAAV particles of any one of 42-48, comprising
a. introducing said expression cassette of any one of claims 1-40 into a packaging helper cell; and
b. culturing said helper cells under conditions to produce said rAAV particles.

73. The method of claim 71 or 72, further comprising isolating or purifying said rAAV particles.

74. The cell or method of any one of claims 69-72, wherein said cell comprises mammalian cells.

75. The cell or method of any one of claims 69-72, wherein said cell provides helper functions that package said AAV vector genome into said rAAV particle.

76. The cell or method of any one of claims 69-72, wherein said cell provides AAV helper functions.

77. The cell or method of any one of claims 69-72, wherein said cell provides AAV Rep and/or Cap proteins.

78. The cell or method of any one of claims 69-72, wherein said cell is stably or transiently transfected with polynucleotide(s) encoding AAV Rep and/or Cap protein sequence(s).

79. The cell or method of any one of claims 69-72, wherein said cell comprises HEK-293 cells.


SEQUENCE LISTING (492 sequences)

SEQ ID NO: 1 - protein, 738 aa, Artificial Sequence
  Spk1 VP1 capsid
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Artificial Sequence
  Spk2 VP1 capsid
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - DNA, 1672 bp, Artificial Sequence
  CAG Promoter Sequence
atagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 4 - DNA, 749 bp, Mus musculus
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctctgtaggccttgtattcctcttcctgcagcacagactgagaggaaaacttcgtgcagaagtagagaatctccatcggacttttgatcctcacttcctgagggtgccctgctggaagataacactgtttgttattgtgcctgttcttggacccctggttgccttgatcctgtgctacaactggctgcaccgaagactggcaggacagtttcttgaagagctaagaaaccccttttgaagatc

SEQ ID NO: 5 - protein, 247 aa, Mus musculus
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVSVGLVFLFLQHRLRGKLRAEVENLHRTFDPHFLRVPCWKITLFVIVPVLGPLVALILCYNWLHRRLAGQFLEELRNPF

SEQ ID NO: 6 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 7 - protein, 247 aa, Macaca mulatta
MASLSRPSLPSCLCSFLLLLLLQVSSSYAGQFRVIGPRQPIRALVGDEVELPCRISPGKNATGMEVGWYRPPFSRVVHLYRNGRDQDGEQAPEYRGRTELLKDAIGEGKVTLRIRNVRFSDEGGFTCFFRDHSYQEEAAIELKVEDPFYWVSPAVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 8 - protein, 245 aa, Callithrix jacchus
MASLSKPSLPSYLCFLLLLLHVSSSYGGQFRVIGPSHPIQALVGDAAELPCRISPGKNATGMEVGWYRSPFSRVVHLYRNGKDQDGEQAPEYRGRTELLKDDIGEGKVTLKIRNVRFPDEGGFTCFFRDHSYQEEAAMQLKVEDPFYWVSPGVLVLLAVLPVLFLQITVGLVFLYLQHRLRGKLRAEIENLHRTFDPHFLRVPCWKITLFVIVPVLGPLVALIICYNWLHRRLAGQFLEELRNPF

SEQ ID NO: 9 - DNA, 474 bp, Artificial Sequence
  Native leader + Mus musculus mini-MOG cDNA
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgacttga

SEQ ID NO: 10 - DNA, 446 bp, Artificial Sequence
  human chymotrypsinogen B2 signal peptide + Mus musculus mini-MOG cDNA
atggcctttctgtggctgctgtcctgctgggccctgctggggaccacctttggcggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgacttgaagatc

SEQ ID NO: 11 - DNA, 444 bp, Artificial Sequence
  HC7 leader + Mus musculus mini-MOG cDNA
atggagtttgggctgagctgggttttcctcgttgctctttttagaggtgtccagtgtggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgacttga

SEQ ID NO: 12 - DNA, 444 bp, Artificial Sequence
  Gaussia leader + Mus musculus mini-MOG cDNA
atgggagtcaaagttctgtttgccctgatctgcatcgctgtggccgaggccaagcccggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgacttga

SEQ ID NO: 13 - protein, 29 aa, Artificial Sequence
  Native mouse MOG leader sequence
MACLWSFSWPSCFLSLLLLLLLQLSCSYA

SEQ ID NO: 14 - protein, 19 aa, Artificial Sequence
  HC1 leader sequence
MELGLSWIFLLAILKGVQC

SEQ ID NO: 15 - protein, 19 aa, Artificial Sequence
  HC2 leader sequence
MELGLRWVFLVAILEGVQC

SEQ ID NO: 16 - protein, 19 aa, Artificial Sequence
  HC3 leader sequence
MKHLWFFLLLVAAPRWVLS

SEQ ID NO: 17 - protein, 19 aa, Artificial Sequence
  HC4 leader sequence
MDWTWRILFLVAAATGAHS

SEQ ID NO: 18 - protein, 19 aa, Artificial Sequence
  HC5 leader sequence
MDWTWRFLFVVAAATGVQS

SEQ ID NO: 19 - protein, 19 aa, Artificial Sequence
  HC6 leader sequence
MEFGLSWLFLVAILKGVQC

SEQ ID NO: 20 - protein, 19 aa, Artificial Sequence
  HC7 leader sequence
MEFGLSWVFLVALFRGVQC

SEQ ID NO: 21 - protein, 26 aa, Artificial Sequence
  HC8 leader sequence
MDLLHKNMKHLWFFLLLVAAPRWVLS

SEQ ID NO: 22 - protein, 22 aa, Artificial Sequence
  LC1 leader sequence
MDMRVPAQLLGLLLLWLSGARC

SEQ ID NO: 23 - protein, 22 aa, Artificial Sequence
  LC2 leader sequence
MKYLLPTAAAGLLLLAAQPAMA

SEQ ID NO: 24 - protein, 18 aa, Artificial Sequence
  human chymotrypsinogen B2 signal peptide
MAFLWLLSCWALLGTTFG

SEQ ID NO: 25 - protein, 19 aa, Artificial Sequence
  Gaussia leader sequence
MGVKVLFALICIAVAEAKP

SEQ ID NO: 26 - DNA, 87 bp, Artificial Sequence
  Native mouse MOG leader sequence
atggcatgtttatggtcattttcatggccatcatgttttttatcattattattattattattattacaattatcatgttcatatgca

SEQ ID NO: 27 - DNA, 57 bp, Artificial Sequence
  HC1 leader sequence
atggagttgggactgagctggattttccttttggctattttaaaaggtgtccagtgt

SEQ ID NO: 28 - DNA, 57 bp, Artificial Sequence
  HC2 leader sequence
atggaactggggctccgctgggttttccttgttgctattttagaaggtgtccagtgt

SEQ ID NO: 29 - DNA, 57 bp, Artificial Sequence
  HC3 leader sequence
atgaaacacctgtggttcttcctcctgctggtggcagctcccagatgggtcctgtcc

SEQ ID NO: 30 - DNA, 57 bp, Artificial Sequence
  HC4 leader sequence
atggactggacctggaggatcctcttcttggtggcagcagcaacaggtgcccactcg

SEQ ID NO: 31 - DNA, 57 bp, Artificial Sequence
  HC5 leader sequence
atggactggacctggaggttcctctttgtggtggcagcagctacaggtgtccagtcc

SEQ ID NO: 32 - DNA, 57 bp, Artificial Sequence
  HC6 leader sequence
atggagtttgggctgagctggctttttcttgtggcgattctaaaaggtgtccagtgt

SEQ ID NO: 33 - DNA, 57 bp, Artificial Sequence
  HC7 leader sequence
atggagtttgggctgagctgggttttcctcgttgctctttttagaggtgtccagtgt

SEQ ID NO: 34 - DNA, 78 bp, Artificial Sequence
  HC8 leader sequence
atggacctcctgcacaagaacatgaaacacctgtggttcttcctcctcctggtggcagctcccagatgggtgctgtcc

SEQ ID NO: 35 - DNA, 66 bp, Artificial Sequence
  LC1 leader sequence
atggacatgagggtccctgctcagctcctggggctcctgctgctctggctctcaggtgccagatgt

SEQ ID NO: 36 - DNA, 66 bp, Artificial Sequence
  LC2 leader sequence
atgaaatacctattgcctacggcagccgctggattgttattactcgcggcccagccggccatggcc

SEQ ID NO: 37 - DNA, 51 bp, Artificial Sequence
  human chymotrypsinogen B2 signal peptide
gcctttctgtggctgctgtcctgctgggccctgctggggaccacctttggc

SEQ ID NO: 38 - DNA, 57 bp, Artificial Sequence
  Gaussia leader sequence
atgggagtcaaagttctgtttgccctgatctgcatcgctgtggccgaggccaagccc

SEQ ID NO: 39 - protein, 277 aa, Mus musculus
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 40 - protein, 195 aa, Mus musculus
MGNHSGKRELSAEKASKDGEIHRGEAGKKRSVGKLSQTASEDSDVFGEADAIQNNGTSAEDTAVTDSKHTADPKNNWQGAHPADPGNRPHLIRLFSRDAPGREDNTFKDRPSESDELQTIQEDPTAASGGLDVMASQKRPSQRSKYLATASTMDHARHGFLPRHRDTGILDSIGRFFSGDRGAPKRGSGKVSSEP

SEQ ID NO: 41 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 42 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 43 - protein, 186 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 44 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 45 - protein, 160 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 46 - protein, 197 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKVSSEE

SEQ ID NO: 47 - protein, 277 aa, Homo sapiens; identical to SEQ ID NO: 39

SEQ ID NO: 48 - protein, 242 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 49 - protein, 277 aa, Homo sapiens; identical to SEQ ID NO: 39

SEQ ID NO: 50 - protein, 222 aa, Homo sapiens
MDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 51 - protein, 15 aa, Artificial Sequence
  MBP 30-44
PRHRDTGILDSIGRF

SEQ ID NO: 52 - protein, 17 aa, Artificial Sequence
  MBP 83-99
ENPVVHFFKNIVTPRTP

SEQ ID NO: 53 - protein, 15 aa, Artificial Sequence
  MBP 131-145
ASDYKSAHKGFKGVD

SEQ ID NO: 54 - protein, 15 aa, Artificial Sequence
  MBP 140-154
GFKGVDAQGTLSKIF

SEQ ID NO: 55 - protein, 15 aa, Artificial Sequence
  IRBP 1177-1191
ADGSSWEGVGVVPDV

SEQ ID NO: 56 - protein, 21 aa, Artificial Sequence
  RNB-4K-GKK
KKGNLPNISRIYVSIDVTGKK

SEQ ID NO: 57 - protein, 21 aa, Artificial Sequence
  RNB-5D-K1
KKKKYVSIDVTLQQLESHKKK

SEQ ID NO: 58 - protein, 15 aa, Artificial Sequence
  RNB-9B
GLKMFPDLTKVYSTD

SEQ ID NO: 59 - protein, 15 aa, Artificial Sequence
  S-Arrestin Derived Peptide 1
IFKKISRDKSVTIYL

SEQ ID NO: 60 - protein, 15 aa, Artificial Sequence
  S-Arrestin Derived Peptide 2
KGKKVYVTLTCAFRY

SEQ ID NO: 61 - protein, 22 aa, Artificial Sequence
  S-Arrestin Derived Peptide 3
VIGLTFRRDLYFSRVQVYPPVG

SEQ ID NO: 62 - protein, 37 aa, Artificial Sequence
  S-Arrestin Derived Peptide 4
ESLLKKLGSNTYPFLLTFPDYLPCSVMLQPAPQDSGK

SEQ ID NO: 63 - protein, 17 aa, Artificial Sequence
  S-Arrestin Derived Peptide 5
SSVRLLIRKVQHAPLEM

SEQ ID NO: 64 - protein, 26 aa, Artificial Sequence
  S-Arrestin Derived Peptide 6
AEAAWQFFMSDKPLHLAVSLNKEIYF

SEQ ID NO: 65 - protein, 41 aa, Artificial Sequence
  S-Arrestin Derived Peptide 7
LTKTLTLLPLLANNRERRGIALDGKIKHEDTNLASSTIIKE

SEQ ID NO: 66 - protein, 21 aa, Artificial Sequence
  S-Arrestin Derived Peptide 8
IDRTVLGILVSYQIKVKLTVS

SEQ ID NO: 67 - protein, 29 aa, Artificial Sequence
  S-Arrestin Derived Peptide 9
NTEKTVKKIKAFVEQVANVVLYSSDYYVK

SEQ ID NO: 68 - protein, 42 aa, Artificial Sequence
  HIP-115
MAASGKTSKSEPNHVIFKKISRDKSVTIYLGNRDYIDHVSQV

SEQ ID NO: 69 - protein, 19 aa, Artificial Sequence
  HIP-115NE
HVIFKKISRDKSVTIYLGN

SEQ ID NO: 70 - protein, 11 aa, Artificial Sequence
  HIP-115NE1
KKISRDKSVTI

SEQ ID NO: 71 - protein, 13 aa, Artificial Sequence
  HIP-115NE2
FKKISRDKSVTIY

SEQ ID NO: 72 - protein, 17 aa, Artificial Sequence
  HIP-115NE3
VIFKKISRDKSVTIYLG

SEQ ID NO: 73 - protein, 23 aa, Artificial Sequence
  HIP-115NE1-KKK
KKKVIFKKISRDKSVTIYLGKKK

SEQ ID NO: 74 - protein, 38 aa, Artificial Sequence
  HIP-241125
LTLLPLLANNRERRGIALDGKIKHEDTNLASSTIIKEG

SEQ ID NO: 75 - protein, 17 aa, Artificial Sequence
  HIP-11AC
NRERRGIALDGKIKHED

SEQ ID NO: 76 - protein, 13 aa, Artificial Sequence
  HIP-11AC1
ERRGIALDGKIKH

SEQ ID NO: 77 - protein, 15 aa, Artificial Sequence
  HIP-11B
RERRGIALDGKIKHE

SEQ ID NO: 78 - protein, 20 aa, Artificial Sequence
  HIP-11JO
DGKIKHEDTNLASSTIIKEG

SEQ ID NO: 79 - protein, 10 aa, Artificial Sequence
  HIP-11JO1
HEDTNLASST

SEQ ID NO: 80 - protein, 12 aa, Artificial Sequence
  HIP-11JO2
KHEDTNLASSTI

SEQ ID NO: 81 - protein, 14 aa, Artificial Sequence
  HIP-11JO3
IKHEDTNLASSTII

SEQ ID NO: 82 - protein, 16 aa, Artificial Sequence
  HIP-11JO4
KIKHEDTNLASSTIIK

SEQ ID NO: 83 - protein, 18 aa, Artificial Sequence
  HIP-24DG
LTKTLTLLPLLANNRERR

SEQ ID NO: 84 - protein, 20 aa, Artificial Sequence
  HIP-24HM
LTLLPLLANNRERRGIALDG

SEQ ID NO: 85 - protein, 10 aa, Artificial Sequence
  HIP-24HM1
LLANNRERRG

SEQ ID NO: 86 - protein, 12 aa, Artificial Sequence
  HIP-24HM2
PLLANNRERRGI

SEQ ID NO: 87 - protein, 14 aa, Artificial Sequence
  HIP-24HM3
LPLLANNRERRGIA

SEQ ID NO: 88 - protein, 15 aa, Artificial Sequence
  HIP-24P
NNRERRGIALDGKIK

SEQ ID NO: 89 - protein, 21 aa, Artificial Sequence
  HIP-9FL
VKKIKAFVEQVANVVLYSSDY

SEQ ID NO: 90 - protein, 27 aa, Artificial Sequence
  HIP-9FL-KKK
KKKVKKIKAFVEQVANVVLYSSDYKKK

SEQ ID NO: 91 - protein, 11 aa, Artificial Sequence
  HIP-9K1
AFVEQVANVVL

SEQ ID NO: 92 - protein, 17 aa, Artificial Sequence
  HIP-9K1-KKK
KKKAFVEQVANVVLKKK

SEQ ID NO: 93 - protein, 22 aa, Artificial Sequence
  HIP-17GN
VIGLTFRRDLYFSRVQVYPPVG

SEQ ID NO: 94 - protein, 28 aa, Artificial Sequence
  HIP-17GN-KKK
KKKVIGLTFRRDLYFSRVQVYPPVGKKK

SEQ ID NO: 95 - protein, 15 aa, Artificial Sequence
  HIP-17J
LTFRRDLYFSRVQVY

SEQ ID NO: 96 - protein, 21 aa, Artificial Sequence
  HIP-17J-KKK
KKKLTFRRDLYFSRVQVYKKK

SEQ ID NO: 97 - protein, 12 aa, Artificial Sequence
  HIP-17K1
TFRRDLYFSRVQ

SEQ ID NO: 98 - protein, 10 aa, Artificial Sequence
  HIP-17M1
RRDLYFSRVQ

SEQ ID NO: 99 - protein, 25 aa, Artificial Sequence
  HIP-12AK
IDRTVLGILVSYQIKVKLTVSGFLG

SEQ ID NO: 100 - protein, 31 aa, Artificial Sequence
  HIP-12AK-KKK
KKKIDRTVLGILVSYQIKVKLTVSGFLGKKK

SEQ ID NO: 101 - protein, 15 aa, Artificial Sequence
  HIP-12E
VLGILVSYQIKVKLT

SEQ ID NO: 102 - protein, 21 aa, Artificial Sequence
  HIP-12E-KKK
KKKVLGILVSYQIKVKLTKKK

SEQ ID NO: 103 - protein, 11 aa, Artificial Sequence
  HIP-12G1
GILVSYQIKVK

SEQ ID NO: 104 - protein, 17 aa, Artificial Sequence
  HIP-12G1-KKK
KKKGILVSYQIKVKKKK

SEQ ID NO: 105 - protein, 15 aa, Artificial Sequence
  Myosin Derived Peptide 1
MLTFLHEPAVLFNLK

SEQ ID NO: 106 - protein, 14 aa, Artificial Sequence
  Myosin Derived Peptide 2
VNPYKWLPVYNAEW

SEQ ID NO: 107 - protein, 15 aa, Artificial Sequence
  Myosin Derived Peptide 3
PHIFSISDNAYQYML

SEQ ID NO: 108 - protein, 15 aa, Artificial Sequence
  Myosin Derived Peptide 4
KRVIQYFASIAAIGD

SEQ ID NO: 109 - protein, 15 aa, Artificial Sequence
  Myosin Derived Peptide 5
YHIFYQILSNKKPEL

SEQ ID NO: 110 - protein, 15 aa, Artificial Sequence
  Myosin Derived Peptide 6
KSAYLMGLNSADLLK

SEQ ID NO: 111 - protein, 15 aa, Artificial Sequence
  Myosin Derived Peptide 7
KSSLKLMATLFSSYA

SEQ ID NO: 112 - protein, 15 aa, Artificial Sequence
  Myosin Derived Peptide 8
KGSSFQTVSALHREN

SEQ ID NO: 113 - protein, 15 aa, Artificial Sequence
  Myosin Derived Peptide 9
EATLQHEATAAALRK

SEQ ID NO: 114 - protein, 15 aa, Artificial Sequence
  Myosin Derived Peptide 10
RVQLLHSQNTSLINQ

SEQ ID NO: 115 - protein, 29 aa, Artificial Sequence
  RNB 5 (1)
ISRIYVSIDVTLQQLESHSFYNLSKVTHI

SEQ ID NO: 116 - protein, 29 aa, Artificial Sequence
  RNB 4 (2)
LRTIPSHAFSNLPNISRIYVSIDVTLQQL

SEQ ID NO: 117 - protein, 15 aa, Artificial Sequence
  RNB 4A
LRTIPSHAFSNLPNI

SEQ ID NO: 118 - protein, 15 aa, Artificial Sequence
  RNB 4B
RTIPSHAFSNLPNIS

SEQ ID NO: 119 - protein, 15 aa, Artificial Sequence
  RNB 4C
TIPSHAFSNLPNISR

SEQ ID NO: 120 - protein, 15 aa, Artificial Sequence
  RNB 4D
IPSHAFSNLPNISRI

SEQ ID NO: 121 - protein, 15 aa, Artificial Sequence
  RNB 4E
PSHAFSNLPNISRIY

SEQ ID NO: 122 - protein, 16 aa, Artificial Sequence
  RNB 4F
  misc_feature (1)..(1): Xaa can be any naturally occurring amino acid
XSHAFSNLPNISRIYV

SEQ ID NO: 123 - protein, 15 aa, Artificial Sequence
  RNB 4G
HAFSNLPNISRIYVS

SEQ ID NO: 124 - protein, 15 aa, Artificial Sequence
  RNB 4H
AFSNLPNISRIYVSI

SEQ ID NO: 125 - protein, 15 aa, Artificial Sequence
  RNB 4I
FSNLPNISRIYVSID

SEQ ID NO: 126 - protein, 15 aa, Artificial Sequence
  RNB 4J
SNLPNISRIYVSIDV

SEQ ID NO: 127 - protein, 15 aa, Artificial Sequence
  RNB 4K
NLPNISRIYVSIDVT

SEQ ID NO: 128 - protein, 15 aa, Artificial Sequence
  RNB 4L
LPNISRIYVSIDVTL

SEQ ID NO: 129 - protein, 15 aa, Artificial Sequence
  RNB 4M
PNISRIYVSIDVTLQ

SEQ ID NO: 130 - protein, 15 aa, Artificial Sequence
  RNB 4N
NISRIYVSIDVTLQQ

SEQ ID NO: 131 - protein, 21 aa, Artificial Sequence
  RNB 4J-GKK
KKGSNLPNISRIYVSIDVGKK

SEQ ID NO: 132 - protein, 21 aa, Artificial Sequence
  RNB 4K-GKK
KKGNLPNISRIYVSIDVTGKK

SEQ ID NO: 133 - protein, 29 aa, Artificial Sequence
  RNB_9 (3)
TGLKMFPDLTKVYSTDIFFILEITDNPYM

SEQ ID NO: 134 - protein, 15 aa, Artificial Sequence
  RNB 9A
TGLKMFPDLTKVYST

SEQ ID NO: 135 - protein, 15 aa, Artificial Sequence
  RNB 9B
GLKMFPDLTKVYSTD

SEQ ID NO: 136 - protein, 15 aa, Artificial Sequence
  RNB 9C
LKMFPDLTKVYSTDI

SEQ ID NO: 137 - protein, 15 aa, Artificial Sequence
  RNB 9D
KMFPDLTKVYSTDTF

SEQ ID NO: 138 - protein, 15 aa, Artificial Sequence
  RNB_5A (6)
ISRIYVSIDVTLQQL

SEQ ID NO: 139 - protein, 15 aa, Artificial Sequence
  RNB_5B (7)
SRIYVSIDVTLQQLE

SEQ ID NO: 140 - protein, 15 aa, Artificial Sequence
  RNB 5C (8)
RIYVSIDVTLQQLES

SEQ ID NO: 141 - protein, 15 aa, Artificial Sequence
  RNB 5D (9)
IYVSIDVTLQQLESH

SEQ ID NO: 142 - protein, 15 aa, Artificial Sequence
  RNB 5E (10)
YVSIDVTLQQLESHS

SEQ ID NO: 143 - protein, 15 aa, Artificial Sequence
  RNB 5F (11)
VSIDVTLQQLESHSF

SEQ ID NO: 144 - protein, 15 aa, Artificial Sequence
  RNB 5G
SIDVTLQQLESHSFY

SEQ ID NO: 145 - protein, 15 aa, Artificial Sequence
  RNB 5H
IDVTLQQLESHSFYN

SEQ ID NO: 146 - protein, 15 aa, Artificial Sequence
  RNB 5I
DVTLQQLESHSFYNL

SEQ ID NO: 147 - protein, 21 aa, Artificial Sequence
  RNB 5D-GKK (12)
KKGIYVSIDVTLQQLESHGKK

SEQ ID NO: 148 - protein, 21 aa, Artificial Sequence
  RNB_5E-GKK (13)
KKGYVSIDVTLQQLESHSGKK

SEQ ID NO: 149 - protein, 21 aa, Artificial Sequence
  RNB 5F-GKK (14)
KKGVSIDVTLQQLESHSFGKK

SEQ ID NO: 150 - protein, 15 aa, Artificial Sequence
  RNB 5J
VTLQQLESHSFYNLS

SEQ ID NO: 151 - protein, 15 aa, Artificial Sequence
  RNB 5K
TLQQLESHSFYNLSK

SEQ ID NO: 152 - protein, 15 aa, Artificial Sequence
  RNB 5L
LQQLESHSFYNLSKV

SEQ ID NO: 153 - protein, 15 aa, Artificial Sequence
  RNB 5M
QQLESHSFYNLSKVT

SEQ ID NO: 154 - protein, 15 aa, Artificial Sequence
  RNB 5N
QLESHSFYNLSKVTH

SEQ ID NO: 155 - protein, 15 aa, Artificial Sequence
  RNB 5O
LESHSFYNLSKVTHI

SEQ ID NO: 156 - protein, 15 aa, Artificial Sequence
  RNB_4J
SNLPNISRIYVSIDV

SEQ ID NO: 157 - protein, 21 aa, Artificial Sequence
  RNB 4J-GKK
KKGSNLPNISRIYVSIDVGKK

SEQ ID NO: 158 - protein, 15 aa, Artificial Sequence
  RNB 9A (17)
TGLKMFPDLTKVYST

SEQ ID NO: 159 - protein, 15 aa, Artificial Sequence
  RNB 9B (18)
GLKMFPDLTKVYSTD

SEQ ID NO: 160 - protein, 15 aa, Artificial Sequence
  RNB_9C (19)
LKMFPDLTKVYSTDI

SEQ ID NO: 161 - protein, 15 aa, Artificial Sequence
  RNB 9D (20)
KMFPDLTKVYSTDIF

SEQ ID NO: 162 - protein, 21 aa, Artificial Sequence
  Thyroid Stimulating Hormone Receptor Derived Peptide (21
KKKIYVSIDVTLQQLESHKKK

SEQ ID NO: 163 - protein, 21 aa, Artificial Sequence
  Thyroid Stimulating Hormone Receptor Derived Peptide (22)
KKGKYVSIDVTLQQLESHGKK

SEQ ID NO: 164 - protein, 21 aa, Artificial Sequence
  Thyroid Stimulating Hormone Receptor Derived Peptide (23)
KKGIKVSIDVTLQQLESHGKK

SEQ ID NO: 165 - protein, 21 aa, Artificial Sequence
  Thyroid Stimulating Hormone Receptor Derived Peptide (24)
KKGIYKSIDVTLQQLESHGKK

SEQ ID NO: 166 - protein, 21 aa, Artificial Sequence
  Thyroid Stimulating Hormone Receptor Derived Peptide (25)
KKGIYVSIDVKLQQLESHGKK

SEQ ID NO: 167 - protein, 21 aa, Artificial Sequence
  Thyroid Stimulating Hormone Receptor Derived Peptide (26)
KKGIYVSIDVTLQKLESHGKK

SEQ ID NO: 168 - protein, 21 aa, Artificial Sequence
  Thyroid Stimulating Hormone Receptor Derived Peptide (27)
KKGIYVSIDVTLQQKESHGKK

SEQ ID NO: 169 - protein, 21 aa, Artificial Sequence
  Thyroid Stimulating Hormone Receptor Derived Peptide (28)
KKGIYVSIDVTLQQLKSHGKK

SEQ ID NO: 170 - protein, 21 aa, Artificial Sequence
  Thyroid Stimulating Hormone Receptor Derived Peptide (29)
KKGIYVSIDVTLQQLEKHGKK

SEQ ID NO: 171 - protein, 21 aa, Artificial Sequence
  Thyroid Stimulating Hormone Receptor Derived Peptide (30)
KKGIYVSIDVTLQQLESKGKK

SEQ ID NO: 172 - protein, 18 aa, Artificial Sequence
  Thyroid Stimulating Hormone Receptor Derived Peptide (31)
KKGYVSIDVTLQQLEGKK

SEQ ID NO: 173 - protein, 18 aa, Artificial Sequence
  Thyroid Stimulating Hormone Receptor Derived Peptide (32)
KKGYVSIDVKLQQLEGKK

SEQ ID NO: 174 - protein, 18 aa, Artificial Sequence
  Thyroid Stimulating Hormone Receptor Derived Peptide (33)
KKGYVSIDVTLQKLEGKK

SEQ ID NO: 175 - protein, 18 aa, Artificial Sequence
  Thyroid Stimulating Hormone Receptor Derived Peptide (34)
KKGYVSIDVTLQQKEGKK

SEQ ID NO: 176 - protein, 18 aa, Artificial Sequence
  Thyroid Stimulating Hormone Receptor Derived Peptide (35)
KKGYVSIDVKLQKKEGKK

SEQ ID NO: 177 - protein, 19 aa, Artificial Sequence
  Thyroid Stimulating Hormone Receptor Derived Peptide (36)
KKGIYVSIDVTLQQLEGKK

SEQ ID NO: 178 - protein, 19 aa, Artificial Sequence
  Thyroid Stimulating Hormone Receptor Derived Peptide (37)
KKGIYVSIDVKLQQLEGKK

SEQ ID NO: 179 - protein, 19 aa, Artificial Sequence
  Thyroid Stimulating Hormone Receptor Derived Peptide (38)
KKGIYVSIDVTLQKLEGKK

SEQ ID NO: 180 - protein, 19 aa, Artificial Sequence
  Thyroid Stimulating Hormone Receptor Derived Peptide (39)
KKGIYVSIDVTLQQKEGKK

SEQ ID NO: 181 - protein, 19 aa, Artificial Sequence
  Thyroid Stimulating Hormone Receptor Derived Peptide (40)
KKGIYVSIDVKLQKKEGKK

SEQ ID NO: 182 - protein, 19 aa, Artificial Sequence
  Thyroid Stimulating Hormone Receptor Derived Peptide (41)
KKGTYVSIDVTLQQLEGKK

SEQ ID NO: 183 - protein, 19 aa, Artificial Sequence
  Thyroid Stimulating Hormone Receptor Derived Peptide (42)
KKGTYVSIDVKLQQLEGKK

SEQ ID NO: 184 - protein, 19 aa, Artificial Sequence
  Thyroid Stimulating Hormone Receptor Derived Peptide (43)
KKGTYVSIDVTLQKLEGKK

SEQ ID NO: 185 - protein, 19 aa, Artificial Sequence
  Thyroid Stimulating Hormone Receptor Derived Peptide (44)
KKGTYVSIDVTLQQKEGKK

SEQ ID NO: 186 - protein, 19 aa, Artificial Sequence
  Thyroid Stimulating Hormone Receptor Derived Peptide (45)
KKGTYVSIDVKLQKKEGKK

SEQ ID NO: 187 - protein, 21 aa, Artificial Sequence
  Thyroid Stimulating Hormone Receptor Derived Peptide (46)
KKKKYVSIDVTLQQLESHKKK

SEQ ID NO: 188 - protein, 21 aa, Artificial Sequence
  Thyroid Stimulating Hormone Receptor Derived Peptide (47)
KKKIKVSIDVTLQQLESHKKK

SEQ ID NO: 189 - protein, 21 aa, Artificial Sequence
  Thyroid Stimulating Hormone Receptor Derived Peptide (48)
KKKIYKSIDVTLQQLESHKKK

SEQ ID NO: 190 - protein, 21 aa, Artificial Sequence
  Thyroid Stimulating Hormone Receptor Derived Peptide (49)
KKKIYVKIDVTLQQLESHKKK

SEQ ID NO: 191 - protein, 21 aa, Artificial Sequence
  Thyroid Stimulating Hormone Receptor Derived Peptide (50)
KKKIYVSIDVKLQQLESHKKK

SEQ ID NO: 192 - protein, 21 aa, Artificial Sequence
  Thyroid Stimulating Hormone Receptor Derived Peptide (51)
KKKIYVSIDVTLKQLESHKKK

SEQ ID NO: 193 - protein, 21 aa, Artificial Sequence
  Thyroid Stimulating Hormone Receptor Derived Peptide (52)
KKKIYVSIDVTLQKLESHKKK

SEQ ID NO: 194 - protein, 21 aa, Artificial Sequence
  Thyroid Stimulating Hormone Receptor Derived Peptide (53)
KKKIYVSIDVTLQQKESHKKK

SEQ ID NO: 195 - protein, 21 aa, Artificial Sequence
  Thyroid Stimulating Hormone Receptor Derived Peptide (54)
KKKIYVSIDVTLQQLKSHKKK

SEQ ID NO: 196 - protein, 21 aa, Artificial Sequence
  Thyroid Stimulating Hormone Receptor Derived Peptide (55)
KKKIYVSIDVTLQQLEKHKKK

SEQ ID NO: 197 - protein, 21 aa, Artificial Sequence
  Thyroid Stimulating Hormone Receptor Derived Peptide (56)
KKKIYVSIDVTLQQLESKKKK

SEQ ID NO: 198 - protein, 18 aa, Artificial Sequence
  Thyroid Stimulating Hormone Receptor Derived Peptide (57)
KKKYVSIDVTLQQLEKKK

SEQ ID NO: 199 - protein, 18 aa, Artificial Sequence
  Thyroid Stimulating Hormone Receptor Derived Peptide (58)
KKKYVSIDVKLQQLEKKK

SEQ ID NO: 200 - protein, 18 aa, Artificial Sequence
  Thyroid Stimulating Hormone Receptor Derived Peptide (59)
KKKYVSIDVTLQKLEKKK

SEQ ID NO: 201 - protein, 18 aa, Artificial Sequence
  Thyroid Stimulating Hormone Receptor Derived Peptide (60)
KKKYVSIDVTLQQKEKKK

SEQ ID NO: 202 - protein, 18 aa, Artificial Sequence
  Thyroid Stimulating Hormone Receptor Derived Peptide (61)
KKKYVSIDVKLQKKEKKK

SEQ ID NO: 203 - protein, 15 aa, Artificial Sequence
  Thyroid Stimulating Hormone Receptor Derived Peptide (62)
NLPNISRIYVSIDVT

SEQ ID NO: 204 - protein, 21 aa, Artificial Sequence
  Thyroid Stimulating Hormone Receptor Derived Peptide (63)
KKGNLPNISRIYVSIDVTGKK

SEQ ID NO: 205 - protein, 28 aa, Artificial Sequence
  Thyroid Stimulating Hormone Receptor Derived Peptide (64)
LTLKLYNNGFTSVQGYAFNGTKLDAVYL

SEQ ID NO: 206 - protein, 16 aa, Artificial Sequence
  Thyroid Stimulating Hormone Receptor Derived Peptide (65)
LTLKLYNNGFTSVQGY

SEQ ID NO: 207 - protein, 15 aa, Artificial Sequence
  Thyroid Stimulating Hormone Receptor Derived Peptide (66)
TLKLYNNGFTSVQGY

SEQ ID NO: 208 - protein, 21 aa, Artificial Sequence
  Thyroid Stimulating Hormone Receptor Derived Peptide (67)
KKKTLKLYNNGFTSVQGYKKK

SEQ ID NO: 209 - protein, 15 aa, Artificial Sequence
  Thyroid Stimulating Hormone Receptor Derived Peptide (68)
LKLYNNGFTSVQGYA

SEQ ID NO: 210 - protein, 15 aa, Artificial Sequence
  Thyroid Stimulating Hormone Receptor Derived Peptide (69)
KLYNNGFTSVQGYAF

SEQ ID NO: 211 - protein, 15 aa, Artificial Sequence
  Thyroid Stimulating Hormone Receptor Derived Peptide (70)
LYNNGFTSVQGYAFN

SEQ ID NO: 212 - protein, 15 aa, Artificial Sequence
  Thyroid Stimulating Hormone Receptor Derived Peptide (71)
YNNGFTSVQGYAFNG

SEQ ID NO: 213 - protein, 15 aa, Artificial Sequence
  Thyroid Stimulating Hormone Receptor Derived Peptide (72)
NNGFTSVQGYAFNGT

SEQ ID NO: 214 - protein, 15 aa, Artificial Sequence
  Thyroid Stimulating Hormone Receptor Derived Peptide (73)
NGFTSVQGYAFNGTK

SEQ ID NO: 215 - protein, 15 aa, Artificial Sequence
  Thyroid Stimulating Hormone Receptor Derived Peptide (74)
GFTSVQGYAFNGTKL

SEQ ID NO: 216 - protein, 15 aa, Artificial Sequence
  Thyroid Stimulating Hormone Receptor Derived Peptide (75)
FTSVQGYAFNGTKLD

SEQ ID NO: 217 - protein, 15 aa, Artificial Sequence
  Thyroid Stimulating Hormone Receptor Derived Peptide (76)
TSVQGYAFNGTKLDA

SEQ ID NO: 218 - protein, 15 aa, Artificial Sequence
  Thyroid Stimulating Hormone Receptor Derived Peptide (77)
SVQGYAFNGTKLDAV

SEQ ID NO: 219 - protein, 15 aa, Artificial Sequence
  Thyroid Stimulating Hormone Receptor Derived Peptide (78)
VQGYAFNGTKLDAVY

SEQ ID NO: 220 - protein, 29 aa, Artificial Sequence
  RNB 1
GGMGCSSPPCECHQEEDFRVTCKDIQRIP

SEQ ID NO: 221 - protein, 30 aa, Artificial Sequence
  RNB 2
EEDFRVTCKDIQRIPSLPPSTQTLKLIETH

SEQ ID NO: 222 - protein, 30 aa, Artificial Sequence
  RNB 3
SLPPSTQTLKLIETHLRTIPSHAFSNLPNI

SEQ ID NO: 223 - protein, 29 aa, Artificial Sequence
  RNB 6
ESHSFYNLSKVTHIEIRNTRNLTYIDPDA

SEQ ID NO: 224 - protein, 29 aa, Artificial Sequence
  RNB 7
EIRNTRNLTYIDPDALKELPLLKFLGIFN

SEQ ID NO: 225 - protein, 29 aa, Artificial Sequence
  RNB 8
LKELPLLKFLGIFNTGLKMFPDLTKVYST

SEQ ID NO: 226 - protein, 29 aa, Artificial Sequence
  RNB 9
TGLKMFPDLTKVYSTDIFFILEITDNPYM

SEQ ID NO: 227 - protein, 30 aa, Artificial Sequence
  RNB 10
TDIFFILEITDNPYMTSIPVNAFQGLCNET

SEQ ID NO: 228 - protein, 28 aa, Artificial Sequence
  RNB 11
TSIPVNAFQGLCNETLTLKLYNNGFTSV

SEQ ID NO: 229 - protein, 28 aa, Artificial Sequence
  RNB 12
LTLKLYNNGFTSVQGYAFNGTKLDAVYL

SEQ ID NO: 230 - protein, 28 aa, Artificial Sequence
  RNB 13
QGYAFNGTKLDAVYLNKNKYLTVIDKDA

SEQ ID NO: 231 - protein, 28 aa, Artificial Sequence
  RNB 14
NKNKYLTVIDKDAFGGVYSGPSLLDVSQ

SEQ ID NO: 232 - protein, 28 aa, Artificial Sequence
  RNB 15
FGGVYSGPSLLDVSQTSVTALPSKGLEH

SEQ ID NO: 233 - protein, 28 aa, Artificial Sequence
  RNB 16
TSVTALPSKGLEHLKELIARNTWTLKKL

SEQ ID NO: 234 - protein, 28 aa, Artificial Sequence
  RNB 17
LKELIARNTWTLKKLPLSLSFLHLTRAD

SEQ ID NO: 235 - protein, 28 aa, Artificial Sequence
  RNB 18
PLSLSFLHLTRADLSYPSHCCAFKNQKK

SEQ ID NO: 236 - protein, 28 aa, Artificial Sequence
  RNB 19
LSYPSHCCAFKNQKKIRGILESLMCNES

SEQ ID NO: 237 - protein, 28 aa, Artificial Sequence
  RNB 20
IRGILESLMCNESSMQSLRQRKSVNALN

SEQ ID NO: 238 - protein, 29 aa, Artificial Sequence
  RNB 21
SMQSLRQRKSVNALNSPLHQEYEENLGDS

SEQ ID NO: 239 - protein, 29 aa, Artificial Sequence
  RNB 22
SPLHQEYEENLGDSIVGYKEKSKFQDTHN

SEQ ID NO: 240 - protein, 29 aa, Artificial Sequence
  RNB 23
IVGYKEKSKFQDTHNNAHYYVFFEEQEDE

SEQ ID NO: 241 - protein, 29 aa, Artificial Sequence
  RNB 24
NAHYYVFFEEQEDEIIGFGQELKNPQEET

SEQ ID NO: 242 - protein, 29 aa, Artificial Sequence
  RNB 25
IIGFGQELKNPQEETLQAFDSHYDYTICG

SEQ ID NO: 243 - protein, 29 aa, Artificial Sequence
  RNB 26
LQAFDSHYDYTICGDSEDMVCTPKSDEFN

SEQ ID NO: 244 - protein, 27 aa, Artificial Sequence
  RNB 27
DSEDMVCTPKSDEFNPCEDIMGYKFLR

SEQ ID NO: 245 - protein, 29 aa, Artificial Sequence
  RNB 28
KLDAVYLNKNKYLTVIDKDAFGGVYSGPS

SEQ ID NO: 246 - protein, 15 aa, Artificial Sequence
  MOG 41-55
RPPFSRVVHLYRNGK

SEQ ID NO: 247 - protein, 15 aa, Artificial Sequence
  MOG 43-57
PFSRVVHLYRNGKDQ

SEQ ID NO: 248 - protein, 15 aa, Artificial Sequence
  MOG 44-58
FSRVVHLYRNGKDQD

SEQ ID NO: 249 - protein, 15 aa, Artificial Sequence
  MOG 45-59
SRVVHLYRNGKDQDG

SEQ ID NO: 250 - protein, 21 aa, Artificial Sequence
  MOG 35-55
MEVGWYRSPFSRVVHLYRNGK

SEQ ID NO: 251 - protein, 21 aa, Artificial Sequence
  MOG 40-60
YRPPFSRVVHLYRNGKDQDGD

SEQ ID NO: 252 - protein, 26 aa, Artificial Sequence
  PLP 36-61
HEALTGTEKLIETYFSKNYQDYEYLI

SEQ ID NO: 253 - protein, 28 aa, Artificial Sequence
  PLP 179-206
TWTTCQSIAFPSKTSASIGSLCADARMY

SEQ ID NO: 254 - protein, 28 aa, Artificial Sequence
  PLP 207-234
GVLPWNAFPGKVCGSNLLSICKTAEFQM

SEQ ID NO: 255 - protein, 19 aa, Artificial Sequence
  PLP 39-57
LTGTEKLIETYFSKNYQDY

SEQ ID NO: 256 - protein, 19 aa, Artificial Sequence
  PLP 180-198
WTTCQSIAFPSKTSASIGS

SEQ ID NO: 257 - protein, 15 aa, Artificial Sequence
  PLP 208-222
VLPWNAFPGKVCGSN

SEQ ID NO: 258 - protein, 15 aa, Artificial Sequence
  PLP 39-53
LTGTEKLIETYFSKN

SEQ ID NO: 259 - protein, 15 aa, Artificial Sequence
  PLP 42-56
TEKLIETYFSKNYQD

SEQ ID NO: 260 - protein, 15 aa, Artificial Sequence
  PLP 43-57
EKLIETYFSKNYQDY

SEQ ID NO: 261 - protein, 15 aa, Artificial Sequence
  PLP 180-194
WTTCQSIAFPSKTSA

SEQ ID NO: 262 - protein, 15 aa, Artificial Sequence
  PLP 181-195
TTCQSIAFPSKTSAS

SEQ ID NO: 263 - protein, 15 aa, Artificial Sequence
  PLP 182-196
TCQSIAFPSKTSASI

SEQ ID NO: 264 - protein, 15 aa, Artificial Sequence
  PLP183-197
CQSIAFPSKTSASIG

SEQ ID NO: 265 - protein, 15 aa, Artificial Sequence
  PLP 184-198
QSIAFPSKTSASIGS

SEQ ID NO: 266 - protein, 15 aa, Artificial Sequence
  PLP 208-222
VLPWNAFPGKVCGSN

SEQ ID NO: 267 - protein, 15 aa, Artificial Sequence
  POP-1
HEALTGTEKLIETYF

SEQ ID NO: 268 - protein, 15 aa, Artificial Sequence
  POP-2
EALTGTEKLIETYFS

SEQ ID NO: 269 - protein, 15 aa, Artificial Sequence
  POP-3
ALTGTEKLIETYFSK

SEQ ID NO: 270 - protein, 15 aa, Artificial Sequence
  POP-4
LTGTEKLIETYFSKN

SEQ ID NO: 271 - protein, 15 aa, Artificial Sequence
  POP-5
TGTEKLIETYFSKNY

SEQ ID NO: 272 - protein, 15 aa, Artificial Sequence
  POP-6
GTEKLIETYFSKNYQ

SEQ ID NO: 273 - protein, 15 aa, Artificial Sequence
  POP-7
TEKLIETYFSKNYQD

SEQ ID NO: 274 - protein, 15 aa, Artificial Sequence
  POP-8
EKLIETYFSKNYQDY

SEQ ID NO: 275 - protein, 15 aa, Artificial Sequence
  POP-9
KLIETYFSKNYQDYE

SEQ ID NO: 276 - protein, 15 aa, Artificial Sequence
  POP-10
LIETYFSKNYQDYEY

SEQ ID NO: 277 - protein, 15 aa, Artificial Sequence
  POP-11
IETYFSKNYQDYEYL

SEQ ID NO: 278 - protein, 15 aa, Artificial Sequence
  POP-12
ETYFSKNYQDYEYLI

SEQ ID NO: 279 - protein, 15 aa, Artificial Sequence
  POP-14
WTTCQSIAFPSKTSA

SEQ ID NO: 280 - protein, 15 aa, Artificial Sequence
  POP-15
TCQSIAPPSKTSASI

SEQ ID NO: 281 - protein, 15 aa, Artificial Sequence
  POP-16
CQSIAFPSKTSASIG

SEQ ID NO: 282 - protein, 15 aa, Artificial Sequence
  POP-18
QSIAFPSKTSASIGS

SEQ ID NO: 283 - protein, 21 aa, Artificial Sequence
  FVIII Derived Peptide 1
KKGPRCLTRYYSSFVNMEGKK

SEQ ID NO: 284 - protein, 21 aa, Artificial Sequence
  FVIII Derived Peptide 2
KKGPRCLTRYYSSFVNMEGKE

SEQ ID NO: 285 - protein, 21 aa, Artificial Sequence
  FVIII Derived Peptide 3
KKGPRCLTRYYSSFVNMEGEK

SEQ ID NO: 286 - protein, 21 aa, Artificial Sequence
  FVIII Derived Peptide 4
KKGPRCLTRYYSSFVNMEGEE

SEQ ID NO: 287 - protein, 21 aa, Artificial Sequence
  FVIII Derived Peptide 5
EEGPRCLTRYYSSFVNMEGKK

SEQ ID NO: 288 - protein, 21 aa, Artificial Sequence
  FVIII Derived Peptide 6
EEGPRCLTRYYSSFVNMEGKE

SEQ ID NO: 289 - protein, 21 aa, Artificial Sequence
  FVIII Derived Peptide 7
EEGPRCLTRYYSSFVNMEGEK

SEQ ID NO: 290 - protein, 21 aa, Artificial Sequence
  FVIII Derived Peptide 8
EEGPRCLTRYYSSFVNMEGEE

SEQ ID NO: 291 - protein, 21 aa, Artificial Sequence
  FVIII Derived Peptide 9
KEGPRCLTRYYSSFVNMEGKE

SEQ ID NO: 292 - protein, 21 aa, Artificial Sequence
  FVIII Derived Peptide 10
KEGPRCLTRYYSSFVNMEGKE

SEQ ID NO: 293 - protein, 21 aa, Artificial Sequence
  FVIII Derived Peptide 11
KEGPRCLTRYYSSFVNMEGEK

SEQ ID NO: 294 - protein, 21 aa, Artificial Sequence
  FVIII Derived Peptide 12
KEGPRCLTRYYSSFVNMEGEE

SEQ ID NO: 295 - protein, 21 aa, Artificial Sequence
  FVIII Derived Peptide 13
EKGPRCLTRYYSSFVNMEGKK

SEQ ID NO: 296 - protein, 21 aa, Artificial Sequence
  FVIII Derived Peptide 14
EKGPRCLTRYYSSFVNMEGKE

SEQ ID NO: 297 - protein, 21 aa, Artificial Sequence
  FVIII Derived Peptide 15
EKGPRCLTRYYSSFVNMEGEK

SEQ ID NO: 298 - protein, 21 aa, Artificial Sequence
  FVIII Derived Peptide 16
EKGPRCLTRYYSSFVNMEGEE

SEQ ID NO: 299 - protein, 21 aa, Artificial Sequence
  FVIII Derived Peptide 17
KKGDNIMVTFRNQASRPYGKK

SEQ ID NO: 300 - protein, 21 aa, Artificial Sequence
  FVIII Derived Peptide 18
KKGDNIMVTFRNQASRPYGKE

SEQ ID NO: 301 - protein, 21 aa, Artificial Sequence
  FVIII Derived Peptide 19
KKGDNIMVTFRNQASRPYGEK

SEQ ID NO: 302 - protein, 21 aa, Artificial Sequence
  FVIII Derived Peptide 20
KKGDNIMVTFRNQASRPYGEE

SEQ ID NO: 303 - protein, 21 aa, Artificial Sequence
  FVIII Derived Peptide 21
EEGDNIMVTFRNQASRPYGKK

SEQ ID NO: 304 - protein, 21 aa, Artificial Sequence
  FVIII Derived Peptide 22
EEGDNIMVTFRNQASRPYGKE

SEQ ID NO: 305 - protein, 21 aa, Artificial Sequence
  FVIII Derived Peptide 23
EEGDNIMVTFRNQASRPYGEK

SEQ ID NO: 306 - protein, 21 aa, Artificial Sequence
  FVIII Derived Peptide 34
EEGDNIMVTFRNQASRPYGEE

SEQ ID NO: 307 - protein, 21 aa, Artificial Sequence
  FVIII Derived Peptide 25
KEGDNIMVTFRNQASRPYGKK

SEQ ID NO: 308 - protein, 22 aa, Artificial Sequence
  FVIII Derived Peptide 26
KEGDNIMVTFRNSQASRPYGKE

SEQ ID NO: 309 - protein, 21 aa, Artificial Sequence
  FVIII Derived Peptide 27
KEGDNIMVTFRNQASRPYGEK

SEQ ID NO: 310 - protein, 21 aa, Artificial Sequence
  FVIII Derived Peptide 28
KEGDNIMVTFRNQASRPYGEE

SEQ ID NO: 311 - protein, 21 aa, Artificial Sequence
  FVIII Derived Peptide 29
EKGDNIMVTFRNQASRPYGKK

SEQ ID NO: 312 - protein, 21 aa, Artificial Sequence
  FVIII Derived Peptide 30
EKGDNIMVTFRNQASRPYGKE

SEQ ID NO: 313 - protein, 21 aa, Artificial Sequence
  FVIII Derived Peptide 31
EKGDNIMVTFRNQASRPYGEK

SEQ ID NO: 314 - protein, 21 aa, Artificial Sequence
  FVIII Derived Peptide 32
EKGDNIMVTFRNQASRPYGEE

SEQ ID NO: 315 - protein, 15 aa, Artificial Sequence
  FVIII Derived Peptide 34
DNIMVTFRNQASRPY

SEQ ID NO: 316 - protein, 15 aa, Artificial Sequence
  FVIII Derived Peptide 35
PRCLTRYYSSFVNME

SEQ ID NO: 317 - protein, 5 aa, Artificial Sequence
  FVIII Derived Peptide 36
PRCLT

SEQ ID NO: 318 - protein, 5 aa, Artificial Sequence
  FVIII Derived Peptide 37
DNIMV

SEQ ID NO: 319 - protein, 21 aa, Artificial Sequence
  FVIII Derived Peptide 38, X is K or E
  misc_feature (1)..(2): Xaa can be any naturally occurring amino acid
  misc_feature (20)..(21): Xaa can be any naturally occurring amino acid
XXGDNIMVTFRNQASRPYGXX

SEQ ID NO: 320 - protein, 21 aa, Artificial Sequence
  FVIII Derived Peptide 39, X is K or E
  misc_feature (1)..(2): Xaa can be any naturally occurring amino acid
  misc_feature (20)..(21): Xaa can be any naturally occurring amino acid
XXGPRCLTRYYSSFVNMEGXX

SEQ ID NO: 321 - protein, 9 aa, Artificial Sequence
  FVIII Derived Peptide 40
ASQYRPSQR

SEQ ID NO: 322 - protein, 15 aa, Artificial Sequence
  FVIII Derived Peptide 1
GTLMVFFGNVDSSGI

SEQ ID NO: 323 - protein, 15 aa, Artificial Sequence
  FVIII Derived Peptide 2
TQTLHKFILLFAVFD

SEQ ID NO: 324 - protein, 15 aa, Artificial Sequence
  FVIII Derived Peptide 3
SLYISQFIIMYSLDG

SEQ ID NO: 325 - protein, 15 aa, Artificial Sequence
  FVIII Derived Peptide 3.1
RQKFSSLYISQFIIM

SEQ ID NO: 326 - protein, 15 aa, Artificial Sequence
  FVIII Derived Peptide 3.2
QKFSSLYISQFIIMY

SEQ ID NO: 327 - protein, 15 aa, Artificial Sequence
  FVIII Derived Peptide 3.3
KFSSLYISQFIIMYS

SEQ ID NO: 328 - protein, 15 aa, Artificial Sequence
  FVIII Derived Peptide 3.4
FSSLYISQFIIMYSL

SEQ ID NO: 329 - protein, 15 aa, Artificial Sequence
  FVIII Derived Peptide 3.5
SSLYISQFIIMYSLD

SEQ ID NO: 330 - protein, 15 aa, Artificial Sequence
  FVIII Derived Peptide 3.6
SLYISQFIIMYSLDG

SEQ ID NO: 331 - protein, 15 aa, Artificial Sequence
  FVIII Derived Peptide 3.7
LYISQFIIMYSLDGK

SEQ ID NO: 332 - protein, 15 aa, Artificial Sequence
  FVIII Derived Peptide 3.8
YISQFIIMYSLDGKK

SEQ ID NO: 333 - protein, 15 aa, Artificial Sequence
  FVIII Derived Peptide 3.9
ISQFIIMYSLDGKKW

SEQ ID NO: 334 - protein, 15 aa, Artificial Sequence
  FVIII Derived Peptide 3.10
SQFIIMYSLDGKKWQ

SEQ ID NO: 335 - protein, 15 aa, Artificial Sequence
  FVIII Derived Peptide 3.11
QFIIMYSLDGKKWQT

SEQ ID NO: 336 - protein, 15 aa, Artificial Sequence
  FVIII Derived Peptide 4
PPIIARYIRLHPTHY

SEQ ID NO: 337 - protein, 15 aa, Artificial Sequence
  FVIII Derived Peptide 4.1
HNIFNPPIIARYIRL

SEQ ID NO: 338 - protein, 15 aa, Artificial Sequence
  FVIII Derived Peptide 4.2
NIFNPPIIARYIRLH

SEQ ID NO: 339 - protein, 15 aa, Artificial Sequence
  FVIII Derived Peptide 4.3
IFNPPIIARYIRLHP

SEQ ID NO: 340 - protein, 15 aa, Artificial Sequence
  FVIII Derived Peptide 4.4
FNPPIIARYIRLHPT

SEQ ID NO: 341 - protein, 15 aa, Artificial Sequence
  FVIII Derived Peptide 4.5
NPPIIARYIRLHPTH

SEQ ID NO: 342 - protein, 15 aa, Artificial Sequence
  FVIII Derived Peptide 4.6
PPIIARYIRLHPTHY

SEQ ID NO: 343 - protein, 14 aa, Artificial Sequence
  FVIII Derived Peptide 4.7
PEARYIRLHPTHYS

SEQ ID NO: 344 - protein, 15 aa, Artificial Sequence
  FVIII Derived Peptide 4.8
IIARYIRLHPTHYSI

SEQ ID NO: 345 - protein, 15 aa, Artificial Sequence
  FVIII Derived Peptide 4.9
IARYIRLHPTHYSIR

SEQ ID NO: 346 - protein, 15 aa, Artificial Sequence
  FVIII Derived Peptide 4.10
ARYERLHPTHYSIRS

SEQ ID NO: 347 - protein, 15 aa, Artificial Sequence
  FVIII Derived Peptide 4.11
RYIRLHPTHYSIRST

SEQ ID NO: 348 - protein, 16 aa, Artificial Sequence
  FVIII Derived Peptide 5
PPLLTRYLRIHPQSSW

SEQ ID NO: 349 - protein, 15 aa, Artificial Sequence
  FVIII Derived Peptide 6
MHTVNGYVNRSLPGL

SEQ ID NO: 350 - protein, 15 aa, Artificial Sequence
  FVIII Derived Peptide 7
LGQFLLFCHISSHQH

SEQ ID NO: 351 - protein, 15 aa, Artificial Sequence
  FVIII Derived Peptide 8
DTLLIIFKNQASRPY

SEQ ID NO: 352 - protein, 15 aa, Artificial Sequence
  FVIII Derived Peptide 8.1
YGEVGDTLLIIFKNQ

SEQ ID NO: 353 - protein, 15 aa, Artificial Sequence
  FVIII Derived Peptide 8.2
GEVGDTLLIIFKNQA

SEQ ID NO: 354 - protein, 15 aa, Artificial Sequence
  FVIII Derived Peptide 8.3
EVGDTLLIIFKNQAS

SEQ ID NO: 355 - protein, 15 aa, Artificial Sequence
  FVIII Derived Peptide 8.4
VGDTLLIIFKNQASR

SEQ ID NO: 356 - protein, 15 aa, Artificial Sequence
  FVIII Derived Peptide 8.5
GDTLLIIFKNQASRP

SEQ ID NO: 357 - protein, 15 aa, Artificial Sequence
  FVIII Derived Peptide 8.6
DTLLIIFKNQASRPY

SEQ ID NO: 358 - protein, 15 aa, Artificial Sequence
  FVIII Derived Peptide 8.7
TLLIIFKNQASRPYN

SEQ ID NO: 359 - protein, 15 aa, Artificial Sequence
  FVIII Derived Peptide 8.8
LLIIFKNQASRPYNI

SEQ ID NO: 360 - protein, 15 aa, Artificial Sequence
  FVIII Derived Peptide 8.9
LIIFKNQASRPYNIY

SEQ ID NO: 361 - protein, 15 aa, Artificial Sequence
  FVIII Derived Peptide 8.10
IIFKNQASRPYNIYP

SEQ ID NO: 362 - protein, 15 aa, Artificial Sequence
  FVIII Derived Peptide 8.11
IFKNQASRPYNGYPH

SEQ ID NO: 363 - protein, 15 aa, Artificial Sequence
  FVIII Derived Peptide 9
PRCLTRYYSSFVNME

SEQ ID NO: 364 - protein, 15 aa, Artificial Sequence
  FVIII Derived Peptide 9.1
PTKSDPRCLTRYYSS

SEQ ID NO: 365 - protein, 15 aa, Artificial Sequence
  FVIII Derived Peptide 9.2
TKSDPRCLTRYYSSF

SEQ ID NO: 366 - protein, 15 aa, Artificial Sequence
  FVIII Derived Peptide 9.3
KSDPRCLTRYYSSFV

SEQ ID NO: 367 - protein, 15 aa, Artificial Sequence
  FVIII Derived Peptide 9.4
SDPRCLTRYYSSFVN

SEQ ID NO: 368 - protein, 15 aa, Artificial Sequence
  FVIII Derived Peptide 9.5
DPRCLTRYYSSFVNM

SEQ ID NO: 369 - protein, 15 aa, Artificial Sequence
  FVIII Derived Peptide 9.6
PRCLTRYYSSFVNME

SEQ ID NO: 370 - protein, 15 aa, Artificial Sequence
  FVIII Derived Peptide 9.7
RCLTRYYSSFVNMER

SEQ ID NO: 371 - protein, 15 aa, Artificial Sequence
  FVIII Derived Peptide 9.8
CLTRYYSSFVNMERD

SEQ ID NO: 372 - protein, 15 aa, Artificial Sequence
  FVIII Derived Peptide 9.9
LTRYYSSFVNMERDL

SEQ ID NO: 373 - protein, 15 aa, Artificial Sequence
  FVIII Derived Peptide 9.10
TRYYSSFVNMERDLA

SEQ ID NO: 374 - protein, 15 aa, Artificial Sequence
  FVIII Derived Peptide 9.11
RYYSSFVNMERDLAS

SEQ ID NO: 375 - protein, 15 aa, Artificial Sequence
  FVIII Derived Peptide 10
TENIQRFLPNPAGVQ

SEQ ID NO: 376 - protein, 15 aa, Artificial Sequence
  FVIII Derived Peptide 11
DNIMVTFRNQASRPY

SEQ ID NO: 377 - protein, 15 aa, Artificial Sequence
  FVIII Derived Peptide 11.1
RAEVEDNIMVTFRNQ

SEQ ID NO: 378 - protein, 15 aa, Artificial Sequence
  FVIII Derived Peptide 11.2
AEVEDNIMVTFRNQA

SEQ ID NO: 379 - protein, 15 aa, Artificial Sequence
  FVIII Derived Peptide 11.3
EVEDNIMVTFRNQAS

SEQ ID NO: 380 - protein, 15 aa, Artificial Sequence
  FVIII Derived Peptide 11.4
VEDNIMVTFRNQASR

SEQ ID NO: 381 - protein, 15 aa, Artificial Sequence
  FVIII Derived Peptide 11.5
EDNTMVTFRNQASRP

SEQ ID NO: 382 - protein, 15 aa, Artificial Sequence
  FVIII Derived Peptide 11.6
DNIMVTFRNQASRPY

SEQ ID NO: 383 - protein, 15 aa, Artificial Sequence
  FVIII Derived Peptide 11.7
NIMVTFRNQASRPYS

SEQ ID NO: 384 - protein, 15 aa, Artificial Sequence
  FVIII Derived Peptide 11.8
IMVTFRNQASRPYSF

SEQ ID NO: 385 - protein, 15 aa, Artificial Sequence
  FVIII Derived Peptide 11.9
MVTFRNQASRPYSFY

SEQ ID NO: 386 - protein, 15 aa, Artificial Sequence
  FVIII Derived Peptide 11.10
VTFRNQASRPYSFYS

SEQ ID NO: 387 - protein, 15 aa, Artificial Sequence
  FVIII Derived Peptide 11.11
TFRNQASRPYSFYSS

SEQ ID NO: 388 - protein, 15 aa, Artificial Sequence
  FVIII Derived Peptide 12
RYLRIHPQSWVHQIA

SEQ ID NO: 389 - protein, 15 aa, Artificial Sequence
  FVIII Derived Peptide 12.1
PPLLTRYLRIHPQSW

SEQ ID NO: 390 - protein, 15 aa, Artificial Sequence
  FVIII Derived Peptide 12.2
PLLTRYLRIHPQSWV

SEQ ID NO: 391 - protein, 15 aa, Artificial Sequence
  FVIII Derived Peptide 12.3
LLTRYLRIHPQSWVH

SEQ ID NO: 392 - protein, 15 aa, Artificial Sequence
  FVIII Derived Peptide 12.4
LTRYLREHPQSWVHQ

SEQ ID NO: 393 - protein, 16 aa, Artificial Sequence
  FVIII Derived Peptide 12.5
TRYLRFFLPQSWVHQI

SEQ ID NO: 394 - protein, 15 aa, Artificial Sequence
  FVIII Derived Peptide 12.6
RYLRIHPQSWVHQIA

SEQ ID NO: 395 - protein, 15 aa, Artificial Sequence
  FVIII Derived Peptide 12.7
YLRIHPQSWVHQIAL

SEQ ID NO: 396 - protein, 15 aa, Artificial Sequence
  FVIII Derived Peptide 12.8
LRIHPQSWVHQIALR

SEQ ID NO: 397 - protein, 15 aa, Artificial Sequence
  FVIII Derived Peptide 12.9
RIHPQSWVHQIALRM

SEQ ID NO: 398 - protein, 15 aa, Artificial Sequence
  FVIII Derived Peptide 12.10
IHPQSWVHQIALRME

SEQ ID NO: 399 - protein, 15 aa, Artificial Sequence
  FVIII Derived Peptide 12.11
HPQSWVHQIALRMEV

SEQ ID NO: 400 - protein, 16 aa, Artificial Sequence
  FVIII Derived Peptide 1
LYISQFIIMMYSLDGK

SEQ ID NO: 401 - protein, 15 aa, Artificial Sequence
  FVIII Derived Peptide 2
FIIMYSLDGKKWQTY

SEQ ID NO: 402 - protein, 15 aa, Artificial Sequence
  FVIII Derived Peptide 3
PIIARYIRLHPTHYS

SEQ ID NO: 403 - protein, 15 aa, Artificial Sequence
  FVIII Derived Peptide 4
LIIFKNQASRPYNIY

SEQ ID NO: 404 - protein, 15 aa, Artificial Sequence
  FVIII Derived Peptide 5
LLIIFKNQASRPYNI

SEQ ID NO: 405 - protein, 15 aa, Artificial Sequence
  FVIII Derived Peptide 6
TLLIIFKNQASRPYN

SEQ ID NO: 406 - protein, 16 aa, Artificial Sequence
  FVIII Derived Peptide 7
GDTLLLIIFKNQASRP

SEQ ID NO: 407 - protein, 16 aa, Artificial Sequence
  FVIII Derived Peptide 8
VGDTLLLIIFKNQASR

SEQ ID NO: 408 - protein, 16 aa, Artificial Sequence
  FVIII Derived Peptide 9
EVGDTLLLIIFKNQAS

SEQ ID NO: 409 - protein, 15 aa, Artificial Sequence
  FVIII Derived Peptide 10
CLTRYYSSFVNMERD

SEQ ID NO: 410 - protein, 15 aa, Artificial Sequence
  FVIII Derived Peptide 11
EDNIMVTFRNQASRP

SEQ ID NO: 411 - protein, 15 aa, Artificial Sequence
  FVIII Derived Peptide 12
TRYLRIHPQSWVHQI

SEQ ID NO: 412 - protein, 15 aa, Artificial Sequence
  FVIII Derived Peptide 13
LTRYLRIHPQSWVHQ

SEQ ID NO: 413 - protein, 11 aa, Artificial Sequence
  Autoantibody Binding Peptide Related To Multiple Sclerosis 1
ASSRGYEDLRF

SEQ ID NO: 414 - protein, 10 aa, Artificial Sequence
  Autoantibody Binding Peptide Related To Multiple Sclerosis 2
LDNSYHDNPV

SEQ ID NO: 415 - protein, 10 aa, Artificial Sequence
  Autoantibody Binding Peptide Related To Multiple Sclerosis 3
LRAPAGLGAA

SEQ ID NO: 416 - protein, 10 aa, Artificial Sequence
  Autoantibody Binding Peptide Related To Multiple Sclerosis 4
GARCINAEQP

SEQ ID NO: 417 - protein, 10 aa, Artificial Sequence
  Autoantibody Binding Peptide Related To Multiple Sclerosis 5
YSCLKLYSFA

SEQ ID NO: 418 - protein, 10 aa, Artificial Sequence
  Autoantibody Binding Peptide Related To Multiple Sclerosis 6
EHATQNQVSV

SEQ ID NO: 419 - protein, 10 aa, Artificial Sequence
  Autoantibody Binding Peptide Related To Multiple Sclerosis 7
GTGSGQGEEA

SEQ ID NO: 420 - protein, 10 aa, Artificial Sequence
  Autoantibody Binding Peptide Related To Multiple Sclerosis 8
ADDNFSIPEG

SEQ ID NO: 421 - protein, 10 aa, Artificial Sequence
  Autoantibody Binding Peptide Related To Multiple Sclerosis 9
ASSRGYEDLR

SEQ ID NO: 422 - protein, 23 aa, Artificial Sequence
  Autoantibody Binding Peptide Related To Multiple Sclerosis 10
LDNSYHDNPVVSKELRIEGNQLT

SEQ ID NO: 423 - protein, 14 aa, Artificial Sequence
  Autoantibody Binding Peptide Related To Multiple Sclerosis 11
GARCINAEQPCQSP

SEQ ID NO: 424 - protein, 11 aa, Artificial Sequence
  Autoantibody Binding Peptide Related To Multiple Sclerosis 12
YSCLKLYSFAN

SEQ ID NO: 425 - protein, 15 aa, Artificial Sequence
  MBP 134-148
YKSAHKGFKGVDAQG

SEQ ID NO: 426 - protein, 15 aa, Artificial Sequence
  MBP 135-149
KSAHKGFKGVDAQGT

SEQ ID NO: 427 - protein, 15 aa, Artificial Sequence
  MBP 136-150
SAHKGFKGVDAQGTL

SEQ ID NO: 428 - protein, 15 aa, Artificial Sequence
  MBP 137-151
AHKGFKGVDAQGTLS

SEQ ID NO: 429 - protein, 15 aa, Artificial Sequence
  MBP 138-152
HKGFKGVDAQGTLSK

SEQ ID NO: 430 - protein, 15 aa, Artificial Sequence
  MBP 139-153
KGFKGVDAQGTLSKI

SEQ ID NO: 431 - protein, 15 aa, Artificial Sequence
  MBP 140-154
GFKGVDAQGTLSKIF

SEQ ID NO: 432 - protein, 17 aa, Artificial Sequence
  MBP 83-99
ENPVVHFFKNIVTPRTP

SEQ ID NO: 433 - protein, 15 aa, Artificial Sequence
  MBP 80-94
TQDENPVVHFFKNIV

SEQ ID NO: 434 - protein, 15 aa, Artificial Sequence
  MBP 81-95
QDENPVVHFFKNIVT

SEQ ID NO: 435 - protein, 15 aa, Artificial Sequence
  MBP 82-96
DENPVVHFFKNIVTP

SEQ ID NO: 436 - protein, 15 aa, Artificial Sequence
  MBP 83-97
ENPVVHFFKNIVTPR

SEQ ID NO: 437 - protein, 15 aa, Artificial Sequence
  MBP 84-98
MPVVHFFKNIVTPRT

SEQ ID NO: 438 - protein, 15 aa, Artificial Sequence
  MBP 130-144
RASDYKSAHKGFKGV

SEQ ID NO: 439 - protein, 15 aa, Artificial Sequence
  MBP 131-145
ASDYKSAHKGFKGVD

SEQ ID NO: 440 - protein, 15 aa, Artificial Sequence
  MBP 132-146
SDYKSAHKGFKGVDA

SEQ ID NO: 441 - protein, 15 aa, Artificial Sequence
  MBP 133-147
DYKSAHKGFKGVDAQ

SEQ ID NO: 442 - protein, 247 aa, Homo sapiens; identical to SEQ ID NO: 6

SEQ ID NO: 443 - protein, 208 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFGQFLEELRNPF

SEQ ID NO: 444 - protein, 224 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFESFGVLGPQVKEPKKTGQFLEELRNPF

SEQ ID NO: 445 - protein, 131 aa, Homo sapiens
MASLSRPSLPSCLCSFLLLLLLQVSSSYADPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 446 - protein, 252 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELLFHLEALSG

SEQ ID NO: 447 - protein, 213 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFGQFLEELLFHLEALSG

SEQ ID NO: 448 - protein, 229 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFESFGVLGPQVKEPKKTGQFLEELLFHLEALSG

SEQ ID NO: 449 - protein, 206 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFLFHLEALSG

SEQ ID NO: 450 - protein, 203 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFGKFRHV

SEQ ID NO: 451 - protein, 171 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEVSHSVTQDWLQWHDHGSLQPPPPRLK

SEQ ID NO: 452 - protein, 206 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFVFHLEALSG

SEQ ID NO: 453 - protein, 108 aa, Homo sapiens
MASLSRPSLPSCLCSFLLLLLLQVSSSYADPFYWVSPGVLVLLAVLPVLLLQITVGLIFLCLQYRLRGKLRAEIENLHRTFESFGVLGPQVKEPKKTGQFLEELRNPF

SEQ ID NO: 454 - protein, 295 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRKFSSLCYKQRIKSQERETEATRGRGGLLRDHIPRGKEELESLGGGKTPPGR

SEQ ID NO: 455 - protein, 90 aa, Homo sapiens
SLSRPSLPSCLCSFLLLLLLQVSSSYADPFYWVSPGVLVLLAVLPVLLLQITVGLVFLCLQYRLRGKLRAEIENLHRTFGQFLEELRNPF

SEQ ID NO: 456 - protein, 106 aa, Homo sapiens
SLSRPSLPSCLCSFLLLLLLQVSSSYADPFYWVSPGVLVLLAVLPVLLLQITVGLVFLCLQYRLRGKLRAEIENLHRTFESFGVLGPQVKEPKKTGQFLEELRNPF

SEQ ID NO: 457 - protein, 171 aa, Homo sapiens
SLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEGLVAVARSWLIAASTSQAQVILLTQPLE

SEQ ID NO: 458 - protein, 53 aa, Homo sapiens
SLSRPSLPSCLCSFLLLLLLQVSSSYAVSHSVTQDWLQWHDHGSLQPPPPRLK

SEQ ID NO: 459 - protein, 55 aa, Homo sapiens
SLSRPSLPSCLCSFLLLLLLQVSSSYAGLVAVARSWLIAASTSQAQVILLTQPLE

SEQ ID NO: 460 - protein, 151 aa, Homo sapiens
SLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEAPLKQRSR

SEQ ID NO: 461 - DNA, 744 bp, Mus musculus
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctctgtaggccttgtattcctcttcctgcagcacagactgagaggaaaacttcgtgcagaagtagagaatctccatcggacttttgatcctcacttcctgagggtgccctgctggaagataacactgtttgttattgtgcctgttcttggacccctggttgccttgatcctgtgctacaactggctgcaccgaagactggcaggacagtttcttgaagagctaagaaaccccttttga

SEQ ID NO: 462 - DNA, 441 bp, Artificial Sequence
  human chymotrypsinogen B2 signal peptide+ Mus musculus mini-MOG cDNA (start to stop)
atggcctttctgtggctgctgtcctgctgggccctgctggggaccacctttggcggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgacttga

SEQ ID NO: 463 - protein, 150 aa, Artificial Sequence
  TM1-miniMOG (aa29-178)
GQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVPVGLVFLFLT

SEQ ID NO: 464 - protein, 141 aa, Artificial Sequence
  TM0.6-miniMOG
GQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVP

SEQ ID NO: 465 - protein, 134 aa, Artificial Sequence
  TM0.3-miniMOG
GQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVP

SEQ ID NO: 466 - protein, 117 aa, Artificial Sequence
  Mus musculus MOG 1-117
GQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVED

SEQ ID NO: 467 - protein, 117 aa, Artificial Sequence
  Homo sapiens MOG 1-117
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVED

SEQ ID NO: 468 - protein, 117 aa, Artificial Sequence
  Macaca mulatta MOG 1-117
GQFRVIGPRQPIRALVGDEVELPCRISPGKNATGMEVGWYRPPFSRVVHLYRNGRDQDGEQAPEYRGRTELLKDAIGEGKVTLRIRNVRFSDEGGFTCFFRDHSYQEEAAIELKVED

SEQ ID NO: 469 - protein, 117 aa, Artificial Sequence
  Callithrix jacchus MOG 1-117
GQFRVIGPSHPIQALVGDAAELPCRISPGKNATGMEVGWYRSPFSRVVHLYRNGKDQDGEQAPEYRGRTELLKDDIGEGKVTLKIRNVRFPDEGGFTCFFRDHSYQEEAAMQLKVED

SEQ ID NO: 470 - protein, 135 aa, Artificial Sequence
  human chymotrypsinogen B2 signal peptide+ Mus musculus MOG 1-117
MAFLWLLSCWALLGTTFGGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVED

SEQ ID NO: 471 - protein, 135 aa, Artificial Sequence
  human chymotrypsinogen B2 signal peptide+ Homo sapiens MOG 1-117
MAFLWLLSCWALLGTTFGGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVED

SEQ ID NO: 472 - protein, 135 aa, Artificial Sequence
  human chymotrypsinogen B2 signal peptide+ Macaca mulatta MOG 1-117
MAFLWLLSCWALLGTTFGGQFRVIGPRQPIRALVGDEVELPCRISPGKNATGMEVGWYRPPFSRVVHLYRNGRDQDGEQAPEYRGRTELLKDAIGEGKVTLRIRNVRFSDEGGFTCFFRDHSYQEEAAIELKVED

SEQ ID NO: 473 - protein, 135 aa, Artificial Sequence
  human chymotrypsinogen B2 signal peptide+ Callithrix jacchus MOG 1-117
MAFLWLLSCWALLGTTFGGQFRVIGPSHPIQALVGDAAELPCRISPGKNATGMEVGWYRSPFSRVVHLYRNGKDQDGEQAPEYRGRTELLKDDIGEGKVTLKIRNVRFPDEGGFTCFFRDHSYQEEAAMQLKVED

SEQ ID NO: 474 - DNA, 447 bp, Artificial Sequence
  TM1-miniMOG (aa29-178)
ggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctctgtaggccttgtattcctcttcctg

SEQ ID NO: 475 - DNA, 423 bp, Artificial Sequence
  TM0.6-miniMOG (aa29-169)
ggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctct

SEQ ID NO: 476 - DNA, 402 bp, Artificial Sequence
  TM0.3-miniMOG (aa29-162)
ggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcct

SEQ ID NO: 477 - protein, 27 aa, Artificial Sequence
  PLP1 (aa37-63)
HEALTGTEKLIETYFSKNYQDYEYLIN

SEQ ID NO: 478 - protein, 54 aa, Artificial Sequence
  PLP1 + TM1 (aa10-63)
CLVGAPFASLVATGLCFFGVALFCGCGHEALTGTEKLIETYFSKNYQDYEYLIN

SEQ ID NO: 479 - protein, 63 aa, Artificial Sequence
  PLP2 (aa89-151)
EGFYTTGAVRQIFGDYKTTICGKGLSATVTGGQKGRGSRGQHQAHSLERVCHCLGKWLGHPDK

SEQ ID NO: 480 - protein, 88 aa, Artificial Sequence
  PLP2 + TM2 (aa64-151)
VIHAFQYVIYGTASFFFLYGALLLAEGFYTTGAVRQIFGDYKTTICGKGLSATVTGGQKGRGSRGQHQAHSLERVCHCLGKWLGHPDK

SEQ ID NO: 481 - protein, 56 aa, Artificial Sequence
  PLP3 (aa178-233)
FNTWTTCQSIAFPSKTSASIGSLCADARMYGVLPWNAFPGKVCGSNLLSICKTAEF

SEQ ID NO: 482 - protein, 82 aa, Artificial Sequence
  PLP3 + TM3 (aa152-233)
FVGITYALTVVWLLVFACSAVPVYIYFNTWTTCQSIAFPSKTSASIGSLCADARMYGVLPWNAFPGKVCGSNLLSICKTAEF

SEQ ID NO: 483 - protein, 17 aa, Artificial Sequence
  PLP4 (aa261-277)
ATYNFAVLKLMGRGTKF

SEQ ID NO: 484 - protein, 44 aa, Artificial Sequence
  PLP4 + TM4 (aa234-277)
QMTFHLFIAAFVGAAATLVSLLTFMIAATYNFAVLKLMGRGTKF

SEQ ID NO: 485 - DNA, 81 bp, Artificial Sequence
  PLP1
cacgaggccctgaccggcaccgagaagctgatcgagacctacttcagcaagaactaccaggactacgagtacctgatcaac

SEQ ID NO: 486 - DNA, 162 bp, Artificial Sequence
  PLP1 + TM1
tgcctggtgggcgcccccttcgccagcctggtggccaccggcctgtgcttcttcggcgtggccctgttctgcggctgcggccacgaggccctgaccggcaccgagaagctgatcgagacctacttcagcaagaactaccaggactacgagtacctgatcaac

SEQ ID NO: 487 - DNA, 189 bp, Artificial Sequence
  PLP2
gagggcttctacaccaccggcgccgtgaggcagatcttcggcgactacaagaccaccatctgcggcaagggcctgagcgccaccgtgaccggcggccagaagggcaggggcagcaggggccagcaccaggcccacagcctggagagggtgtgccactgcctgggcaagtggctgggccaccccgacaag

SEQ ID NO: 488 - DNA, 264 bp, Artificial Sequence
  PLP2 + TM2
gtgatccacgccttccagtacgtgatctacggcaccgccagcttcttcttcctgtacggcgccctgctgctggccgagggcttctacaccaccggcgccgtgaggcagatcttcggcgactacaagaccaccatctgcggcaagggcctgagcgccaccgtgaccggcggccagaagggcaggggcagcaggggccagcaccaggcccacagcctggagagggtgtgccactgcctgggcaagtggctgggccaccccgacaag

SEQ ID NO: 489 - DNA, 168 bp, Artificial Sequence
  PLP3
ttcaacacctggaccacctgccagagcatcgccttccccagcaagaccagcgccagcatcggcagcctgtgcgccgacgccaggatgtacggcgtgctgccctggaacgccttccccggcaaggtgtgcggcagcaacctgctgagcatctgcaagaccgccgagttc

SEQ ID NO: 490 - DNA, 240 bp, Artificial Sequence
  PLP3 + TM3
ggcatcacctacgccctgaccgtggtgtggctgctggtgttcgcctgcagcgccgtgcccgtgtacatctacttcaacacctggaccacctgccagagcatcgccttccccagcaagaccagcgccagcatcggcagcctgtgcgccgacgccaggatgtacggcgtgctgccctggaacgccttccccggcaaggtgtgcggcagcaacctgctgagcatctgcaagaccgccgagttc

SEQ ID NO: 491 - DNA, 51 bp, Artificial Sequence
  PLP4
gccacctacaacttcgccgtgctgaagctgatgggcaggggcaccaagttc

SEQ ID NO: 492 - DNA, 132 bp, Artificial Sequence
  PLP4 + TM4
cagatgaccttccacctgttcatcgccgccttcgtgggcgccgccgccaccctggtgagcctgctgaccttcatgatcgccgccacctacaacttcgccgtgctgaagctgatgggcaggggcaccaagttc